(19) United States
(12) Patent Application Publication
(10) Pub. No.: US 2025/0354213 A1
(43) Pub. Date: Nov. 20, 2025

(54) METHODS RELATING TO RESPIRATORY HEALTH THERAPEUTICS

(71) Applicants: Northwestern University, Evanston, IL (US); Vanderbilt University, Nashville, TN (US); Brigham and Women's Hospital, Somerville, MA (US)

(72) Inventors: Gabrielle Yi-Hui LIU, Evanston, IL (US); Ravi KALHAN, Evanston, IL (US); George R. WASHKO, Somerville, MA (US); Bina CHOI, Somerville, MA (US); Ravi SHAH, Nashville, TN (US); Andrew PERRY, Nashville, TN (US)

(21) Appl. No.: 19/170,650

(22) Filed: Apr. 4, 2025

Related U.S. Application Data
(60) Provisional application No. 63/574,697, filed on Apr. 4, 2024.

(51) Int. Cl. C12Q 1/6883; G01N 33/68

(52) U.S. Cl. CPC C12Q 1/6883; G01N 33/6893; C12Q 2600/158; G01N 2800/122

(57) ABSTRACT

The present disclosure provides include kits, compositions, and methods related to impaired respiratory health (e.g., diseases and conditions relating to lung injury). In particular, the present disclosure provides include kits, compositions, and methods for quantifying biomarkers (e.g., proteins, nucleic acids, etc.) associated with an accelerated decline in rapid forced expiratory volume in 1 s (FEV1) and assessing the risk of, monitoring, treating and/or preventing respiratory diseases and conditions.

### CROSS REFERENCE TO RELATED APPLICATIONS

This application claims the benefit of U.S. Provisional Patent Application No. 63/574,697, filed Apr. 4, 2025, which is incorporated by reference herein in its entirety.

### GOVERNMENT SUPPORT

This invention was made with government support under HL162318 and HL122477 awarded by the National Institutes of Health. The government has certain rights in the invention.

### FIELD

The present disclosure provides include kits, compositions, and methods related to impaired respiratory health (e.g., diseases and conditions relating to lung injury). In particular, the present disclosure provides include kits, compositions, and methods for quantifying biomarkers (e.g., proteins, nucleic acids, etc.) associated with an accelerated decline in rapid forced expiratory volume in 1 s (FEV1) and assessing the risk of, monitoring, treating and/or preventing respiratory diseases and conditions.

### BACKGROUND

Understanding the complex biochemical processes that regulate lung and immune system development which, subsequently, affects respiratory health is important to the primary prevention of lung diseases. Prior studies of lung function have been limited to short intervals (1-7 year) FEV1 declines and lacked clear implications on long-term respiratory health, especially at an early modifiable period in disease. Kits, compositions, and methods are needed to identify biomarkers associated with respiratory health.

### SUMMARY

The present disclosure provides include kits, compositions, and methods related to impaired respiratory health (e.g., diseases and conditions relating to lung injury). In particular, the present disclosure provides include kits, compositions, and methods for quantifying biomarkers (e.g., proteins, nucleic acids, etc.) associated with an accelerated decline in rapid forced expiratory volume in 1 s (FEV1) and assessing the risk of, monitoring, treating and/or preventing respiratory diseases and conditions.

Embodiments of the present disclosure includes methods comprising quantitating, within a biological sample from a subject, the levels of two or more biomarkers from a first panel of protein biomarkers comprising: SLAM family member 7; Interleukin-36 alpha; Oxytocin-neurophysin 1; Fatty acid-binding protein, adipocyte; Axin-2; Interleukin-17 receptor E; Orphan sodium- and chloride-dependent neurotransmitter transporter NTT5; BPI fold-containing family B member 1; Galectin-3-binding protein; Nuclear distribution protein nudE-like 1; C-C motif chemokine 22; Liver-expressed antimicrobial peptide 2; Tissue-type plasminogen activator; Protein S100-A9; Marginal zone B- and B1-cell-specific protein; C-C motif chemokine 18; Piezo-type mechanosensitive ion channel component 1; Glutathione S-transferase A1; Ribonuclease K6; Amiloride-sensitive amine oxidase [copper-containing]; Gastrokine-2; Protocadherin gamma-C3; Macrophage scavenger receptor types I and II: Extracellular domain; Lipopolysaccharide-binding protein; C-C motif chemokine 3; Peroxidasin homolog; Scavenger receptor class B member 1; Ephrin type-B receptor 3; SLAM family member 8; Endothelial cell-derived lipase; Thrombospondin-2; Glycerol-3-phosphate dehydrogenase [NAD (+)], cytoplasmic; Estradiol 17-beta-dehydrogenase 1; Trafficking protein particle complex subunit 3; Serine protease HTRA1; Complement C3d fragment; Macrophage-capping protein; DnaJ homolog subfamily B member 9; Triggering receptor expressed on myeloid cells 2; Angiopoietin-2; Complement C3b, inactivated; Retinoic acid receptor responder protein 2; Complement component C9; Malignant T-cell-amplified sequence 1; Leukocyte immunoglobulin-like receptor subfamily A member 5; Glycerol-3-phosphate dehydrogenase [NAD (+)], cytoplasmic; Histone H2A type 1-A; Choline/ethanolamine kinase; Histone H2B type 1-K; Insulin; Myeloblastin; Growth hormone receptor; Serine/arginine-rich splicing factor 7; Interleukin-1 receptor antagonist protein; T-cell immunoglobulin and mucin domain-containing protein 4; Vesicular integral-membrane protein VIP36; V-set and transmembrane domain-containing protein 2-like protein; Transformer-2 protein homolog beta; E-selectin; Cytosolic Fe-S cluster assembly factor NUBP2; Ficolin-1; Gamma-glutamyl hydrolase; Leukocyte cell-derived chemotaxin-2; Inhibin beta A chain; Collagen alpha-3 (VI) chain: Bovine pancreatic trypsin inhibitor/Kunitz inhibitor domain, isoform 1; Marginal zone B- and B1-cell-specific protein; Regulator of G-protein signaling 4; C-C motif chemokine 21; Angiopoietin-2; Retinoblastoma-like protein 2; 5-hydroxytryptamine receptor 6; Protein S100-A12; Inhibin beta C chain; Bone sialoprotein 2; V-set and immunoglobulin domain-containing protein 4; Intercellular adhesion molecule 1; Triggering receptor expressed on myeloid cells 1; Collagen alpha-1 (XXVIII) chain; Olfactomedin-like protein 3; Tumor necrosis factor receptor superfamily member 10A; Serine/threonine-protein phosphatase 1 regulatory subunit 10; IGF-like family receptor 1; Thyroid transcription factor 1-associated protein 26; Tyrosine-protein phosphatase non-receptor type 7; G antigen 2; Replication initiator 1; ADAMTS-like protein 2; WAP four-disulfide core domain protein 2; Protein phosphatase 1 regulatory subunit 1A; PIH1 domain-containing protein 2; Rho GTPase-activating protein 36; 2-methoxy-6-polyprenyl-1,4-benzoquinol methylase, mitochondrial; Colipase-like protein 1; Sialic acid-binding Ig-like lectin 12: Ig-like C2-type 2 domain, Isoform short; Osteopetrosis-associated transmembrane protein 1; Cystatin B; Delta-like protein 1; Tumor necrosis factor receptor superfamily member 6; Insulin-like growth factor-binding protein 4; Lysozyme C; Protein FAM19A5; FXYD domain-containing ion transport regulator 6; Alpha-1-antichymotrypsin complex; Trafficking protein particle complex subunit 5; Protein FAM234B: N-term; Heat shock 70 kDa protein 1B; Cellular retinoic acid-binding protein 2; Z-DNA-binding protein 1; Transcriptional repressor protein YY1; Beta-2-microglobulin; Triggering receptor expressed on myeloid cells 2; Far upstream element-binding protein 2; Fc receptor-like protein 3; ATPase family AAA domain-containing protein 2; Galectin-9; Calcipressin-3; Plastin-2; Leucine-rich repeats and immunoglobulin-like domains protein 1; Complement factor B; Heat shock 70 kDa protein 1A; E3 ubiquitin-protein ligase RAD18; Syndecan-3; Antizyme inhibitor 1; Complement component C9; Ubiquitin-conjugating enzyme E2 G2; Interleukin-2; Holo-Transcobalamin-2; Ephrin-A1; Oncoprotein-induced transcript 3 protein; Stathmin-3; Tumor necrosis factor receptor superfamily member 1B; Asialoglycoprotein receptor 1; Methionine-R-sulfoxide reductase B1; CREB-binding protein; Serine/arginine-rich splicing factor 6; Synembryn-A; Myc target protein 1; Heparin cofactor 2; Ataxin-2-binding protein 1; GTPase IMAP family member 6; Galectin-4; Leukocyte immunoglobulin-like receptor subfamily A member 5; Thymidine kinase, cytosolic; ADP-ribosylation factor-like protein 15; DCN1-like protein 1; Coagulation factor IXab; Coagulation factor IX; Heat shock 70 kDa protein 1A; Tumor necrosis factor receptor superfamily member 1B; Sperm surface protein Sp17; Complement Clr subcomponent-like protein; Peptidyl-prolyl cis-trans isomerase C; Protein FAM204A; Interleukin-17C; Gamma-aminobutyric acid receptor-associated protein-like 1; Legumain; Inactive ribonuclease-like protein 10; Vitronectin; HLA class II histocompatibility antigen, DR beta 3 chain; Heat shock 70 kDa protein 1A; Tolloid-like protein 1; Macrophage receptor MARCO; Four and a half LIM domains protein 1; Gamma-aminobutyric acid receptor-associated protein; Killer cell immunoglobulin-like receptor 2DS4; Protein BUD31 homolog; Alpha-aminoadipic semialdehyde dehydrogenase; Inactive serine protease 35; Ubiquitin-conjugating enzyme E2 D2; Complement factor D; Dipeptidyl peptidase 1; Heat shock cognate 71 kDa protein; Antileukoproteinase; WD repeat-containing protein 5; Out at first protein homolog; Neuroblastoma suppressor of tumorigenicity 1; Tumor necrosis factor ligand superfamily member 11; Kynureninase; Ephrin-A4; EGF-containing fibulin-like extracellular matrix protein 1; Tumor necrosis factor receptor superfamily member 19L; Serum amyloid P-component; Complement C2; Follistatin-related protein 3; Cystatin-C; Serine/threonine-protein kinase 17B; Protein RIC-3; Leukocyte immunoglobulin-like receptor subfamily B member 4; Metalloproteinase inhibitor 1; C-X-C motif chemokine 16; Fibulin-5; Transmembrane gamma-carboxyglutamic acid protein 1: Cytoplasmic domain; C4b-binding protein alpha chain; Tumor necrosis factor ligand superfamily member 15; Secreted and transmembrane protein 1; Heat shock 70 kDa protein 1A; Complement Clq tumor necrosis factor-related protein 5; Ganglioside GM2 activator; Gamma-aminobutyric acid receptor-associated protein; DnaJ homolog subfamily C member 11; Atrial natriuretic factor; D-dimer; RecQ-mediated genome instability protein 1; von Willebrand factor A domain-containing protein 1; Pituitary adenylate cyclase-activating polypeptide 38; Uncharacterized protein C14orf93; Gem-associated protein 6; Matrix Gla protein; Guanine nucleotide exchange factor VAV3; Inactive dipeptidyl peptidase 10; Plastin-2; Fibrinogen gamma chain; Paraspeckle component 1; Calcium-dependent phospholipase A2; Complement Clq tumor necrosis factor-related protein 1; Integrin a5b1; Calpain-9; Cadherin-1; Complement factor H-related protein 1; Complement factor H; Antithrombin-III; Apolipoprotein C-I; Neurogenic locus notch homolog protein 1; Heat shock 70 kDa protein 12A; Kallistatin; Glutathione peroxidase 3; Kallistatin; Prostasin; Repulsive guidance molecule A; Serum albumin; Epidermal growth factor receptor; Protein SCO1 homolog, mitochondrial; Mitogen-activated protein kinase 6; B melanoma antigen 3; Heparan-sulfate 6-O-sulfotransferase 3; Platelet endothelial aggregation receptor 1: Extracellular domain; Neural cell adhesion molecule L1-like protein; Neuropeptide S; Ephrin type-A receptor 4; Beta-hexosaminidase subunit beta; RGM domain family member B; Leucine-rich repeats and immunoglobulin-like domains protein 3; Melanoma-associated antigen MUC18; Zinc finger protein 230; Inter-alpha-trypsin inhibitor heavy chain H2; Tetratricopeptide repeat protein 33; Leukemia inhibitory factor receptor; Glycosaminoglycan xylosylkinase; Methylglutaconyl-CoA hydratase, mitochondrial; Endothelial cell-selective adhesion molecule; Neurotrimin; MAD2L1-binding protein; Growth/differentiation factor 2; Glucosidase 2 subunit beta; Desmoglein-2; Contactin-1; Neural cell adhesion molecule L1-like protein; Inter-alpha-trypsin inhibitor heavy chain H1; Slit homolog 2 protein; Heat shock 70 kDa protein 1A; Sodium/potassium-transporting ATPase subunit beta-2; Speriolin-like protein; Leucine-rich repeat transmembrane neuronal protein 2; Endothelial cell-selective adhesion molecule; Mediator of RNA polymerase II transcription subunit 11; Anosmin-1; Microtubule-associated proteins 1A/1B light chain 3 beta 2; Apolipoprotein A-IV; Beta-1,4-galactosyltransferase 2; Neurotrimin; Interleukin-1 receptor type 1; Apolipoprotein D; Dihydrolipoyl dehydrogenase, mitochondrial; Gliomedin; Serine protease 57; Interleukin-19; Inactive tyrosine-protein kinase transmembrane receptor ROR1; Carbonyl reductase [NADPH] 3; Kunitz-type protease inhibitor 2; Endothelial cell-specific molecule 1; Bone morphogenetic protein receptor type-1A; 15 kDa selenoprotein; Complement Clq-like protein 3; von Willebrand factor A domain-containing protein 2; Anthrax toxin receptor 2; Semaphorin-3G; Tyrosine-protein kinase SYK: Protein kinase domain; Normal mucosa of esophagus-specific gene 1 protein; Ciliary neurotrophic factor receptor subunit alpha; SLIT and NTRK-like protein 4; Protocadherin-9; Uronyl 2-sulfotransferase; N-acetylglucosamine-1-phosphodiester alpha-N-acetylglucosaminidase; Tubulointerstitial nephritis antigen-like; Cystatin-M; Dickkopf-related protein 3; Neural cell adhesion molecule 2; Polypeptide N-acetylgalactosaminyltransferase 4; Ephrin type-A receptor 4; Vesicular, overexpressed in cancer, prosurvival protein 1; Dual specificity protein phosphatase 13 isoform A; Ectonucleotide pyrophosphatase/phosphodiesterase family member 5; NT-3 growth factor receptor; Endothelial cell-selective adhesion molecule; Dipeptidase 1; 3-mercaptopyruvate sulfurtransferase; Trafficking protein particle complex subunit 6B; Cell adhesion molecule 2; Anthrax toxin receptor 1; Dickkopf-related protein 4; Gamma-glutamyltransferase 5; Seizure 6-like protein; EMILIN-3: region 1; Klotho; Protocadherin-10: Extracellular domain; Transmembrane protein 132A; Syntaxin-1A; WAP, Kazal, immunoglobulin, Kunitz and NTR domain-containing protein 2; Interleukin-2 receptor subunit beta; Acetylcholinesterase; Contactin-2; Coiled-coil domain-containing protein 126; Alpha-amylase 2B; Integrin alpha V beta 3; WAP, Kazal, immunoglobulin, Kunitz and NTR domain-containing protein 2; Interleukin-1 Receptor accessory protein; Glyoxylate reductase/hydroxypyruvate reductase; Neural cell adhesion molecule 1, 120 kDa isoform; Secretogranin-3; Neural cell adhesion molecule 1; Netrin receptor UNC5D; Alpha-amylase 2B; Alpha-1,3-mannosyltransferase ALG2; Matrix-remodeling-associated protein 8: Extracellular domain; Receptor-type tyrosine-protein phosphatase delta; Interleukin-1 Receptor accessory protein; A disintegrin and metalloproteinase with thrombospondin motifs 13; Dickkopf-related protein 3; Gamma-enolase; Glypican-3; Kynurenine-oxoglutarate transaminase 3; Calcium and integrin-binding protein 1; Prostaglandin F2 receptor negative regulator; SLIT and NTRK-like protein 5; Activating signal cointegrator 1 complex subunit 2; DNA-directed RNA polymerases I, II, and III subunit RPABC4; Brevican core protein; Peroxisomal carnitine O-octanoyltransferase; Neuronal pentraxin receptor; Ciliary neurotrophic factor receptor subunit alpha; Netrin receptor UNC5D; Biglycan; Adhesion G protein-coupled receptor F5; SLIT and NTRK-like protein 1; Serine-rich single-pass membrane protein 1; IgLON family member 5; Voltage-dependent calcium channel subunit alpha-2/delta-3; Pancreatic alpha-amylase; Ephrin type-A receptor 6; Interleukin-35; Integrin αIIb1; Adiponectin; Fc receptor-like protein 4: Extracellular domain; Histone-lysine N-methyltransferase EHMT2; THAP domain-containing protein 4; Interleukin-27 subunit beta; Myelin-associated glycoprotein; Pancreatic triacylglycerol lipase; Cerebellin-2; Cysteine-rich with EGF-like domain protein 1; Ecto-ADP-ribosyltransferase 3; Ecto-ADP-ribosyltransferase 3; AP-1 complex-associated regulatory protein; Neuronal pentraxin receptor; Neurocan core protein; Cerebellin-1; Cystatin-SA; ADP-ribosylation factor 4; DCC; Amyloid-like protein 1; Pyruvate dehydrogenase E1 component subunit alpha, testis-specific form, mitochondrial; Advanced glycosylation end product-specific receptor, soluble; Galactosylgalactosylxylosylprotein 3-beta-glucuronosyltransferase 1; Oligodendrocyte-myelin glycoprotein; Lactase-phlorizin hydrolase; Insulin-like growth factor-binding protein 1; SLIT and NTRK-like protein 3; Carbonic anhydrase 6; and Carbonic anhydrase 6.

Embodiments of the present disclosure includes methods comprising quantitating, within a biological sample from a subject, the levels of two or more biomarkers from a second panel of protein biomarkers comprising: Nuclear distribution protein nudE-like 1; Ribonuclease pancreatic; Estradiol 17-beta-dehydrogenase 1; Phospholipase A2, membrane associated; Protein S100-A9; Triggering receptor expressed on myeloid cells 1; Ribonuclease K6; Cystatin B; Osteopetrosis-associated transmembrane protein 1; Delta-like protein 1; Retinoic acid receptor responder protein 2; 5-hydroxytryptamine receptor 6; 2-methoxy-6-polyprenyl-1,4-benzoquinol methylase, mitochondrial; Collagen alpha-1 (XXVIII) chain; Collagen alpha-3 (VI) chain: Bovine pancreatic trypsin inhibitor/Kunitz inhibitor domain, isoform 1; Corticotropin-releasing factor-binding protein; Complement Clr subcomponent-like protein; Gamma-aminobutyric acid receptor-associated protein; Gamma-aminobutyric acid receptor-associated protein-like 1; Serine/threonine-protein kinase 17B; Leukemia inhibitory factor receptor; Hedgehog-interacting protein; Semaphorin-3G; Protein FAM177A1; Neural cell adhesion molecule 1; SLIT and NTRK-like protein 4; Tubulointerstitial nephritis antigen-like; NT-3 growth factor receptor; Uronyl 2-sulfotransferase; Protocadherin-9; Seizure 6-like protein; Neurogenic locus notch homolog protein 2; WAP, Kazal, immunoglobulin, Kunitz and NTR domain-containing protein 2; Dickkopf-related protein 3; IgLON family member 5; Histatin-3; WAP, Kazal, immunoglobulin, Kunitz and NTR domain-containing protein 2; Brevican core protein; Cerebellin-2; Myelin-associated glycoprotein; Alpha-amylase 2B; Neurocan core protein; Mannan-binding lectin serine protease 1: Mannan-binding lectin serine protease 1 heavy chain; Oligodendrocyte-myelin glycoprotein; Cystatin-D; Amyloid-like protein 1; Carbonic anhydrase 6; and Carbonic anhydrase 6.

In some embodiments, the two or more biomarkers are a protein or a cDNA.

In some embodiments, quantifying comprises a biophysical technique.

In some embodiments, the biophysical technique comprises using a point-of-care device.

In some embodiments, the point-of-care device uses binding agents and a detection/quantification methodology.

In some embodiments, the binding agents are antibodies, antibody fragments, or aptamers.

In some embodiments, the detection/quantification methodology is fluorescence, luminescence, or electrochemical detection.

In some embodiments, the biological sample is plasma or blood.

In some embodiments, the biological sample is obtained from a subject that suffers from impaired respiratory health.

In some embodiments, the biological sample is obtained from a subject that is at risk of impaired respiratory health.

In some embodiments, the biomarkers are associated with advanced parenchymal diseases.

In some embodiments, the advanced parenchymal diseases comprise lung injury, inflammation, pulmonary fibrosis, chronic obstructive pulmonary disease (COPD) and emphysema, and lung cancer.

In some embodiments, assessing the lung health of a subject comprises (a) quantifying levels of a panel of one or more biomarkers in a biological sample from the subject according to the method of one of claims 1-15; and (b) comparing the level of each one or more biomarker in the panel to a control or threshold value for each, wherein a significant difference between the level of one or more of the biomarkers from the control or threshold value is indicative of a impaired respiratory health.

In some embodiments, the panel comprises 50 or fewer biomarkers.

In some embodiments, the panel comprises 20 or fewer biomarkers.

In some embodiments, the control or threshold value is based on a population average for healthy subjects.

In some embodiments, comparing the level of each biomarker in the panel to a control or threshold value for each comprises calculating the difference between the level of each biomarker in the panel to a control or threshold value for each.

In some embodiments, comparing the level of each biomarkers in the panel to a control or threshold value for each further comprises dividing the difference by the standard deviation for the population of healthy subjects to generate a biomarkers score for each biomarker.

In some embodiments, the methods further comprise generating a composite score for the panel of biomarkers by comparing the level of each biomarker in the panel to a control or threshold value for each to generate a biomarkers score for each biomarker and combining the biomarkers scores to generate the composite score; and comparing the composite score to a composite threshold value, wherein a significant difference between the composite score to a composite threshold value is indicative of a impaired respiratory health.

In some embodiments, the control or threshold value for each biomarkers is based on a population average for healthy subjects.

In some embodiments, the biomarkers score for each biomarker in the panel is calculated by calculating the difference between the level of each biomarkers biomarker in the panel to a control or threshold value for each and dividing the difference by the standard deviation for the population of healthy subjects.

In some embodiments, the composite score is the sum or the average of the biomarkers scores for each biomarker in the panel.

Embodiments of the present disclosure also include methods of monitoring the lung health of a subject over time, comprising (a) assessing the lung health of the subject at a first timepoint by quantifying the levels of a panel of one or more biomarkers in a biological sample from the subject; (b) assessing the lung health of the subject at a second timepoint by quantifying the levels of a panel of one or more biomarkers in a biological sample from the subject; (c) comparing the levels of the panel of one or more biomarkers in the biological sample from the subject at the first timepoint to the second timepoint. In some embodiments, a significant difference between the level of one or more of the biomarkers from the first timepoint to the second timepoint is indicative of impaired respiratory health.

In some embodiments, the panel comprises 200 or fewer biomarkers.

In some embodiments, the panel comprises 20 or fewer biomarkers.

In some embodiments, the one or more biomarkers comprises a protein or a cDNA.

In some embodiments, the one or more biomarkers comprises a protein or a cDNA.

### BRIEF DESCRIPTION OF THE DRAWINGS

FIG. 1. Adapted lung function trajectories in CARDIA. These are adapted from previously developed and published trajectories of FEV1 percent predicted (Washko et al. 2020). However, for the purposes of this study, participants with FEV1 trajectories previously named “Ideal Lung Health,” “Preserved Good Lung Health,” and “Preserved Impaired Lung Health” were combined into one trajectory called “Normal decline” (normal peak with normal decline, N=2,332). The previously named “Worsening Lung Health” and “Persistently Poor Lung Health” were renamed “Accelerated decline” (normal peak with accelerated decline, N=138) and “Low peak” (N=46), respectively. Participants with “low peak” trajectory were excluded from analyses.

FIG. 2: Proteins in the susceptibility score and their function and/or associations with lung health and disease, ranked by lung-specific gene expression. Hierarchical clustering was generated using the Genotype-Tissue Expression (GTEx) Portal based on lung-specific gene expression measured in median transcripts per million (TPM). LASSO directionality refers to direction of their association with accelerated decline lung function trajectory in the LASSO model.

FIG. 3A-C. Mortality, incident lung disease, and incident respiratory exacerbation by quartile of proteomic respiratory susceptibility score. (A) Adjusted hazard ratio for all-cause death ascertained in all three cohorts (UK Biobank, COPDGene, CARDIA) and respiratory death in UK Biobank and COPDGene. (B) Adjusted hazard ratio for incident COPD in UK Biobank. (C) Adjusted odds ratio for one or more exacerbation and one more severe exacerbation. All analyses were adjusted for age, sex, self-identified race, body mass index (BMI), smoking status and pack-years at the time of proteomics measurement. In CARDIA and COPDGene, additional adjustment was made for FEV1 percent predicted and income. UK Biobank analyses were adjusted for self-report of current respiratory disease (COPD, emphysema, chronic bronchitis, asthma, or respiratory failure) and Townsend deprivation index. The Townsend deprivation index estimates deprivation within a census area and comprises four variables: unemployment, non-car ownership, non-home ownership, and household overcrowding. Analyses in COPDGene were additionally adjusted for platelet count and white blood cell count based on prior internal quality control.

FIG. 4. Cumulative incidence plots of all-cause mortality by quartile of proteomic respiratory susceptibility score. Unadjusted cumulative incidence of all-cause death by quartile of proteomic susceptibility score in the CARDIA (top), UK Biobank (middle), and COPDGene (bottom) cohorts. Shaded areas around each line represent the 95% confidence interval.

FIG. 5. Study design and timeline. Top panel: In the CARDIA cohort, we identified participants with normal decline and accelerated decline in lung function trajectories. These groups were adapted from previously published trajectory groups that were identified in CARDIA using up to six measurements of FEV1 percent predicted taken at years 0, 2, 5, 10, 20, and 30 (Washko et al 2020). Plasma samples drawn at year 25 were analyzed for large-scale protein identification (proteomics). It was then examined which circulating proteins were associated with an accelerated decline in FEV1 trajectory, independent of cross-sectional FEV1 at year 20. These proteins were used to derive the proteomic respiratory susceptibility score.” Bottom panel: The respiratory susceptibility score was applied to the COPDGene and UK Biobank cohort studies. Plasma samples drawn at “Visit 2” in COPDGene and the baseline visit in UK Biobank were analyzed for the proteins in the susceptibility score. The association between susceptibility score and incident COPD, respiratory exacerbations, respiratory death and all-cause death was examined. The median follow-up time for all-cause death was 6.5 years (IQR 4.9-7.5) in COPDGene and 13.7 years (IQR 12.0-14.5) in UK Biobank. Figure created with BioRender.com.

FIG. 6. Flowchart of participants in CARDIA cohort included in the analyses of proteins associated with an accelerated decline FEV1 trajectory. These participants were included in analyses examining associations between accelerated decline trajectory group and year 25 proteomics and were included in models deriving the susceptibility score.

FIG. 7. FEV1 percent predicted by exam year in the CARDIA cohort.

FIG. 8. Volcano plot of proteins associated with an Accelerated Decline lung function trajectory (versus Normal Decline). Magnitude and significance of associations between proteins at year 25 (mean age 50) and an Accelerated Decline trajectory in CARDIA. Associations are based on multivariable linear models adjusted for age, sex, center, and year 20 FEV1 percent predicted. Blue dots signify proteins with statistically significant associations at a 5% Benjamini-Hochberg false discovery rate (represented by dashed line). See Table 6 for details of significant protein associations.

FIG. 9. Results of the tissue-specific deconvolution of proteins significantly associated with an accelerated decline FEV1 trajectory in CARDIA. Tissue-specific gene expression activities of the proteins with significant positive associations with accelerated decline FEV1 trajectory were obtained from the Genotype-Tissue Expression (GTEx) database. Gene expression activities were scored by the R package singcore for each of the 37 tissues and ranked below. Proteins included were found to be significant in in multivariable regression models with Benjamini-Hochberg false discovery rate p-value <0.05.

FIG. 10. Gene ontology analysis of proteins associated with an accelerated decline FEV1 trajectory in CARDIA. Length of bar corresponds to the fold enrichment of each gene ontology term and the overlying number depicts the Benjamini-Hochberg false discovery rate p-value.

FIG. 11. Violin plot with box plot of susceptibility score distribution by lung function trajectory group in CARDIA. The median susceptibility score for the Normal Decline group was-0.15 (interquartile range 1.27) and the median susceptibility score for the Accelerated Decline group was 0.93 (interquartile range 1.22).

### Definitions

Unless otherwise defined, all technical and scientific terms used herein have the same meaning as commonly understood by one of ordinary skill in the art. The meaning and scope of the terms should be clear; in the event, however of any latent ambiguity, definitions provided herein take precedent over any dictionary or extrinsic definition. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Unless otherwise required by context, singular terms shall include pluralities and plural terms shall include the singular.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, macaque, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, “an individual is suspected of being susceptible at risk for accelerated decline” is meant to refer to an individual who is at an above-average risk of developing accelerated decline.

Examples of individuals at a particular risk of developing accelerated decline are those whose family medical history indicates above average incidence of accelerated decline, individuals of advanced age, individuals exhibiting signs or symptoms of MCI or SCI. Other factors which may contribute to an above-average risk of developing accelerated decline may be based upon an individual's specific genetic, medical, psychological, psychosocial, and/or behavioral background and characteristics.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “sample” is used in its broadest sense. For example, in some embodiments, it is meant to include a specimen (e.g., blood sample, cerebrospinal fluid (CSF) sample). In preferred embodiments, it is meant to include a biological sample. The present invention is not limited by the type of biological sample used or analyzed. The present invention is useful with a variety of biological samples including, but are not limited to, tissue (e.g., organ (e.g., heart, liver, brain, lung, stomach, intestine, spleen, kidney, pancreas, and reproductive (e.g., ovaries) organs; lung biopsy), glandular, skin, and muscle tissue), cell (e.g., blood cell (e.g., lymphocyte or erythrocyte), muscle cell, tumor cell, bronchial cell, bronchioalveolar cells, and skin cell), gas, bodily fluid (e.g., tracheal aspirate fluid, bronchoalveolar fluid, bronchoalveolar lavage sample, blood or portion thereof, serum, plasma, urine, semen, saliva, etc), or solid (e.g., stool) samples obtained from a human (e.g., adult, infant, or embryo) or animal (e.g., cattle, poultry, mouse, rat, dog, pig, cat, horse, and the like). Biological samples may be obtained from all of the various families of domestic animals, as well as feral or wild animals, including, but not limited to, such animals as ungulates, bear, fish, lagamorphs, rodents, etc. Biological samples also include biopsies and tissue sections (e.g., biopsy or section of tumor, growth, rash, infection, or paraffin-embedded sections), medical or hospital samples (e.g., including, but not limited to, bronchoalveolar lavage fluid (BAL) samples, tracheal aspirate fluid, blood samples, saliva, buccal swab, cerebrospinal fluid, pleural fluid, milk, colostrum, lymph, sputum, vomitus, bile, semen, oocytes, cervical cells, amniotic fluid, urine, stool, hair and sweat), and laboratory samples (e.g., subcellular fractions).

As used herein, the term “sensitivity” is defined as a statistical measure of performance of an assay (e.g., method, test), calculated by dividing the number of true positives by the sum of the true positives and the false negatives.

### DETAILED DESCRIPTION

Embodiments of the present disclosure provides include kits, compositions, and methods related to impaired respiratory health (e.g., diseases and conditions relating to lung injury). In particular, the present disclosure provides include kits, compositions, and methods for quantifying biomarkers (e.g., proteins, nucleic acids, etc.) associated with an accelerated decline in rapid forced expiratory volume in 1 s (FEV1) and assessing the risk of, monitoring, treating and/or preventing cardiovascular disease (CVD).

In experiments conducted during development of embodiments herein, data from three diverse cohorts with varied smoking histories were to characterize a proteomic risk score of increased respiratory susceptibility that was associated with future respiratory morbidity and mortality. A susceptibility score in the highest quartile was associated with a 54% higher odds of respiratory exacerbation requiring hospitalization, 4 times higher odds of incident COPD, and 3.6 to 6 times higher odds of respiratory mortality compared to the lowest quartile. Many of the proteins included in the score are highly expressed in lung tissue. Some proteins have well-established roles in lung health and immunity while others are novel and may hold new insights into respiratory health. This tool allows for the identification of individuals with increased susceptibility to future respiratory morbidity and mortality without requiring years of clinical observation.

The susceptibility score determined herein is associated with traditional risk factors for impaired respiratory health and chronic lung disease, including smoking, asthma, increased BMI, as well as social determinants of health such as measures of lower socioeconomic status and race (exposure to racism or racial health inequities). The susceptibility score is associated with FEV1 decline in CARDIA regardless of smoking status, including among never smokers, suggesting it may identify disease risk outside of tobacco alone. The susceptibility score is also associated with incident COPD, respiratory mortality, and all-cause mortality independent of smoking, age, sex, race, asthma diagnosis, BMI, and socioeconomic status. Furthermore, the proteomic risk score derived in a cohort at mean age 50 can predict respiratory outcomes in individuals aged 45 to 75 years old. This is indicated that the score is generalizable to an age range common for COPD diagnosis as well as to earlier ages when disease interception may be more feasible. While the proteomics score is independently associated with short-term FEV1 decline in COPDGene, in some embodiments its strength is instead in identifying respiratory susceptibility even in those without decades of longitudinal spirometry.

Embodiments of the present disclosure include kits and methods for identifying, selecting, and analyzing (e.g., quantifying) at least one (e.g., one or more) biomarker (e.g., a protein (e.g., an exogenous or endogenous biopolymeric structure composed of amino acids (e.g., antibodies, contractile proteins, enzymes, hormonal proteins, structural proteins, storage proteins, and transport proteins) and/or their genetic elements (e.g., genomes)))). In some embodiments, a biomarker is associated with a molecular function, a cellular component, and/or a biological process. In some embodiments, the level of at least one (e.g., one or more) prognostic biomarker in a sample from a subject is indicative/prognostic/diagnostic of a condition/outcome in the subject (e.g., a prognostic biomarker).

Experiments were conducted during development of embodiments herein to identify a panel of biomarkers that when quantified individually and/or collectively provide information about the health status of a subject (e.g., health and/or makeup of the subject's respiratory health) and/or a treatment course of action to maintain or enhance the health of the subject. In some embodiments, methods of identifying such biomarkers are within the scope herein.

In some embodiments, a method for measuring impaired respiratory health (e.g., diseases and conditions relating to lung injury (e.g., infection, cancer, blocked blood flow in the lung (e.g., pulmonary embolism), etc.)) includes thoracic CT scans (e.g., inspiratory volumetric CT scans) of a subject's chest and/or a histogram classifier (e.g., a local or a global histogram classifier).

In some embodiments, a method for predicting and/or assessing impaired respiratory health (e.g., diseases and conditions relating to lung injury (e.g., infection, cancer, blocked blood flow in the lung (e.g., pulmonary embolism), etc.)) includes a quantitative and responsive physiologic measure of airflow obstruction in a subject (e.g., FEV1).

In some embodiments, a biological sample (e.g., blood or plasma) from a subject is collected. In some embodiments, venous blood from a subject is collected in a collection vessel (e.g., EDTA tubes) and centrifuged and separated into plasma. In some embodiments, the venous blood from a subject is centrifuged and separated into plasma within 2 hours of collection. In some embodiments, the plasma is stored in aliquots (e.g., 0.5 ml) at −80° C.

In some embodiments, a method for identifying a biomarker includes an aptamer-based proteomics assay capable of measuring human protein analytes in serum, plasma, and other biological matrices with high sensitivity and specificity is used. In some embodiments, a chemically modified single-stranded DNA aptamer that binds to epitopes on a target protein and reagents are then hybridized to complementary sequences on a DNA microarray in the Assay to yield relative fluorescence units. In some embodiments, an aptamer-based proteomics assay is a SOMAscan assay. In some embodiments, an aliquot (e.g., 130 uL) of plasma is used for proteomics analysis. For additional information regarding aptamer-based proteomics assays, see, e.g., Candia, J., Cheung, F., Kotliarov, Y. et al. Assessment of Variability in the SOMAscan Assay. Sci Rep 7, 14248 (2017). https://doi.org/10.1038/s41598-017-14755-5; incorporated by reference in its entirety. In some embodiments, standard processes (e.g., plate hybridization, median signal normalization, plate scaling, and calibration) are performed independently on all samples. In some embodiments, protein levels are reported in relative fluorescent units (RFU) and natural log-transformed for analysis.

Embodiments of the present disclosure include methods of analyzing a biological sample (e.g., blood or plasma sample) from a subject to isolate and/or detect and/or determine (quantitatively) the levels of various biomarkers (e.g., protein biomarkers, nucleic acids encoding protein biomarkers, etc.).

In some embodiments, a biomarker (e.g., protein biomarkers, nucleic acids encoding protein biomarkers, etc.) screen is performed on a sample (e.g., biological sample) from a subject to identify, quantify, etc. various biomarkers present. In some embodiments, a biomarker (e.g., protein biomarkers, nucleic acids encoding protein biomarkers, etc.) screen is performed to detect and/or quantify as many biomarkers as are detectable by the methods used. In some embodiments, one or more key biomarkers (e.g., protein biomarkers identified herein, nucleic acids encoding protein biomarkers, etc.) are detected and/or quantified. In some embodiments, 2 or more biomarkers are detected and/or quantified (e.g., 2, 3, 4, 5, 6, 7, 8, 9, 10, 12, 15, 20, 25, 30, 35, 40, 45, 50, 75, 100, or more). In some embodiments, 100 or fewer biomarkers are detected and/or quantified (e.g., 100, 80, 60, 50, 40, 30, 20, 10, 5, or fewer). In some embodiments, 1-20 (e.g., 1, 2, 3, 4, 5, 6, 7, 8, 9, 10, 11, 12, 13, 14, 15, 16, 17, 18, 19, 20) of the biomarkers described herein are detected and/or quantified.

In some embodiments, provided herein are panels of biomarkers. In some embodiments, a biomarker panel comprises two or more proteins, nucleic acids (e.g., encoding proteins herein, cDNA (e.g., spanning an exon-exon junction, full-length, etc.), etc.). In some embodiments, a biomarker panel comprises 2 or more biomarkers (e.g., 2, 3, 4, 5, 6, 7, 8, 9, 10, 11, 12, 13, 14, 15, 16, 17, 18, 19, 20, 25, 30, 40, 50, 75, 100, 200, or more, or ranges or values therebetween). In some embodiments, a biomarker panel comprises 2 or more biomarkers selected from Table 4 and/or Table 5 (e.g., 2, 3, 4, 5, 6, 7, 8, 9, 10, 11, 12, 13, 14, 15, 16, 17, 18, 19, 20, or ranges or values therebetween). In some embodiments, a biomarker panel comprises 100 or fewer biomarkers (e.g., 100, 90, 80, 70, 60, 50, 40, 30, 25, 20, 19, 18, 17, 16, 15, 14, 13, 12, 11, 10, 9, 8, 7, 6, 5, 4, or ranges or values therebetween). In some embodiments, a biomarker panel comprises 20 or fewer biomarkers selected from Table 4 and/or Table 5 (e.g., 20, 19, 18, 17, 16, 15, 14, 13, 12, 11, 10, 9, 8, 7, 6, 5, 4, 3, 2, or ranges or values therebetween). In some embodiments, a panel of biomarkers is selected from one or more key biomarkers (e.g., protein biomarkers identified herein, nucleic acids encoding protein biomarkers, etc.). In some embodiments, 2 or more panels of biomarkers are detected and/or quantified (e.g., 2, 3, 4, 5, 6, 7, 8, 9, 10, 12, 15, 20, 25, 30, 35, 40, 45, 50, 75, 100, or more). In some embodiments, 100 or fewer panels of biomarkers are detected and/or quantified (e.g., 100, 80, 60, 50, 40, 30, 20, 10, 5, or fewer). In some embodiments, 1-20 (e.g., 1, 2, 3, 4, 5, 6, 7, 8, 9, 10, 11, 12, 13, 14, 15, 16, 17, 18, 19, 20) of the panels of biomarkers described herein are detected and/or quantified.

In some embodiments, the biomarkers herein are detected as proteins or other biomarkers (e.g., nucleic acids) in a sample from a subject. In some embodiments, any technique and/or instrumentation suitable for detecting/quantifying proteins or other biomarkers (e.g., nucleic acids) in a complex environment may find use in embodiments herein. In some embodiments, a point-of-care (POC) device is utilized for detecting/quantifying biomarkers in a complex environment. In some embodiments, a POC device allows for detection/quantification of biomarkers at a clinic, hospital, or other testing facility accessible or near to a patient, at reduced cost compared to traditional instruments, and in a short time span. In some embodiments, a POC device utilizes biosensors comprising antibodies, antibody, fragments, aptamers, etc. for binding to biomarkers and optical, fluorescent, luminescent, electrochemical, etc. detection for quantifying the protein biomarkers. Embodiments herein are not limited by the technique, POC or otherwise, used for quantification of the biomarkers. In some embodiments, detecting/quantifying biomarkers includes methods such as amplifying nucleic acids (e.g., PCR), protein separation (e.g., protein electrophoresis (e.g., sodium dodecyl sulfate polyacrylamide gel electrophoresis (SDS-PAGE)), variations of chromatic methods (e.g., laser diffraction, and chromatography (e.g., thin-layer chromatography (TLC), and column chromatography (e.g., high-performance liquid-based chromatography (HPLC), ultra-performance liquid-based chromatography (UPLC), ion exchange column chromatography, hydrophobic interaction column chromatography), size exclusion chromatography, and affinity chromatography))), and immunoblotting (e.g., dot blotting, quantitative enzyme-linked immunosorbent assays (ELISA), and Western blotting (e.g., Colorimetric blotting, Fluorometric blotting, and Chemiluminescent blotting)) and protein identification (e.g., Edman Degradation, Nuclear Magnetic Resonance (NMR) spectroscopy, and protein mass spectrometry (e.g., peptide mass fingerprinting and de novo peptide sequencing).

In some embodiments, the biomarkers herein are detected as nucleic acids encoding the protein biomarkers identified in the experiments conducted during development of embodiments herein. Exemplary methods for detecting the presence or absence of a nucleic acid biomarker (e.g., mRNA, DNA, etc.) include, but are not limited to, polymerase chain reaction (PCR)-based technologies (e.g., reverse transcription PCR (RT-PCR) and quantitative or real-time RT-PCR (RT-qPCR)). Other methods include microarray analysis, RNA sequencing (e.g., next-generation sequencing (NGS)), in situ hybridization, and Northern blot.

In some embodiments, biomarkers are detected at the nucleic acid level. For example, the presence or amount of biomarker nucleic acid (e.g., mRNA) in a sample is determined (e.g., to determine the presence or level of biomarker expression). Biomarker nucleic acid (e.g., RNA, amplified cDNA, etc.) may be detected/quantified using a variety of nucleic acid techniques known to those of ordinary skill in the art, including but not limited to nucleic acid sequencing, nucleic acid hybridization, nucleic acid amplification (e.g., by PCR, RT-PCR, qPCR, etc.), microarray, Southern and Northern blotting, sequencing, etc. Non-amplified or amplified nucleic acids can be detected by any conventional means. For example, in some embodiments, nucleic acids are detected by hybridization with a detectably labeled probe and measurement of the resulting hybrids. Nucleic acid detection reagents may be labeled (e.g., fluorescently) or unlabeled, and may by free in solution or immobilized (e.g., on a bead, well, surface, chip, etc.).

In some embodiments, a method for measuring impaired respiratory health (e.g., diseases and conditions relating to lung injury (e.g., infection, cancer, blocked blood flow in the lung (e.g., pulmonary embolism), etc.)) includes a quantitative and responsive physiologic measure of airflow obstruction in a subject (e.g., FEV1). In some embodiments, the measure of FEV1 is by spirometer machine.

In some embodiments, the biomarkers and/or panels thereof and/or levels thereof that are provided herein correlate with normal respiratory health (e.g., relative to the general healthy population). In some embodiments, biomarkers present within a range relative to a population average and/or reference range indicates normal (e.g., not atypically high, not atypically low) levels of respiratory health (e.g., lack of diseases and conditions relating to lung injury). In some embodiments, biomarkers that are beyond a threshold different (e.g., higher) than a population average and/or reference range indicates abnormal (e.g., atypically high, atypically low) levels of respiratory health (e.g., potential for diseases and conditions relating to lung injury).

Experiments conducted during development of embodiments herein demonstrate that the levels of various protein biomarkers (or nucleic acids encoding such biomarkers) present in biological samples (e.g., plasma or blood) from a subject correlate to respiratory health of the subject and are diagnostic of the health of the subject. In some embodiments, provided herein are panels of biomarkers (e.g., comprising one or more biomarkers selected from the group consisting of Table 4 and/or Table 5), the levels of which in biological samples (e.g., blood or plasma sample) from a subject correlate with the respiratory health of the subject. In some embodiments, increased differences in the levels of one or more biomarkers of a panel (e.g., comprising proteins selected from the group consisting of Table 4 and/or Table 5) from the population average, above/below a threshold, etc. it is indicative of an atypical respiratory health (e.g., diseases and conditions relating to lung injury).

In some embodiments, the presence of and/or levels of modification and/or remodeling of a number of all or a subset of the biomarkers in a biomarker panel (e.g., proteins contained in the pulmonary extracellular matrix (ECM) (e.g., collagens, elastin, fibrin, fibronectin, laminin, proteoglycans, and glycoproteins that compose the structural framework; the integrins and other cell adhesion receptors that create feedback connections between pulmonary cells and the ECM; growth factors, cytokines, and chemokines for which the ECM serves as a reservoir; and the metalloproteinases that regulate modification and degradation)) are correlated to the presence of specific lung injury of the subject. For example, if a biomarker or group of biomarkers are quantified as, beyond a threshold from the population average, outside of a reference range, etc., this may indicate increased likelihood of chronic lung diseases (e.g., pulmonary fibrosis and emphysema). In some embodiments, a number of all or a subset of the biomarkers in a biomarker panel are associated with various diseases and conditions relating to impaired respiratory health (e.g., accelerated decline FID1, lung progression of COPD and pulmonary fibrosis, progressive fibrosing interstitial lung disease (ILD), idiopathic pulmonary fibrosis (IPF), emphysema, cellular stress and senescence, and lung cancers).

In some embodiments, methods are provided of assessing and/or analyzing the levels of biomarkers in a biological sample (e.g., blood or plasma sample) from a subject. In some embodiments, the protein biomarkers in the panels herein and/or assessed herein include one or more biomarkers selected from the group consisting of Table 4 and/or Table 5.

In some embodiments, a panel of one or more biomarkers is selected from the group consisting of Table 4 and/or Table 5.

In some embodiments, the biomarkers in the panels herein and/or assessed herein include one or more biomarkers selected from the group consisting of a first panel of protein biomarkers. In some embodiments the first panel comprises: SLAM family member 7; Interleukin-36 alpha; Oxytocin-neurophysin 1; Fatty acid-binding protein, adipocyte; Axin-2; Interleukin-17 receptor E; Orphan sodium- and chloride-dependent neurotransmitter transporter NTT5; BPI fold-containing family B member 1; Galectin-3-binding protein; Nuclear distribution protein nudE-like 1; C-C motif chemokine 22; Liver-expressed antimicrobial peptide 2; Tissue-type plasminogen activator; Protein S100-A9; Marginal zone B- and B1-cell-specific protein; C-C motif chemokine 18; Piezo-type mechanosensitive ion channel component 1; Glutathione S-transferase A1; Ribonuclease K6; Amiloride-sensitive amine oxidase [copper-containing]; Gastrokine-2; Protocadherin gamma-C3; Macrophage scavenger receptor types I and II: Extracellular domain; Lipopolysaccharide-binding protein; C-C motif chemokine 3; Peroxidasin homolog; Scavenger receptor class B member 1; Ephrin type-B receptor 3; SLAM family member 8; Endothelial cell-derived lipase; Thrombospondin-2; Glycerol-3-phosphate dehydrogenase [NAD (+)], cytoplasmic; Estradiol 17-beta-dehydrogenase 1; Trafficking protein particle complex subunit 3; Serine protease HTRA1; Complement C3d fragment; Macrophage-capping protein; DnaJ homolog subfamily B member 9; Triggering receptor expressed on myeloid cells 2; Angiopoietin-2; Complement C3b, inactivated; Retinoic acid receptor responder protein 2; Complement component C9; Malignant T-cell-amplified sequence 1; Leukocyte immunoglobulin-like receptor subfamily A member 5; Glycerol-3-phosphate dehydrogenase [NAD (+)], cytoplasmic; Histone H2A type 1-A; Choline/ethanolamine kinase; Histone H2B type 1-K; Insulin; Myeloblastin; Growth hormone receptor; Serine/arginine-rich splicing factor 7; Interleukin-1 receptor antagonist protein; T-cell immunoglobulin and mucin domain-containing protein 4; Vesicular integral-membrane protein VIP36; V-set and transmembrane domain-containing protein 2-like protein; Transformer-2 protein homolog beta; E-selectin; Cytosolic Fe-S cluster assembly factor NUBP2; Ficolin-1; Gamma-glutamyl hydrolase; Leukocyte cell-derived chemotaxin-2; Inhibin beta A chain; Collagen alpha-3 (VI) chain: Bovine pancreatic trypsin inhibitor/Kunitz inhibitor domain, isoform 1; Marginal zone B- and B1-cell-specific protein; Regulator of G-protein signaling 4; C-C motif chemokine 21; Angiopoietin-2; Retinoblastoma-like protein 2; 5-hydroxytryptamine receptor 6; Protein S100-A12; Inhibin beta C chain; Bone sialoprotein 2; V-set and immunoglobulin domain-containing protein 4; Intercellular adhesion molecule 1; Triggering receptor expressed on myeloid cells 1; Collagen alpha-1 (XXVIII) chain; Olfactomedin-like protein 3; Tumor necrosis factor receptor superfamily member 10A; Serine/threonine-protein phosphatase 1 regulatory subunit 10; IGF-like family receptor 1; Thyroid transcription factor 1-associated protein 26; Tyrosine-protein phosphatase non-receptor type 7; G antigen 2; Replication initiator 1; ADAMTS-like protein 2; WAP four-disulfide core domain protein 2; Protein phosphatase 1 regulatory subunit 1A; PIH1 domain-containing protein 2; Rho GTPase-activating protein 36; 2-methoxy-6-polyprenyl-1,4-benzoquinol methylase, mitochondrial; Colipase-like protein 1; Sialic acid-binding Ig-like lectin 12: Ig-like C2-type 2 domain, Isoform short; Osteopetrosis-associated transmembrane protein 1; Cystatin B; Delta-like protein 1; Tumor necrosis factor receptor superfamily member 6; Insulin-like growth factor-binding protein 4; Lysozyme C; Protein FAM19A5; FXYD domain-containing ion transport regulator 6; Alpha-1-antichymotrypsin complex; Trafficking protein particle complex subunit 5; Protein FAM234B: N-term; Heat shock 70 kDa protein 1B; Cellular retinoic acid-binding protein 2; Z-DNA-binding protein 1; Transcriptional repressor protein YY1; Beta-2-microglobulin; Triggering receptor expressed on myeloid cells 2; Far upstream element-binding protein 2; Fc receptor-like protein 3; ATPase family AAA domain-containing protein 2; Galectin-9; Calcipressin-3; Plastin-2; Leucine-rich repeats and immunoglobulin-like domains protein 1; Complement factor B; Heat shock 70 kDa protein 1A; E3 ubiquitin-protein ligase RAD18; Syndecan-3; Antizyme inhibitor 1; Complement component C9; Ubiquitin-conjugating enzyme E2 G2; Interleukin-2; Holo-Transcobalamin-2; Ephrin-A1; Oncoprotein-induced transcript 3 protein; Stathmin-3; Tumor necrosis factor receptor superfamily member 1B; Asialoglycoprotein receptor 1; Methionine-R-sulfoxide reductase B1; CREB-binding protein; Serine/arginine-rich splicing factor 6; Synembryn-A; Myc target protein 1; Heparin cofactor 2; Ataxin-2-binding protein 1; GTPase IMAP family member 6; Galectin-4; Leukocyte immunoglobulin-like receptor subfamily A member 5; Thymidine kinase, cytosolic; ADP-ribosylation factor-like protein 15; DCN1-like protein 1; Coagulation factor IXab; Coagulation factor IX; Heat shock 70 kDa protein 1A; Tumor necrosis factor receptor superfamily member 1B; Sperm surface protein Sp17; Complement Clr subcomponent-like protein; Peptidyl-prolyl cis-trans isomerase C; Protein FAM204A; Interleukin-17C; Gamma-aminobutyric acid receptor-associated protein-like 1; Legumain; Inactive ribonuclease-like protein 10; Vitronectin; HLA class II histocompatibility antigen, DR beta 3 chain; Heat shock 70 kDa protein 1A; Tolloid-like protein 1; Macrophage receptor MARCO; Four and a half LIM domains protein 1; Gamma-aminobutyric acid receptor-associated protein; Killer cell immunoglobulin-like receptor 2DS4; Protein BUD31 homolog; Alpha-aminoadipic semialdehyde dehydrogenase; Inactive serine protease 35; Ubiquitin-conjugating enzyme E2 D2; Complement factor D; Dipeptidyl peptidase 1; Heat shock cognate 71 kDa protein; Antileukoproteinase; WD repeat-containing protein 5; Out at first protein homolog; Neuroblastoma suppressor of tumorigenicity 1; Tumor necrosis factor ligand superfamily member 11; Kynureninase; Ephrin-A4; EGF-containing fibulin-like extracellular matrix protein 1; Tumor necrosis factor receptor superfamily member 19L; Serum amyloid P-component; Complement C2; Follistatin-related protein 3; Cystatin-C; Serine/threonine-protein kinase 17B; Protein RIC-3; Leukocyte immunoglobulin-like receptor subfamily B member 4; Metalloproteinase inhibitor 1; C-X-C motif chemokine 16; Fibulin-5; Transmembrane gamma-carboxyglutamic acid protein 1: Cytoplasmic domain; C4b-binding protein alpha chain; Tumor necrosis factor ligand superfamily member 15; Secreted and transmembrane protein 1; Heat shock 70 kDa protein 1A; Complement Clq tumor necrosis factor-related protein 5; Ganglioside GM2 activator; Gamma-aminobutyric acid receptor-associated protein; DnaJ homolog subfamily C member 11; Atrial natriuretic factor; D-dimer; RecQ-mediated genome instability protein 1; von Willebrand factor A domain-containing protein 1; Pituitary adenylate cyclase-activating polypeptide 38; Uncharacterized protein C14orf93; Gem-associated protein 6; Matrix Gla protein; Guanine nucleotide exchange factor VAV3; Inactive dipeptidyl peptidase 10; Plastin-2; Fibrinogen gamma chain; Paraspeckle component 1; Calcium-dependent phospholipase A2; Complement Clq tumor necrosis factor-related protein 1; Integrin a5b1; Calpain-9; Cadherin-1; Complement factor H-related protein 1; Complement factor H; Antithrombin-III; Apolipoprotein C-I; Neurogenic locus notch homolog protein 1; Heat shock 70 kDa protein 12A; Kallistatin; Glutathione peroxidase 3; Kallistatin; Prostasin; Repulsive guidance molecule A; Serum albumin; Epidermal growth factor receptor; Protein SCO1 homolog, mitochondrial; Mitogen-activated protein kinase 6; B melanoma antigen 3; Heparan-sulfate 6-O-sulfotransferase 3; Platelet endothelial aggregation receptor 1: Extracellular domain; Neural cell adhesion molecule L1-like protein; Neuropeptide S; Ephrin type-A receptor 4; Beta-hexosaminidase subunit beta; RGM domain family member B; Leucine-rich repeats and immunoglobulin-like domains protein 3; Melanoma-associated antigen MUC18; Zinc finger protein 230; Inter-alpha-trypsin inhibitor heavy chain H2; Tetratricopeptide repeat protein 33; Leukemia inhibitory factor receptor; Glycosaminoglycan xylosylkinase; Methylglutaconyl-CoA hydratase, mitochondrial; Endothelial cell-selective adhesion molecule; Neurotrimin; MAD2L1-binding protein; Growth/differentiation factor 2; Glucosidase 2 subunit beta; Desmoglein-2; Contactin-1; Neural cell adhesion molecule L1-like protein; Inter-alpha-trypsin inhibitor heavy chain H1; Slit homolog 2 protein; Heat shock 70 kDa protein 1A; Sodium/potassium-transporting ATPase subunit beta-2; Speriolin-like protein; Leucine-rich repeat transmembrane neuronal protein 2; Endothelial cell-selective adhesion molecule; Mediator of RNA polymerase II transcription subunit 11; Anosmin-1; Microtubule-associated proteins 1A/1B light chain 3 beta 2; Apolipoprotein A-IV; Beta-1,4-galactosyltransferase 2; Neurotrimin; Interleukin-1 receptor type 1; Apolipoprotein D; Dihydrolipoyl dehydrogenase, mitochondrial; Gliomedin; Serine protease 57; Interleukin-19; Inactive tyrosine-protein kinase transmembrane receptor ROR1; Carbonyl reductase [NADPH] 3; Kunitz-type protease inhibitor 2; Endothelial cell-specific molecule 1; Bone morphogenetic protein receptor type-1A; 15 kDa selenoprotein; Complement Clq-like protein 3; von Willebrand factor A domain-containing protein 2; Anthrax toxin receptor 2; Semaphorin-3G; Tyrosine-protein kinase SYK: Protein kinase domain; Normal mucosa of esophagus-specific gene 1 protein; Ciliary neurotrophic factor receptor subunit alpha; SLIT and NTRK-like protein 4; Protocadherin-9; Uronyl 2-sulfotransferase; N-acetylglucosamine-1-phosphodiester alpha-N-acetylglucosaminidase; Tubulointerstitial nephritis antigen-like; Cystatin-M; Dickkopf-related protein 3; Neural cell adhesion molecule 2; Polypeptide N-acetylgalactosaminyltransferase 4; Ephrin type-A receptor 4; Vesicular, overexpressed in cancer, prosurvival protein 1; Dual specificity protein phosphatase 13 isoform A; Ectonucleotide pyrophosphatase/phosphodiesterase family member 5; NT-3 growth factor receptor; Endothelial cell-selective adhesion molecule; Dipeptidase 1; 3-mercaptopyruvate sulfurtransferase; Trafficking protein particle complex subunit 6B; Cell adhesion molecule 2; Anthrax toxin receptor 1; Dickkopf-related protein 4; Gamma-glutamyltransferase 5; Seizure 6-like protein; EMILIN-3: region 1; Klotho; Protocadherin-10: Extracellular domain; Transmembrane protein 132A; Syntaxin-1A; WAP, Kazal, immunoglobulin, Kunitz and NTR domain-containing protein 2; Interleukin-2 receptor subunit beta; Acetylcholinesterase; Contactin-2; Coiled-coil domain-containing protein 126; Alpha-amylase 2B; Integrin alpha V beta 3; WAP, Kazal, immunoglobulin, Kunitz and NTR domain-containing protein 2; Interleukin-1 Receptor accessory protein; Glyoxylate reductase/hydroxypyruvate reductase; Neural cell adhesion molecule 1, 120 kDa isoform; Secretogranin-3; Neural cell adhesion molecule 1; Netrin receptor UNC5D; Alpha-amylase 2B; Alpha-1,3-mannosyltransferase ALG2; Matrix-remodeling-associated protein 8: Extracellular domain; Receptor-type tyrosine-protein phosphatase delta; Interleukin-1 Receptor accessory protein; A disintegrin and metalloproteinase with thrombospondin motifs 13; Dickkopf-related protein 3; Gamma-enolase; Glypican-3; Kynurenine-oxoglutarate transaminase 3; Calcium and integrin-binding protein 1; Prostaglandin F2 receptor negative regulator; SLIT and NTRK-like protein 5; Activating signal cointegrator 1 complex subunit 2; DNA-directed RNA polymerases I, II, and III subunit RPABC4; Brevican core protein; Peroxisomal carnitine O-octanoyltransferase; Neuronal pentraxin receptor; Ciliary neurotrophic factor receptor subunit alpha; Netrin receptor UNC5D; Biglycan; Adhesion G protein-coupled receptor F5; SLIT and NTRK-like protein 1; Serine-rich single-pass membrane protein 1; IgLON family member 5; Voltage-dependent calcium channel subunit alpha-2/delta-3; Pancreatic alpha-amylase; Ephrin type-A receptor 6; Interleukin-35; Integrin αIIb1; Adiponectin; Fc receptor-like protein 4: Extracellular domain; Histone-lysine N-methyltransferase EHMT2; THAP domain-containing protein 4; Interleukin-27 subunit beta; Myelin-associated glycoprotein; Pancreatic triacylglycerol lipase; Cerebellin-2; Cysteine-rich with EGF-like domain protein 1; Ecto-ADP-ribosyltransferase 3; Ecto-ADP-ribosyltransferase 3; AP-1 complex-associated regulatory protein; Neuronal pentraxin receptor; Neurocan core protein; Cerebellin-1; Cystatin-SA; ADP-ribosylation factor 4; DCC; Amyloid-like protein 1; Pyruvate dehydrogenase E1 component subunit alpha, testis-specific form, mitochondrial; Advanced glycosylation end product-specific receptor, soluble; Galactosylgalactosylxylosylprotein 3-beta-glucuronosyltransferase 1; Oligodendrocyte-myelin glycoprotein; Lactase-phlorizin hydrolase; Insulin-like growth factor-binding protein 1; SLIT and NTRK-like protein 3; Carbonic anhydrase 6; and Carbonic anhydrase 6.

In some embodiments, the biomarkers in the panels herein and/or assessed herein include one or more biomarkers selected from the group consisting of a second panel of protein biomarkers. In some embodiments the second panel comprises: Nuclear distribution protein nudE-like 1; Ribonuclease pancreatic; Estradiol 17-beta-dehydrogenase 1; Phospholipase A2, membrane associated; Protein S100-A9; Triggering receptor expressed on myeloid cells 1; Ribonuclease K6; Cystatin B; Osteopetrosis-associated transmembrane protein 1; Delta-like protein 1; Retinoic acid receptor responder protein 2; 5-hydroxytryptamine receptor 6; 2-methoxy-6-polyprenyl-1,4-benzoquinol methylase, mitochondrial; Collagen alpha-1 (XXVIII) chain; Collagen alpha-3 (VI) chain: Bovine pancreatic trypsin inhibitor/Kunitz inhibitor domain, isoform 1; Corticotropin-releasing factor-binding protein; Complement Clr subcomponent-like protein; Gamma-aminobutyric acid receptor-associated protein; Gamma-aminobutyric acid receptor-associated protein-like 1; Serine/threonine-protein kinase 17B; Leukemia inhibitory factor receptor; Hedgehog-interacting protein; Semaphorin-3G; Protein FAM177A1; Neural cell adhesion molecule 1; SLIT and NTRK-like protein 4; Tubulointerstitial nephritis antigen-like; NT-3 growth factor receptor; Uronyl 2-sulfotransferase; Protocadherin-9; Seizure 6-like protein; Neurogenic locus notch homolog protein 2; WAP, Kazal, immunoglobulin, Kunitz and NTR domain-containing protein 2; Dickkopf-related protein 3; IgLON family member 5; Histatin-3; WAP, Kazal, immunoglobulin, Kunitz and NTR domain-containing protein 2; Brevican core protein; Cerebellin-2; Myelin-associated glycoprotein; Alpha-amylase 2B; Neurocan core protein; Mannan-binding lectin serine protease 1: Mannan-binding lectin serine protease 1 heavy chain; Oligodendrocyte-myelin glycoprotein; Cystatin-D; Amyloid-like protein 1; Carbonic anhydrase 6; and Carbonic anhydrase 6.

In some embodiments, cDNAs are generated from nucleic acids encoding the protein biomarkers identified herein. In some embodiments, full-length cDNAs are provided. In some embodiments, cDNAs are provided that span an exon-exon junction. In some embodiments, a panel of one or more cDNAs encode for one or more biomarkers selected from the group consisting of Table 4 and/or Table 5.

In some embodiments, the cDNAs in the panels herein and/or assessed herein encode for one or more biomarkers selected from the group consisting of the first panel of biomarkers and/or the second panel of biomarkers.

In some embodiments, the levels of one or more biomarkers is selected from the group consisting of Table 4 and/or Table 5 in a sample are quantitated. In some embodiments, an increased level (e.g., relative to a the population average, a reference range, control, or a threshold level) of one or more of the selected protein biomarkers in a biological sample (e.g., blood or plasma sample) from a subject is correlated with increased presence of impaired respiratory health.

In some embodiments, the level (e.g., concentration) of a biomarker is quantified in a sample from the subject using the methods, kits, reagents, etc. herein. In some embodiments, the level of the biomarker is compared to a control value that is representative of a “normal” level for a healthy subject. In some embodiments, the control value is the population average or a value based upon the population average or a reference range. In some embodiments, the difference between the quantified biomarker level and the control value is determined (e.g., quantified biomarker level minus the control value, absolute value of the quantified biomarker level, etc.). In some embodiments, the difference between the quantified biomarker level and the control value is divided by the standard deviation for the population used to determine the control value. In some embodiments, the difference between the quantified biomarker level and the control value divided by the standard deviation is the biomarker score for that given biomarker. In some embodiments, a biomarker score above a threshold value indicates an abnormal level for that protein. In some embodiments, a threshold for an individual biomarker score is 0.5, 1.0, 1.5, 2.0, 2.5, 3.0, 3.5, 4.0, 4.5, 5.0, or more, or ranges or values therebetween.

In some embodiments, a composite biomarker score is calculated by taking the sum of individual biomarker scores for a panel of biomarkers. In some embodiments, a composite biomarker score is calculated by taking the average of individual biomarker scores for a panel of biomarkers. In some embodiments, a composite biomarker score above a threshold value indicates an abnormal level for that biomarker. In some embodiments, when a composite biomarker score is an average of individual biomarker scores, a threshold for a composite biomarker score is 0.5, 1.0, 1.5, 2.0, 2.5, 3.0, 3.5, 4.0, 4.5, 5.0, or more, or ranges or values therebetween. In some embodiments, when a composite biomarker score is a sum of individual biomarker scores, a threshold for a composite biomarker score is the number of biomarkers in the panel multiplied by 0.5, 1.0, 1.5, 2.0, 2.5, 3.0, 3.5, 4.0, 4.5, 5.0, or more, or ranges or values therebetween.

In some embodiments, a subject is determined to have normal lung health (or a high likelihood of having normal lung health) if the composite biomarker score is below a composite threshold value. In some embodiments, the composite threshold value is n*(individual biomarker threshold), wherein n is the number of biomarkers analyzed in the panel and the individual biomarker threshold is 0.5, 0.75, 1.0, 1.25, 1.5, 1.75, 2.0, 2.5, 3.0, 3.5, 4.0, 4.5, 5.0, or more, or ranges or values therebetween.

In some embodiments, a kit is used to analyze a biomarker contained within a biological sample (e.g., blood or plasma sample). In some embodiments, the kit contains all components for quantifying a biomarker contained within a biological sample including at least one (e.g., one or more) biomarker (or a panel of biomarkers) that has a known level (e.g., a known concentration, a known weight, a known amount (e.g., a biomarker standard)). In some embodiments, the kit further contains at least one (e.g., one or more) analytical platforms (e.g., high-throughput platforms, automated platforms, etc.) utilizing a POC device, nuclear magnetic resonance (NMR) spectroscopy, gas chromatography (GC), liquid chromatography (LC), and/or mass spectrometry (MS) or NMR, GC, and/or LC coupled to MS.

Embodiments of the present disclosure also include methods of treatment (e.g., treatment with systemic steroids (e.g., synthetic derivatives of the natural steroid, cortisol, produced by the adrenal glands, and have profound anti-inflammatory effects), antibiotics (e.g., medicines that fight bacterial infections in people and animals), or both; pulmonary rehabilitation (e.g., a supervised medical program (e.g., as exercise stress test to measure oxygen level, blood pressure, and heart rate while exercising, a breathing test to determine how well the subject's lungs are working, a six-minute walk test to measure how far the subject can walk in six minutes, an education plan to learn how the lungs work, how to recognize symptoms of impaired respiratory health, how to quit smoking, how to conserve energy, how to avoid falling; a psychological counseling plan to learn how to cope with, reduce, or remove depression, anxiety, and other emotional problems; an exercise training plan to strengthen back, arms, legs, and muscles used to breath; medicine to open airways and/or improve oxygen uptake; nutritional counseling to learn how to prepare healthier meals and/or to improve weight-loss and/or weight maintenance and/or build muscle)).

### Experimental

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

Experiments were conducted during development of embodiments herein to identify circulating proteins associated with accelerated decline in FEV1 trajectory to derive a blood-based biomarker signature that would identify increased respiratory susceptibility through a single timepoint blood test. Proteomics, a large-scale sampling of proteins (in this case from plasma), provides a unique opportunity to molecularly identify individuals susceptible to lung disease, especially in the absence of longitudinal lung function data. A proteomic risk score of increased respiratory susceptibility was derived in the Coronary Artery Risk Development in Young Adults (CARDIA) cohort, a population-based cohort study with up to six lung function measurements over 30 years. Clinical relevance of this proteomic risk score was tested in a separate population-based cohort, UK Biobank (35,000 participants), and in an at-risk cohort of current and former smokers, the Genetic Epidemiology of COPD (COPDGene) Study (5,168 participants).

### Methods

### Study Design:

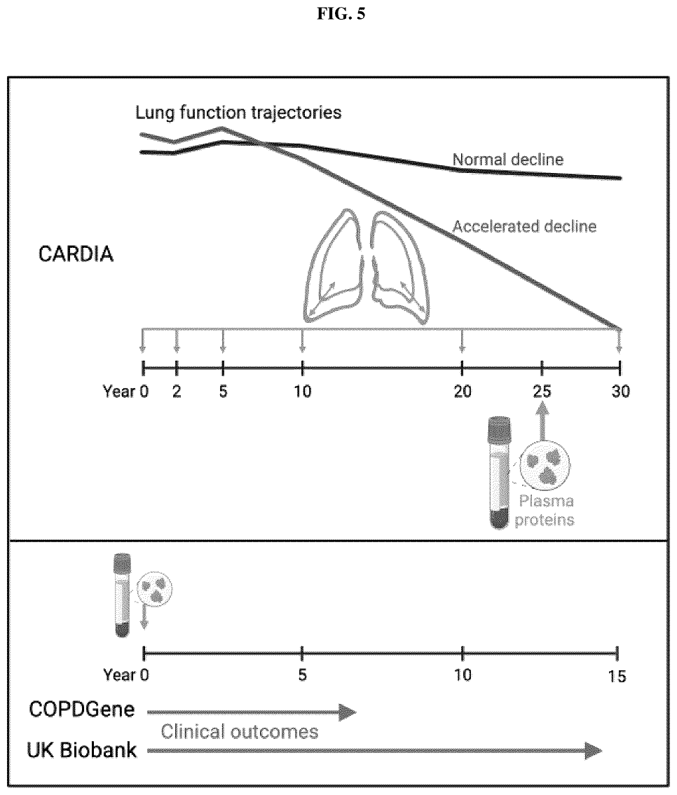

A prospective cohort study evaluating the association between a proteomic risk score of increased respiratory susceptibility and future respiratory disease, morbidity, and mortality. The susceptibility score was derived in the CARDIA cohort and applied in the UK Biobank (UKBB), and the Genetic Epidemiology of COPD (COPDGene) studies. The study design and timeline are depicted in FIG. 5. CARDIA and COPDGene are reviewed annually by institutional review boards at each center, and UKBB is monitored by an independent Ethics and Governance Council. All participants provided written informed consent.

### Study Populations:

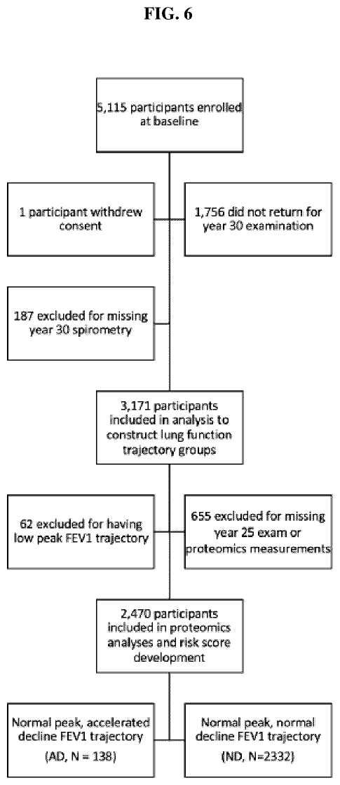

CARDIA study is a population-based cohort study of 5,115 adults, enrolled at age 18-30 from four centers in the United States. The susceptibility score was generated from and tested in 2,470 participants with complete data available at the year 25 follow-up visit (FIG. 6).

CARDIA is a prospective cohort study across four centers in the United States (Birmingham, AL, Chicago, IL, Minneapolis, MN and Oakland, CA) designed to identify cardiovascular disease risk factors for future among young adults. The flow chart of participants included in the CARDIA survival analysis is depicted in FIG. 6. 2,977 participants had year 25 proteomics data and were included in the unadjusted mortality analyses depicted in the cumulative incidence plots (FIG. 3). Of these participants, 409 had missing data on covariates and 2,568 were included in multivariable Cox regression survival analysis. The CARDIA study is reviewed annually by the institutional review boards at each participating institution and participants sign a new informed consent form at every examination.

UKBB is a population-based cohort study of 502,625 adults between 40 to 69 years old from the United Kingdom, of which 54,306 had some circulating proteomics data at the time of initial assessment. The proteomic susceptibility score was calculated for the 35,000 participants with complete proteomics data at baseline visit.

UK Biobank is a prospective cohort study of older adults from England, Scotland, and Wales. Proteomics assessments from blood samples collected during the initial assessment visit between 2006 and 2010 were run on 54,306 participants. There were 19,306 participants with some missing data for the proteins used in this study. This analysis includes the 35,000 participants with complete proteomics data to generate the susceptibility score. UK Biobank is approved by the North West Multi-center Research Ethics Committee as a Research Tissue Bank approval and the study is monitored by the independent UK Biobank Ethics and Governance Council. All UK Biobank subjects provided written informed consent. Analyses from UK Biobank were conducted under approved Research Proposal 57492.

COPDGene is a prospective study which represents a downstream at-risk cohort of 10,198 former and current smokers with at least a 10 pack-year smoking history, ages 45 to 80 years old. The proteomic susceptibility score was calculated for the 5,168 participants with complete proteomics data at COPDGene's “Visit 2” (considered the baseline visit for the present study).

COPDGene is a prospective cohort study with baseline visit in 2007-2012, when 10,198 participants aged 45-80 years with at least a 10 pack-year smoking history were recruited from 21 study centers in the United States (Phase 1). 5,268 participants had proteomics analyses performed on blood samples from Visit 2. 5,168 participants with complete covariate data were included in all-cause mortality analyses. 4,852 participants had adjudicated data on cause of death and were included in respiratory mortality analyses. COPDGene was approved by institutional review boards at each participating center, and all subjects provided written informed consent.

### FEV1 Trajectory Groups

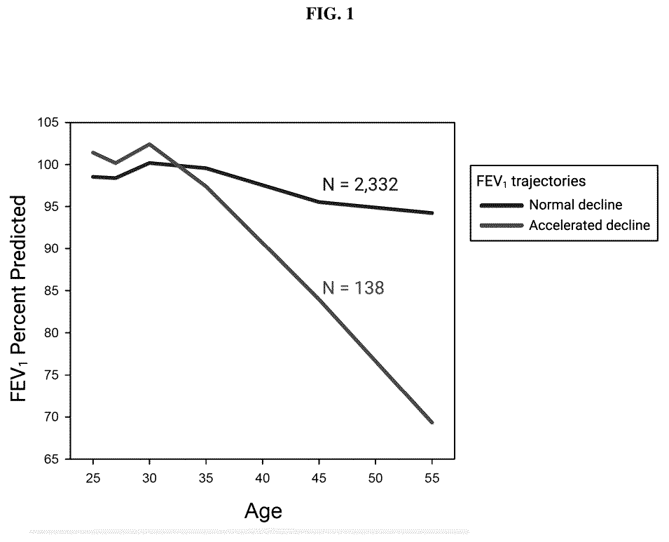

The FEV1 trajectory groups used in this study were accelerated decline (AD, with normal peak) and normal decline (ND, with normal peak) (FIG. 1). These were adapted from previously developed and published trajectories of FEV1 percent predicted.⁷ Longitudinal spirometry measurements (up to six timepoints over 30 years) were used to generate trajectories of FEV1 percent predicted using a group-based trajectory modeling approach (SAS PROC TRAJ). For the purposes of this study, participants with FEV1 trajectories previously named “Ideal,” “Preserved Good,” and “Preserved Impaired” Lung Health were combined into one trajectory called “Normal Decline” (normal peak with normal decline, N=2,332). The previously named “Worsening Lung Health” trajectory was renamed “Accelerated Decline” (normal peak with accelerated decline, N=138). Participants with “Persistently Poor Lung Health” trajectory (N=46) were excluded from analyses.

### Quantification of the Human Proteome

For CARDIA participants, plasma samples drawn at the year 25 visit were analyzed for protein identification using the SomaLogic SomaScan v4.1 assay, utilizing 7,335 SOMAmer aptamers to identify 6,609 unique human proteins. Proteomics identification was performed using the SomaScan v4.0 assay in COPDGene, as previously described¹², and the Olink Explore 1536 assay in UKBB. The initial analyses of individual proteins associated with AD trajectory were performed in CARDIA using all 7,335 aptamers. However, the susceptibility score was derived using the 1,082 unique human proteins that overlapped (by EntrezGeneID) across proteomics assays to facilitate analyses of clinical outcomes in the two validation cohorts. For SOMAmer aptamers that targeted the same protein at different epitopes, we used the aptamer with the highest correlation with the matching Olink protein based on published data, when available.¹³

### Outcomes

Outcomes of interest were incident COPD, respiratory exacerbations, respiratory death, and all-cause death. Incident COPD was only examined in UKBB given the large proportion of participants with prevalent COPD in COPDGene at the time of proteomics measurement. Data on respiratory exacerbations were only available in COPDGene. In COPDGene, participant vital status was monitored through regular outreach to participants or their designated proxies as well as through searches of the national death indexes. 14 In UKBB, death events were identified using death registry data and respiratory death and incident COPD were defined by Phecode/ICD-10 codes (Table 3).15 In COPDGene, respiratory deaths were adjudicated by two reviewers evaluating and classifying deaths as respiratory, cardiovascular, cancer, or other.¹⁶ Exacerbations were defined in COPDGene as an episode of increased cough, phlegm, or shortness of breath that lasted >48 hours and required treatment with systemic steroids, antibiotics, or both.¹⁷ Severe exacerbations were defined as an exacerbation requiring an emergency room visit or hospitalization. In COPDGene, we also examined rate of FEV1 decline, defined as difference in post-bronchodilator FEV1 percent predicted from “Visit 2” (time of proteomics measurement) to “Visit 3”, divided by 5 years. Lung function measurements were not available in this study's UKBB dataset.

### Statistical Analysis

### Proteomic Correlates of Accelerated Decline Trajectory

Multivariable logistic regression models in CARDIA was constructed to measure associations between each of the 7,335 SOMAmer protein aptamers (log 2-transformed) and AD trajectory (versus ND trajectory), adjusted for age, sex, center, and year 20 percent predicted FEV1 to identify proteins that are independent of important covariates. Adjustment for year 20 FEV1 was done to isolate the associations with lung function trajectory from the associations with cross-sectional lung function. Given the goal of discovering a protein signature that reflects respiratory susceptibility and encompasses the effects of traditional respiratory risk factors, rather than establishing a causal relationship between protein and FEV1 decline, minimal covariate adjustment was done at this stage. Type 1 error was controlled for using a false-discovery rate approach (FDR; Benjamini-Hochberg), with a 5% threshold. Gene ontology (GO)-based enrichment analysis for proteins passing 5% FDR was performed using the Database for Annotation, Visualization and Integrated Discovery (DAVID) platform.¹⁸ Ranked tissue-specific gene expression was examined for significant proteins using Genotype-Tissue Expression (GTEx) database¹⁹ and compared expression by each tissue type using the R package singscore.²⁰

### Proteomic Susceptibility Score Calculation

To derive susceptibility score in CARDIA, only the 1,082 proteins shared amongst the three cohorts were examined. A two-step reduction in the number of proteins included in the score was performed: (1) logistic regression to identify protein correlates of AD trajectory that would all be independent of important clinical covariates: age, sex, center, and year 20 percent predicted FEV1. (2) Proteins that were significantly associated with AD trajectory at an FDR 5% were included thereafter in LASSO modeling to identify a parsimonious group of proteins that are associated with AD trajectory. LASSO is a common supervised machine learning technique that removes collinear predictors and promotes sparsity within the model. LASSO was performed with five-fold cross-validation to optimize model hyperparameters. Proteins were log 2-transformed and scaled to zero mean and unit variance which is standard preprocessing for LASSO. The resulting LASSO model beta coefficients and intercept were used to generate a susceptibility score equation as follows: Susceptibility score=Intercept+(Coefficient₁*Protein₁ level)+(Coefficient₂*Protein₂ level)+ . . . + (Coefficientx*Proteinx level). The susceptibility score was calculated for each participant in the three cohorts, and it was used as a continuous variable and categorized into quartiles for subsequent analyses.

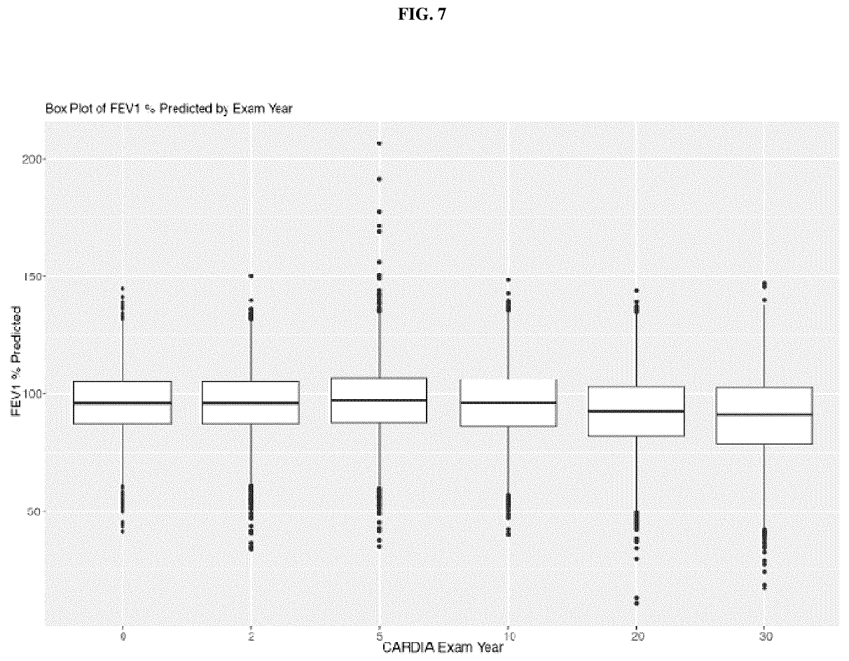

To examine whether the susceptibility score was reflective of FEV1 decline independent of tobacco smoke exposure, the CARDIA cohort was stratified by year 25 smoking status and used a linear mixed effects model to test the association between susceptibility score and yearly change in FEV1 percent predicted from year 5 to year 30, adjusted for age, sex, race, center, year 25 BMI, and year 25 smoking pack-years. Year 5 was chosen rather than year 0 given that in the CARDIA cohort, mean FEV1 percent predicted generally peaks at year 5 and declines linearly thereafter (FIG. 7).

### Clinical Outcomes

To test the association of the susceptibility score with clinical outcomes, time-to-event outcomes (all mortality, respiratory mortality, incident COPD) were modeled using multivariable Cox proportional hazards regression models, rate of FEV1 percent predicted decline using multivariable linear regression, odds of incident COPD exacerbation using multivariable logistic regressions, and frequency of COPD exacerbations using multivariable zero-inflated negative binomial regressions. Models were performed separately in each cohort. All models were complete-case analyses and adjusted for age, sex, self-identified race, body mass index (BMI), smoking status and pack-years at the time of proteomics. Given differences in available data across samples, we also adjusted for self-reported history of asthma and post-bronchodilator FEV1 percent predicted (COPDGene) or self-reported respiratory disease (UKBB) and for income (COPDGene) or Townsend deprivation index21 (UKBB). All-cause mortality was visualized across quartiles of the susceptibility score using Kaplan-Meier methods. In sensitivity analysis, the cohorts were restricted to only participants without lung disease at the time of proteomics measurement and examined the association between susceptibility score and all-cause mortality, respiratory mortality and respiratory exacerbations. In COPDGene this was defined by an FEV1/FVC >0.70 and FEV1 ≥80% predicted and in UKBB this was defined by self-reported or physician-diagnosed COPD, asthma, emphysema, chronic bronchitis, or respiratory failure.

### Spirometry and Lung Function Trajectories

Spirometry was measured in the CARDIA study at years 0, 2, 5, 10, 20 and 30 and followed standard procedures for calibration and maneuvers as recommended by the American Thoracic Society. To be included in the lung function trajectory analysis, participants had to have spirometry measurements from year 30 and at least one other time point. Previously defined adult trajectories of FEV1 percent predicted that were generated using a group-based trajectory modeling approach (SAS PROC TRAJ) were used. Although five trajectories were previously defined, given that the focus of this study was on differentiating participants with normal peak FEV1 and accelerated decline (AD) trajectory from those with normal peak FEV1 and normal decline (ND) trajectory, similar groups were combined (Ideal Lung Health, Good Lung Health, and Impaired Lung Health) in the ND trajectory group, and renamed Worsening Lung Health to “Accelerated decline” and Poor Lung Health to “Low peak” to result in 3 final FEV1 trajectory groups (FIG. 7). The “low peak” trajectory was excluded from analyses. To reinforce reproducibility, the previously defined FEV1 trajectories were used, given that the AD trajectory (aka “Worsening Lung Health”) had been previously associated with increased risk of incident obstruction and emphysema.

### Quantification of the Human Proteome

The SomaLogic SomaScan version version 4.1 assay used in CARDIA utilizes 7,335 SOMAmer aptamers to identify 6,609 unique human proteins. The SomaLogic SomaScan version 4.0 (5.0K) assay used in COPDGene uses 4,979 SOMAmer aptamers to identify 4,776 unique human proteins. The Olink Explore 1536 assay used in UK Biobank measures 1,472 protein analytes, capturing 1,463 unique proteins. The Olink assay uses Proximity Extension Assay, where a matched pair of antibodies labeled with unique complementary oligonucleotides bind to the respective target protein. In the UK Biobank proteomics analyses, 130 proteins were excluded due to a high proportion of measurements below the limit of detection (>40%) and four proteins were excluded for a high proportion of missing measurements (>10%). Plate hybridization, median signal normalization, plate scaling and calibration were performed on all samples. With the SomaScan assay, protein levels are reported in relative florescent units (RFU) and analyses were performed after log 2 transformation. In the Olink Explore 1536 assay, protein levels are reported as Normalized Protein expression (NPX), Olink's relative quantification unit on a log-2 scale. To facilitate analyses of clinical outcomes across all three cohorts, the susceptibility score was derived using only the 1,082 unique human proteins that overlapped (by EntrezGeneID) across proteomics platforms. Within the SomaScan assays, some proteins have more than one unique SOMAmer aptamer. For the Olink proteins which matched several SOMAmers, we used published data to choose the SOMAmer with the highest correlation with Olink. If there was no published correlation data, one SOMAmer was randomly selected for that protein.

### Results

Proteins Associated with an Accelerated Decline FEV1 Trajectory

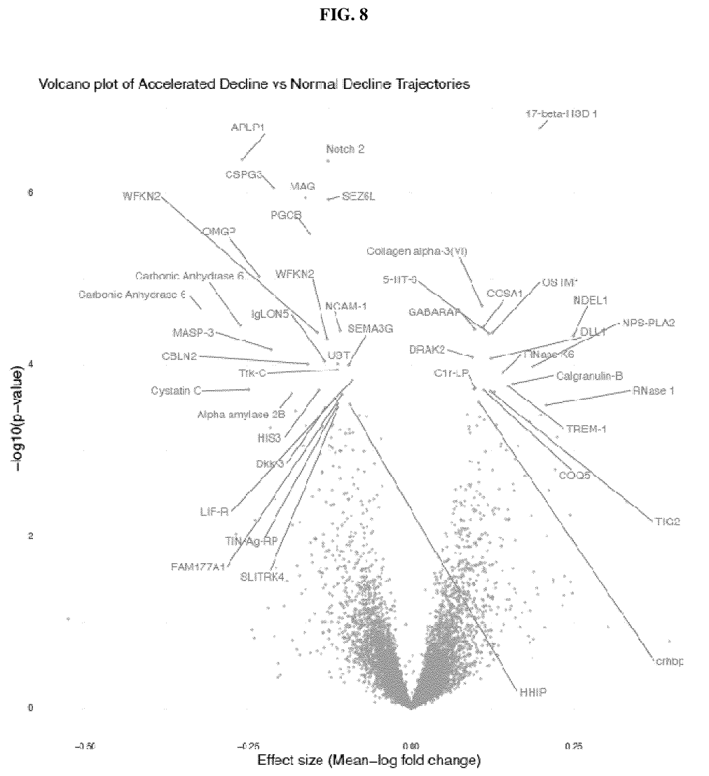

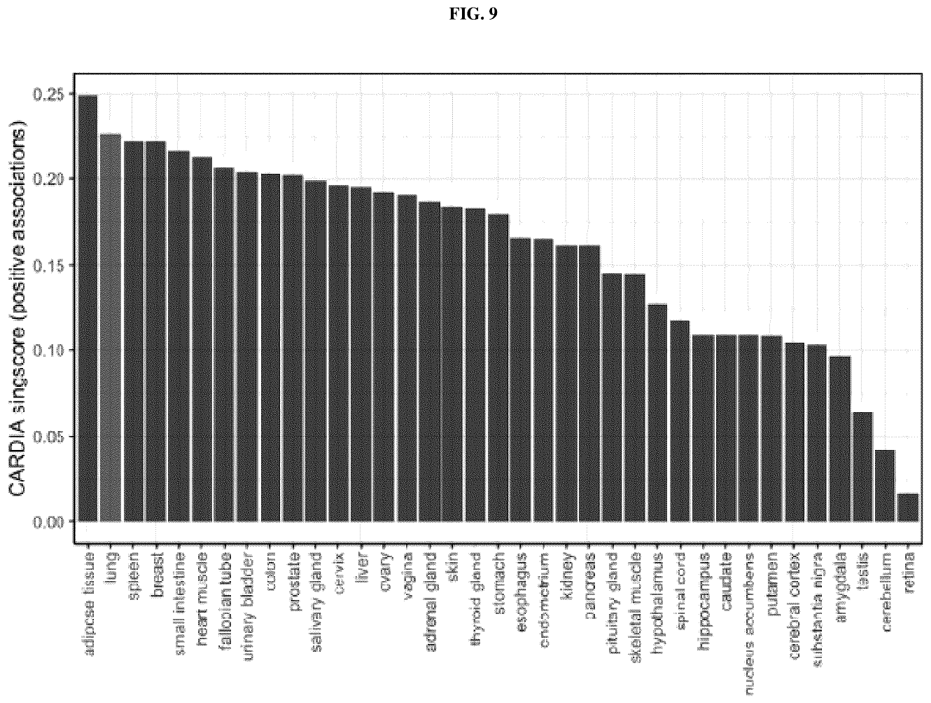

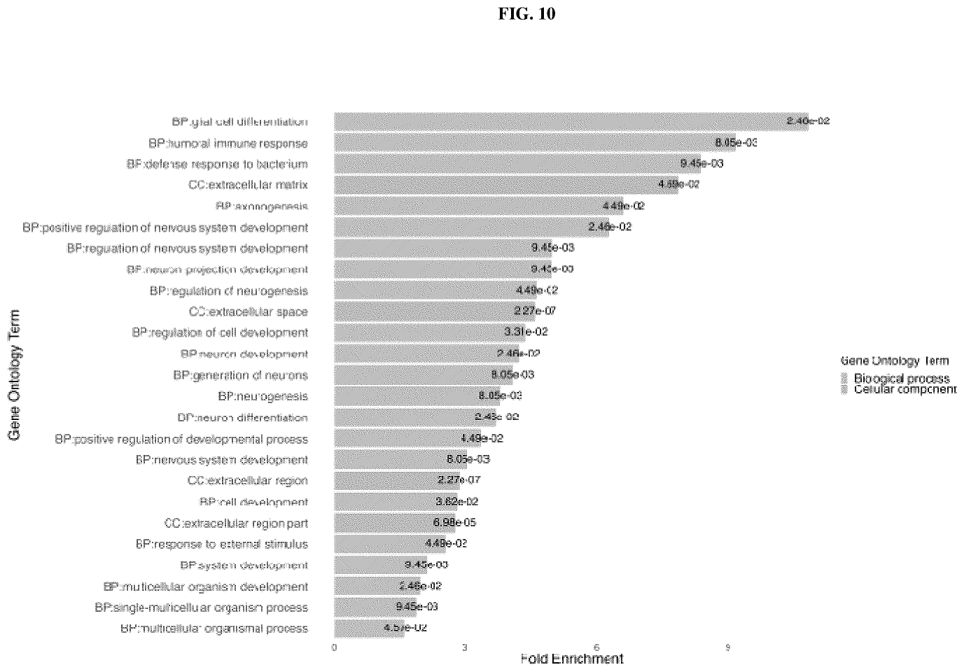

There were 138 participants with an AD trajectory and 2,332 participants with an ND trajectory in the CARDIA cohort (FIG. 1). The characteristics of the CARDIA participants with these trajectories are shown in Table 1. In logistic regression of AD versus ND trajectory, 413 proteins were significant in univariate analysis and 48 proteins (20 positively- and 28 negatively-associated with AD trajectory) were significant in multivariable analysis (FIG. 8, Tables 4-5). Analysis of the tissue-specific gene expression activity of the 20 proteins positively-associated with AD trajectory revealed that these proteins' gene transcripts were most commonly expressed in adipose and lung tissue (FIG. 9). Gene ontology analysis of the 48 proteins positively- and negatively-associated with AD trajectory demonstrated overrepresentation of proteins involved in extracellular components and in several different biological processes including humoral immune response and defense response to bacterium (FIG. 10).

TABLE 1
CARDIA cohort characteristics by FEV1 trajectory group

| | Normal | Accelerated |
|---|---|---|
| | decline (ND) | decline (AD) |
| Characteristic | trajectory | trajectory |
| N | 2,332 | 138 |
| Age, mean (SD) | 50 (4) | 49 (4) |
| Sex, n (%) | | |
| Male | 1,017 (44%) | 49 (36%) |
| Female | 1,315 (56%) | 89 (64%) |
| Race, n (%) | | |
| Black | 1,034 (44%) | 85 (62%) |
| White | 1,298 (56%) | 53 (38%) |
| Smoking status, n (%) | | |
| Never | 1,463/2,299 (64%) | 64/135 (47%) |
| Former | 523/2,299 (23%) | 21/135 (16%) |
| Current | 313/2,299 (14%) | 50/135 (37%) |
| Pack years, mean (SD) | 4.8 (9.5) | 9.4 (12.2) |
| BMI, mean (SD) | 29.9 (6.8) | 34.9 (9.4) |
| Baseline FEV1% | 97.7 (12.6) | 98.0 (13.0) |
| predicted, mean (SD) | | |
| Baseline FEV1/FVC | 83.2 (6.2) | 82.2 (6.5) |
| ratio, mean (SD) | | |
| Year 20 FEV1% | 94.1 (15.2) | 80.5 (16.5) |
| predicted, mean (SD) | | |
| Year 20 FEV1/FVC | 0.79 (0.06) | 0.75 (0.10) |
| ratio, mean (SD) | | |
| Year 30 FEV1% | 93.3 (16.2) | 66.8 (16.9) |
| predicted, mean (SD) | | |
| Year 30 FEV1/FVC | 0.77 (0.06) | 0.70 (0.12) |
| ratio, mean (SD) | | |
| Change in FEV1% | −4.3 (10.0) | −30.9 (12.3) |
| predicted*, mean (SD) | | |
| Change in FEV1 | −26.6 (11.2) | −50.4 (13.8) |
| (mL/year)^{†}, mean (SD) | | |

*Absolute change from year 0 to year 30.
^{†}Change per year from year 0 to year 30.
Characteristics from year 25 displayed, unless otherwise specified.

TABLE 4
Protein aptamer associations with accelerated decline FEV1 trajectory (vs normal decline trajectory) in univariate analyses.

| | | Entrez | | Log | | FDR |
|---|---|---|---|---|---|---|
| | | Gene | Aptamer | fold | p- | p- |
| Target Protein Name | UniProt ID | ID | ID | change | value | value |
| Zymogen granule membrane protein 16 | O60844 | 653808 | 821914 | 0.982289526 | 0.001657999 | 0.034846473 |
| C-reactive protein | P02741 | 1401 | 433749 | 0.679678346 | 2.59849E−10 | 9.52996E−07 |
| Leptin | P41159 | 3952 | 25755 | 0.537815982 | 6.47466E−07 | 0.000110446 |
| Leptin | P41159 | 3952 | 848424 | 0.497189464 | 7.9136E−06 | 0.00071662 |
| Serum amyloid A-1 protein | P0DJI8 | 6288 | 155152 | 0.460604659 | 2.50036E−06 | 0.000305669 |
| Alkaline phosphatase, placental type | P05187 | 250 | 78136 | 0.406595967 | 3.85241E−08 | 1.92331E−05 |
| Complement C3b | P01024 | 718 | 448059 | 0.403506432 | 3.2383E−06 | 0.000373901 |
| Fatty acid-binding protein, heart | P05413 | 2170 | 543763 | 0.340947555 | 4.9241E−09 | 4.51478E−06 |
| Cadherin-11: Extracellular domain | P55287 | 1009 | 1630510 | 0.338966242 | 3.7337E−05 | 0.002381351 |
| Phospholipase A2, membrane associated | P14555 | 5320 | 269274 | 0.301711096 | 2.2642E−10 | 9.52996E−07 |
| Transmembrane protein C1orf162 | Q8NEQ5 | 128346 | 68963 | 0.277710374 | 0.000157213 | 0.006627325 |
| Fatty acid-binding protein, adipocyte | P15090 | 2167 | 153867 | 0.275779528 | 8.48257E−07 | 0.000138266 |
| Prokineticin-2 | Q9HC23 | 60675 | 10754113 | 0.27564727 | 5.27793E−05 | 0.003104813 |
| BPI fold-containing family B member 1 | Q8TDL5 | 92747 | 112463 | 0.273206003 | 0.000101399 | 0.004829635 |
| Ribonuclease pancreatic | P07998 | 6035 | 72112 | 0.272520087 | 1.92478E−07 | 4.70609E−05 |
| CUB domain-containing protein 1 | Q9H5V8 | 64866 | 16818200 | 0.262308044 | 4.98218E−08 | 2.14966E−05 |
| Lysozyme g-like protein 1 | Q8N1E2 | 129530 | 924310 | 0.257973483 | 0.002287506 | 0.043405348 |
| Serum amyloid A-2 protein | P0DJI9 | 6289 | 1883265 | 0.251148362 | 9.61122E−05 | 0.004607731 |
| Amiloride-sensitive amine oxidase | P19801 | 26 | 15486126 | 0.246930284 | 0.002290098 | 0.043405348 |
| [copper-containing] | | | | | | |
| C-C motif chemokine 14 | Q16627 | 6358 | 290053 | 0.246346676 | 1.60708E−06 | 0.000226691 |
| C-C motif chemokine 22 | O00626 | 6367 | 229939 | 0.226270466 | 4.56997E−09 | 4.51478E−06 |
| Alkaline phosphatase, placental-like | P10696 | 251 | 671563 | 0.218469158 | 3.19803E−05 | 0.002113296 |
| Equatorin | Q9NQ60 | 54586 | 1116237 | 0.216662724 | 0.002535288 | 0.04614476 |
| Lymphoid-restricted membrane protein | Q12912 | 4033 | 1070491 | 0.215372026 | 0.000333544 | 0.011326613 |
| SLAM family member 7 | Q9NQ25 | 57823 | 54877 | 0.212489081 | 0.000507673 | 0.015451361 |
| Interleukin-36 alpha | Q9UHA7 | 27179 | 141507 | 0.211865614 | 0.002800208 | 0.049732513 |
| Oxytocin-neurophysin 1 | P01178 | 5020 | 835688 | 0.204673772 | 0.000120479 | 0.005523189 |
| Fatty acid-binding protein, adipocyte | P15090 | 2167 | 98519 | 0.201391538 | 6.11868E−06 | 0.000606494 |
| Axin-2 | Q9Y2T1 | 8313 | 842916 | 0.201255085 | 0.000158918 | 0.006660942 |
| Interleukin-17 receptor E | Q8NFR9 | 132014 | 2053568 | 0.200959137 | 1.74284E−05 | 0.001345653 |
| Orphan sodium- and chloride-dependent | Q9GZN6 | 28968 | 1305618 | 0.196589266 | 0.00214327 | 0.04181086 |
| neurotransmitter transporter NTT5 | | | | | | |
| BPI fold-containing family B member 1 | Q8TDL5 | 92747 | 1536738 | 0.194564587 | 0.001873078 | 0.037964123 |
| Galectin-3-binding protein | Q08380 | 3959 | 500052 | 0.194261632 | 0.00012704 | 0.005787804 |
| Nuclear distribution protein nudE-like 1 | Q9GZM8 | 81565 | 235456 | 0.192173415 | 0.000778499 | 0.020916813 |
| C-C motif chemokine 22 | O00626 | 6367 | 350878 | 0.191083749 | 6.073E−07 | 0.000106063 |
| Liver-expressed antimicrobial peptide 2 | Q969E1 | 116842 | 57081 | 0.187667625 | 6.88501E−05 | 0.003686242 |
| Tissue-type plasminogen activator | P00750 | 5327 | 221269 | 0.187463074 | 0.000144046 | 0.006237365 |
| Protein S100-A9 | P06702 | 6280 | 533949 | 0.185780756 | 1.38327E−06 | 0.000211381 |
| Marginal zone B- and B1-cell-specific | Q8WU39 | 51237 | 16322103044 | 0.185579131 | 1.00256E−05 | 0.000852278 |
| protein | | | | | | |
| C-C motif chemokine 18 | P55774 | 6362 | 3 | 0.184548922 | 2.2182E−06 | 0.000280525 |
| Piezo-type mechanosensitive ion channel | Q92508 | 9780 | 11838130 | 0.184056274 | 0.000300011 | 0.010630833 |
| component 1 | | | | | | |
| Glutathione S-transferase A1 | P08263 | 2938 | 171388 | 0.183961606 | 6.59686E−05 | 0.00355794 |
| Ribonuclease K6 | Q93091 | 6039 | 564620 | 0.183922555 | 2.35992E−08 | 1.57364E−05 |
| Amiloride-sensitive amine oxidase | P19801 | 26 | 62092 | 0.183686762 | 3.79447E−05 | 0.002381351 |
| [copper-containing] | | | | | | |
| Gastrokine-2 | Q86XP6 | 200504 | 64168 | 0.18328535 | 0.001268791 | 0.029828786 |
| Protocadherin gamma-C3 | Q9UN70 | 5098 | 785921 | 0.182790812 | 0.0001361 | 0.006013828 |
| Macrophage scavenger receptor types I | P21757 | 4481 | 1553397 | 0.181547016 | 3.25703E−05 | 0.002119601 |
| and II: Extracellular domain | | | | | | |
| Lipopolysaccharide-binding protein | P18428 | 3929 | 30746 | 0.180688393 | 4.70656E−05 | 0.002876885 |
| C-C motif chemokine 3 | P10147 | 6348 | 304059 | 0.178051204 | 7.04167E−05 | 0.003687169 |
| Peroxidasin homolog | Q92626 | 7837 | 134631 | 0.17721183 | 2.84923E−05 | 0.001995788 |
| Scavenger receptor class B member 1 | Q8WTV0 | 949 | 205346 | 0.175222414 | 9.23334E−05 | 0.004545405 |
| Ephrin type-B receptor 3 | P54753 | 2049 | 92207 | 0.173643118 | 0.002254154 | 0.043170282 |
| SLAM family member 8 | Q9P0V8 | 56833 | 899465 | 0.173495509 | 0.001524011 | 0.033072836 |
| Endothelial cell-derived lipase | Q9Y5X9 | 9388 | 23148100 | 0.172279181 | 3.8812E−06 | 0.000412588 |
| Thrombospondin-2 | P35442 | 7058 | 333933 | 0.170376201 | 0.000284085 | 0.010219697 |
| Glycerol-3-phosphate dehydrogenase | P21695 | 2819 | 1369751 | 0.169773433 | 0.00249153 | 0.045802683 |
| [NAD(+)], cytoplasmic | | | | | | |
| Estradiol 17-beta-dehydrogenase 1 | P14061 | 3292 | 47083 | 0.168806539 | 5.54322E−07 | 0.000106063 |
| Trafficking protein particle complex | O43617 | 27095 | 143371 | 0.167855669 | 9.17191E−08 | 2.92504E−05 |
| subunit 3 | | | | | | |
| Serine protease HTRA1 | Q92743 | 5654 | 1559447 | 0.16766148 | 8.6937E−10 | 1.59421E−06 |
| Complement C3d fragment | P01024 | 718 | 580324 | 0.167569243 | 1.28444E−05 | 0.001050966 |
| Macrophage-capping protein | P40121 | 822 | 496850 | 0.166165667 | 2.47031E−05 | 0.001811971 |
| DnaJ homolog subfamily B member 9 | Q9UBS3 | 4189 | 1121440 | 0.165597282 | 2.9461E−07 | 6.54839E−05 |
| Triggering receptor expressed on myeloid | Q9NZC2 | 54209 | 563566 | 0.164574752 | 0.000314565 | 0.010978707 |
| cells 2 | | | | | | |
| Angiopoietin-2 | O15123 | 285 | 26022 | 0.164537658 | 3.32608E−05 | 0.002140073 |
| Complement C3b, inactivated | P01024 | 718 | 26831 | 0.163980551 | 0.000163811 | 0.006788456 |
| Retinoic acid receptor responder protein | Q99969 | 5919 | 307962 | 0.161573557 | 9.6204E−08 | 2.94023E−05 |
| 2 | | | | | | |
| Complement component C9 | P02748 | 735 | 13722105 | 0.160620488 | 1.27951E−06 | 0.000199685 |
| Malignant T-cell-amplified sequence 1 | Q9ULC4 | 28985 | 124889 | 0.159797465 | 0.000142946 | 0.006237365 |
| Leukocyte immunoglobulin-like receptor | A6NI73 | 353514 | 778725 | 0.157681821 | 1.57023E−07 | 4.42986E−05 |
| subfamily A member 5 | | | | | | |
| Glycerol-3-phosphate dehydrogenase | P21695 | 2819 | 110811 | 0.153876603 | 0.000572475 | 0.016796415 |
| [NAD(+)], cytoplasmic | | | | | | |
| Histone H2A type 1-A | Q96QV6 | 221613 | 2240212 | 0.150879409 | 0.001364258 | 0.030790262 |
| Choline/ethanolamine kinase | Q9Y259 | 1120 | 13117232 | 0.150473998 | 0.000338709 | 0.011396482 |
| Histone H2B type 1-K | O60814 | 85236 | 2240313 | 0.149757545 | 0.001826296 | 0.037210791 |
| Insulin | P01308 | 3630 | 488356 | 0.149059075 | 0.000209675 | 0.008094566 |
| Myeloblastin | P24158 | 5657 | 351449 | 0.147852954 | 0.000862166 | 0.022425481 |
| Growth hormone receptor | P10912 | 2690 | 294858 | 0.146560821 | 0.000284229 | 0.010219697 |
| Serine/arginine-rich splicing factor 7 | Q16629 | 6432 | 1298712 | 0.14651962 | 4.0859E−08 | 1.92331E−05 |
| Interleukin-1 receptor antagonist protein | P18510 | 3557 | 535389 | 0.146343715 | 0.000397584 | 0.013019086 |
| T-cell immunoglobulin and mucin | Q96H15 | 91937 | 1544933 | 0.146330918 | 0.000134314 | 0.006007289 |
| domain-containing protein 4 | | | | | | |
| Vesicular integral-membrane protein | Q12907 | 10960 | 763830 | 0.14605935 | 3.70151E−06 | 0.000405232 |
| VIP36 | | | | | | |
| V-set and transmembrane domain- | Q96N03 | 128434 | 654960 | 0.14461911 | 0.001414999 | 0.031451569 |
| containing protein 2-like protein | | | | | | |
| Transformer-2 protein homolog beta | P62995 | 6434 | 1237373 | 0.142226905 | 0.000192213 | 0.007557693 |
| E-selectin | P16581 | 6401 | 34701 | 0.141048616 | 0.002078363 | 0.041021404 |
| Cytosolic Fe-S cluster assembly factor | Q9Y5Y2 | 10101 | 2186311 | 0.140374413 | 4.30931E−05 | 0.002656202 |
| NUBP2 | | | | | | |
| Ficolin-1 | O00602 | 2219 | 229617 | 0.139831649 | 0.00208847 | 0.041021404 |
| Gamma-glutamyl hydrolase | Q92820 | 8836 | 937069 | 0.139467983 | 1.28953E−05 | 0.001050966 |
| Leukocyte cell-derived chemotaxin-2 | O14960 | 3950 | 1676311 | 0.139445616 | 0.000695771 | 0.01911417 |
| Inhibin beta A chain | P08476 | 3624 | 137388 | 0.139261184 | 0.000411281 | 0.013289645 |
| Collagen alpha-3(VI) chain:Bovine | P12111 | 1293 | 1119631 | 0.138787419 | 4.00376E−09 | 4.51478E−06 |
| pancreatic trypsin inhibitor/Kunitz | | | | | | |
| inhibitor domain, isoform 1 | | | | | | |
| Marginal zone B- and B1-cell-specific | Q8WU39 | 51237 | 778034 | 0.138605805 | 3.26239E−06 | 0.000373901 |
| protein | | | | | | |
| Regulator of G-protein signaling 4 | P49798 | 5999 | 2302018 | 0.137828843 | 1.04049E−05 | 0.000867276 |
| C-C motif chemokine 21 | O00585 | 6366 | 251657 | 0.137637229 | 5.34811E−05 | 0.003104813 |
| Angiopoietin-2 | O15123 | 285 | 1366076 | 0.136896825 | 0.000315816 | 0.010978707 |
| Retinoblastoma-like protein 2 | Q08999 | 5934 | 135652 | 0.136879884 | 0.000441907 | 0.013971484 |
| 5-hydroxytryptamine receptor 6 | P50406 | 3362 | 135615 | 0.136158881 | 1.51281E−06 | 0.000224724 |
| Protein S100-A12 | P80511 | 6283 | 58526 | 0.134490229 | 0.000289983 | 0.010375741 |
| Inhibin beta C chain | P55103 | 3626 | 64082 | 0.133984571 | 6.32943E−05 | 0.003490706 |
| Bone sialoprotein 2 | P21815 | 3381 | 1402384 | 0.13352083 | 0.000271903 | 0.01000261 |
| V-set and immunoglobulin domain- | Q9Y279 | 11326 | 1767828 | 0.133263658 | 5.78799E−05 | 0.00326576 |
| containing protein 4 | | | | | | |
| Intercellular adhesion molecule 1 | P05362 | 3383 | 434210 | 0.13275724 | 0.002281913 | 0.043405348 |
| Triggering receptor expressed on myeloid | Q9NP99 | 54210 | 92661 | 0.132165213 | 0.000259855 | 0.009675309 |
| cells 1 | | | | | | |
| Collagen alpha-1(XXVIII) chain | Q2UY09 | 340267 | 107021 | 0.13100157 | 8.68573E−08 | 2.92504E−05 |
| Olfactomedin-like protein 3 | Q9NRN5 | 56944 | 866033 | 0.130386575 | 1.5625E−06 | 0.000224724 |
| Tumor necrosis factor receptor | O00220 | 8797 | 483275 | 0.130030367 | 0.000437176 | 0.013942107 |
| superfamily member 10A | | | | | | |
| Serine/threonine-protein phosphatase 1 | Q96QC0 | 5514 | 2253395 | 0.128171096 | 8.61152E−05 | 0.004326403 |
| regulatory subunit 10 | | | | | | |
| IGF-like family receptor 1 | Q9H665 | 79713 | 724416 | 0.128105682 | 0.001179643 | 0.028291506 |
| Thyroid transcription factor 1−associated | Q9P031 | 29080 | 22393130 | 0.126639803 | 0.000565569 | 0.016773745 |
| protein 26 | | | | | | |
| Tyrosine-protein phosphatase non- | P35236 | 5778 | 146886 | 0.126513275 | 0.002021922 | 0.040255107 |
| receptor type 7 | | | | | | |
| G antigen 2 | Q13066 | 2574 | 2541380 | 0.125761344 | 2.54639E−06 | 0.000306192 |
| Replication initiator 1 | Q9BWE0 | 29803 | 1355478 | 0.125549803 | 6.03832E−05 | 0.003381 |
| ADAMTS-like protein 2 | Q86TH1 | 9719 | 637962 | 0.124715059 | 2.91036E−05 | 0.001995788 |
| WAP four-disulfide core domain protein 2 | Q14508 | 10406 | 1138875 | 0.124161013 | 9.38921E−05 | 0.004589573 |
| Protein phosphatase 1 regulatory subunit | Q13522 | 5502 | 177064 | 0.124039246 | 5.87891E−06 | 0.00059071 |
| 1A | | | | | | |
| PIH1 domain-containing protein 2 | Q8WWB5 | 120379 | 2038121 | 0.123796134 | 0.000274261 | 0.01000261 |
| Rho GTPase-activating protein 36 | Q6ZRI8 | 158763 | 628978 | 0.123542162 | 7.65019E−06 | 0.000710306 |
| 2-methoxy-6-polyprenyl-1,4-benzoquinol | Q5HYK3 | 84274 | 2211230 | 0.122188464 | 1.39155E−05 | 0.001121653 |
| methylase, mitochondrial | | | | | | |
| Colipase-like protein 1 | A2RUU4 | 340204 | 95263 | 0.121977703 | 0.001043577 | 0.02586025 |
| Sialic acid-binding lg-like lectin 12:lg-like | Q96PQ1 | 89858 | 835226 | 0.119686911 | 9.08E−08 | 2.92504E−05 |
| C2-type 2 domain, Isoform short | | | | | | |
| Osteopetrosis-associated transmembrane | Q86WC4 | 28962 | 1935325 | 0.119389786 | 9.4404E−06 | 0.000824349 |
| protein 1 | | | | | | |
| Cystatin B | P04080 | 1476 | 1976813 | 0.118917821 | 0.000228748 | 0.008693592 |
| Delta-like protein 1 | O00548 | 28514 | 826443 | 0.117221209 | 2.94874E−05 | 0.002002683 |
| Tumor necrosis factor receptor | P25445 | 355 | 92187 | 0.117053085 | 1.80306E−05 | 0.001377649 |
| superfamily member 6 | | | | | | |
| Insulin-like growth factor-binding protein | P22692 | 3487 | 295057 | 0.115389297 | 3.83416E−06 | 0.000412588 |
| 4 | | | | | | |
| Lysozyme C | P61626 | 4069 | 492010 | 0.114707302 | 0.001618765 | 0.034416358 |
| Protein FAM19A5 | Q7Z5A7 | 25817 | 560992 | 0.113091909 | 0.000682789 | 0.018864899 |
| FXYD domain-containing ion transport | Q9H0Q3 | 53826 | 2541253 | 0.112246134 | 0.000146511 | 0.006247996 |
| regulator 6 | | | | | | |
| Alpha-1-antichymotrypsin complex | P01011 | 12 | 415311 | 0.111974226 | 0.000410626 | 0.013289645 |
| Trafficking protein particle complex | Q8IUR0 | 126003 | 8671378 | 0.111922574 | 0.000173393 | 0.007026729 |
| subunit 5 | | | | | | |
| Protein FAM234B:N-term | A2RU67 | 57613 | 7892132 | 0.111472573 | 0.001596038 | 0.034230825 |
| Heat shock 70 kDa protein 1B | P0DMV9 | 3303 | 1890126 | 0.110763253 | 7.03302E−05 | 0.003687169 |
| Cellular retinoic acid-binding protein 2 | P29373 | 1382 | 116967 | 0.110134548 | 0.001327539 | 0.030604523 |
| Z-DNA-binding protein 1 | Q9H171 | 81030 | 2283111 | 0.109762357 | 0.001517528 | 0.033072747 |
| Transcriptional repressor protein YY1 | P25490 | 7528 | 173371 | 0.109317536 | 0.002768677 | 0.049411797 |
| Beta-2-microglobulin | P61769 | 567 | 348528 | 0.109238497 | 0.00023706 | 0.008963069 |
| Triggering receptor expressed on myeloid | Q9NZC2 | 54209 | 1185121 | 0.108877974 | 0.002778373 | 0.049464482 |
| cells 2 | | | | | | |
| Far upstream element-binding protein 2 | Q92945 | 8570 | 249269 | 0.108722879 | 2.91137E−05 | 0.001995788 |
| Fc receptor-like protein 3 | Q96P31 | 115352 | 444015 | 0.108627096 | 0.000983199 | 0.024868151 |
| ATPase family AAA domain-containing | Q6PL18 | 29028 | 13043157 | 0.10755904 | 0.002203478 | 0.042757966 |
| protein 2 | | | | | | |
| Galectin-9 | O00182 | 3965 | 91974 | 0.107448379 | 4.87032E−05 | 0.002952378 |
| Calcipressin-3 | Q9UKA8 | 11123 | 2044212 | 0.107369467 | 0.002488093 | 0.045802683 |
| Plastin-2 | P13796 | 3936 | 9749190 | 0.107361428 | 1.21828E−07 | 3.57444E−05 |
| Leucine-rich repeats and | Q96JA1 | 26018 | 188316 | 0.107149201 | 0.000102867 | 0.004867916 |
| immunoglobulin-like domains protein 1 | | | | | | |
| Complement factor B | P00751 | 629 | 412972 | 0.106966821 | 6.24782E−10 | 1.52759E−06 |
| Heat shock 70 kDa protein 1A | P0DMV8 | 3303 | 1074918 | 0.106841885 | 0.000423071 | 0.013610624 |
| E3 ubiquitin-protein ligase RAD18 | Q9NS91 | 56852 | 2511124 | 0.106554005 | 0.000408931 | 0.013289645 |
| Syndecan-3 | O75056 | 9672 | 1661228 | 0.10620562 | 0.001744343 | 0.03578007 |
| Antizyme inhibitor 1 | O14977 | 51582 | 252826 | 0.105611591 | 0.000592424 | 0.017312481 |
| Complement component C9 | P02748 | 735 | 306043 | 0.10511556 | 0.000452617 | 0.014187815 |
| Ubiquitin-conjugating enzyme E2 G2 | P60604 | 7327 | 91996 | 0.105052587 | 9.96372E−06 | 0.000852278 |
| Interleukin-2 | P60568 | 3558 | 30701 | 0.105025266 | 0.000240401 | 0.008996647 |
| Holo-Transcobalamin-2 | P20062 | 6948 | 558421 | 0.104663886 | 0.000475083 | 0.014641725 |
| Ephrin-A1 | P20827 | 1942 | 20091138 | 0.104510808 | 0.000293003 | 0.010432889 |
| Oncoprotein-induced transcript 3 protein | Q8WWZ8 | 170392 | 629636 | 0.104161682 | 0.001180259 | 0.028291506 |
| Stathmin-3 | Q9NZ72 | 50861 | 801973 | 0.103788226 | 0.000805124 | 0.021417512 |
| Tumor necrosis factor receptor | P20333 | 7133 | 8368102 | 0.103222012 | 0.002081689 | 0.041021404 |
| superfamily member 1B | | | | | | |
| Asialoglycoprotein receptor 1 | P07306 | 432 | 545271 | 0.103162346 | 0.00048816 | 0.01498182 |
| Methionine-R-sulfoxide reductase B1 | Q9NZV6 | 51734 | 209711 | 0.103041823 | 6.10813E−05 | 0.003394176 |
| CREB-binding protein | Q92793 | 1387 | 136146 | 0.102785261 | 0.001335167 | 0.030604523 |
| Serine/arginine-rich splicing factor 6 | Q13247 | 6431 | 115733 | 0.102492553 | 0.001671464 | 0.03489802 |
| Synembryn-A | Q9NPQ8 | 60626 | 2524522 | 0.102228394 | 0.000328264 | 0.01125149 |
| Myc target protein 1 | Q8N699 | 80177 | 135411 | 0.101714793 | 0.001087914 | 0.02671209 |
| Heparin cofactor 2 | P05546 | 3053 | 331658 | 0.101570707 | 0.000176826 | 0.007087534 |
| Ataxin-2-binding protein 1 | Q9NWB1 | 54715 | 2207435 | 0.100748846 | 0.000626555 | 0.017952277 |
| GTPase IMAP family member 6 | Q6P9H5 | 474344 | 193027 | 0.098863673 | 0.000160186 | 0.00667593 |
| Galectin-4 | P56470 | 3960 | 2982828766 | 0.098681234 | 0.000363465 | 0.012173573 |
| Leukocyte immunoglobulin-like receptor | A6NI73 | 353514 | 29 | 0.098454306 | 0.000932472 | 0.023914981 |
| subfamily A member 5 | | | | | | |
| Thymidine kinase, cytosolic | P04183 | 7083 | 430158 | 0.098367686 | 0.000672888 | 0.018766665 |
| ADP-ribosylation factor-like protein 15 | Q9NXU5 | 54622 | 1841183 | 0.097204748 | 0.002510249 | 0.045802683 |
| DCN1-like protein 1 | Q96GG9 | 54165 | 173666 | 0.097199662 | 0.00050155 | 0.015328631 |
| Coagulation factor IXab | P00740 | 2158 | 530712 | 0.096572104 | 5.64284E−07 | 0.000106063 |
| Coagulation factor IX | P00740 | 2158 | 487632 | 0.096412626 | 1.71356E−05 | 0.001337123 |
| Heat shock 70 kDa protein 1A | P0DMV8 | 3303 | 1080322 | 0.096125364 | 0.001006749 | 0.025032225 |
| Tumor necrosis factor receptor | P20333 | 7133 | 315257 | 0.09610437 | 0.002242605 | 0.043061539 |
| superfamily member 1B | | | | | | |
| Sperm surface protein Sp17 | Q15506 | 53340 | 182428 | 0.095994155 | 0.002275919 | 0.043405348 |
| Complement C1r subcomponent-like | Q9NZP8 | 51279 | 93481 | 0.094866672 | 5.64826E−05 | 0.003236719 |
| protein | | | | | | |
| Peptidyl-prolyl cis-trans isomerase C | P45877 | 5480 | 1881921 | 0.094286593 | 0.000192106 | 0.007557693 |
| Protein FAM204A | Q9H8W3 | 63877 | 221406 | 0.093201577 | 0.000222753 | 0.008509878 |
| Interleukin-17C | Q9P0M4 | 27189 | 92555 | 0.093164859 | 0.00142153 | 0.031501284 |
| Gamma-aminobutyric acid receptor- | Q9H0R8 | 23710 | 1266144 | 0.093070059 | 0.000129131 | 0.005846785 |
| associated protein-like 1 | | | | | | |
| Legumain | Q99538 | 5641 | 362233 | 0.092826569 | 7.04317E−06 | 0.000679759 |
| Inactive ribonuclease-like protein 10 | Q5GAN6 | 338879 | 560262 | 0.0926 | 0.00163231 | 0.034604035 |
| 23849 | | | | | | |
| Vitronectin | P04004 | 7448 | 8280238 | 0.092521795 | 3.00637E−06 | 0.000355673 |
| HLA class Il histocompatibility antigen, DR | P79483 | 3125 | 69625 | 0.092338297 | 0.000811259 | 0.021482253 |
| beta 3 chain | | | | | | |
| Heat shock 70 kDa protein 1A | P0DMV8 | 3303 | 412424 | 0.092134987 | 1.01088E−05 | 0.000852278 |
| Tolloid-like protein 1 | O43897 | 7092 | 638390 | 0.091269293 | 0.000389738 | 0.012877161 |
| Macrophage receptor MARCO | Q9UEW3 | 8685 | 2013427 | 0.09106627 | 0.000937073 | 0.023949227 |
| Four and a half LIM domains protein 1 | Q13642 | 2273 | 1681413 | 0.090676587 | 0.001923534 | 0.038655136 |
| Gamma-aminobutyric acid receptor- | O95166 | 11337 | 17735130 | 0.090093461 | 9.57297E−05 | 0.004607731 |
| associated protein | | | | | | |
| Killer cell immunoglobulin-like receptor | P43632 | 3809 | 1715210 | 0.089407037 | 0.000645473 | 0.018280083 |
| 2DS4 | | | | | | |
| Protein BUD31 homolog | P41223 | 8896 | 2040119 | 0.088862925 | 0.000396469 | 0.013019086 |
| Alpha-aminoadipic semialdehyde | P49419 | 501 | 214983 | 0.087474431 | 0.001690787 | 0.034934987 |
| dehydrogenase | | | | | | |
| Inactive serine protease 35 | Q8N3Z0 | 167681 | 998397 | 0.086783986 | 0.001195844 | 0.028386772 |
| Ubiquitin-conjugating enzyme E2 D2 | P62837 | 7322 | 1884224 | 0.086629299 | 0.000617508 | 0.017767972 |
| Complement factor D | P00746 | 1675 | 294652 | 0.086120487 | 0.000214827 | 0.008250031 |
| Dipeptidyl peptidase 1 | P53634 | 1075 | 31785 | 0.085496432 | 0.000840091 | 0.022007385 |
| Heat shock cognate 71 kDa protein | P11142 | 3312 | 590391 | 0.084997934 | 0.002233248 | 0.043061539 |
| Antileukoproteinase | P03973 | 6590 | 44133 | 0.084754255 | 5.73476E−05 | 0.003260813 |
| WD repeat-containing protein 5 | P61964 | 11091 | 17760128 | 0.08396998 | 0.000135428 | 0.006013828 |
| Out at first protein homolog | Q86UD1 | 220323 | 64148 | 0.083327932 | 0.000554913 | 0.016613401 |
| Neuroblastoma suppressor of | P41271 | 100532736 | 294466 | 0.0831985 | 0.002675839 | 0.048343049 |
| tumorigenicity 1 | | | | | | |
| Tumor necrosis factor ligand superfamily | O14788 | 8600 | 1406148 | 0.0830451 | 0.002497921 | 0.045802683 |
| member 11 | | | | | | |
| Kynureninase | Q16719 | 8942 | 455964 | 0.08289822 | 0.001094994 | 0.02672025 |
| Ephrin-A4 | P52798 | 1945 | 1405061 | 0.082276839 | 0.000893944 | 0.02307667 |
| EGF-containing fibulin-like extracellular | Q12805 | 2202 | 848029 | 0.082076398 | 0.000470884 | 0.014610556 |
| matrix protein 1 | | | | | | |
| Tumor necrosis factor receptor | Q969Z4 | 84957 | 511531 | 0.081635591 | 0.001096496 | 0.02672025 |
| superfamily member 19L | | | | | | |
| Serum amyloid P-component | P02743 | 325 | 247454 | 0.08120143 | 0.001000337 | 0.024957387 |
| Complement C2 | P06681 | 717 | 31862 | 0.08119516 | 0.000752465 | 0.020374507 |
| Follistatin-related protein 3 | O95633 | 10272 | 343810 | 0.08118785 | 0.001650533 | 0.034789254 |
| Cystatin-C | P01034 | 1471 | 260959 | 0.079832038 | 0.000605092 | 0.017612513 |
| Serine/threonine-protein kinase 17B | O94768 | 9262 | 83996 | 0.079745016 | 0.000338521 | 0.011396482 |
| Protein RIC-3 | Q7Z5B4 | 79608 | 1122837 | 0.079555853 | 0.000144563 | 0.006237365 |
| Leukocyte immunoglobulin-like receptor | Q8NHJ6 | 11006 | 645370 | 0.079446737 | 0.000805894 | 0.021417512 |
| subfamily B member 4 | | | | | | |
| Metalloproteinase inhibitor 1 | P01033 | 7076 | 231733 | 0.079288954 | 0.000472079 | 0.014610556 |
| C-X-C motif chemokine 16 | Q9H2A7 | 58191 | 243649 | 0.079193652 | 0.000853401 | 0.022276511 |
| Fibulin-5 | Q9UBX5 | 10516 | 15585304 | 0.078776971 | 0.000174663 | 0.007039298 |
| Transmembrane gamma-carboxyglutamic | O14668 | 5638 | 830654 | 0.077582132 | 0.002328747 | 0.043798359 |
| acid protein 1:Cytoplasmic domain | | | | | | |
| C4b-binding protein alpha chain | P04003 | 722 | 9449150 | 0.077528565 | 0.001584125 | 0.034230825 |
| Tumor necrosis factor ligand superfamily | O95150 | 9966 | 296861 | 0.077456403 | 0.000827925 | 0.021766424 |
| member 15 | | | | | | |
| Secreted and transmembrane protein 1 | Q8WVN6 | 6398 | 130936 | 0.077414268 | 0.001104724 | 0.026831629 |
| Heat shock 70 kDa protein 1A | P0DMV8 | 3303 | 656378 | 0.077202475 | 0.002334882 | 0.043801429 |
| Complement C1q tumor necrosis factor- | Q9BXJ0 | 114902 | 781020 | 0.076855882 | 0.00111782 | 0.027060099 |
| related protein 5 | | | | | | |
| Ganglioside GM2 activator | P17900 | 2760 | 154416 | 0.076692994 | 0.001819483 | 0.037175239 |
| Gamma-aminobutyric acid receptor- | O95166 | 11337 | 2296620 | 0.073362247 | 0.001676516 | 0.03489802 |
| associated protein | | | | | | |
| DnaJ homolog subfamily C member 11 | Q9NVH1 | 55735 | 978375 | 0.073340366 | 0.00031952 | 0.01105508 |
| Atrial natriuretic factor | P01160 | 4878 | 544362 | 0.072488941 | 0.001191658 | 0.028 |
| | | | | | | 379268 |
| D-dimer | P02671 \| | 2243 \| | 490756 | 0.072313632 | 0.000105641 | 0.004941018 |
| | P02675 \| | 2244 \| | | | | |
| | P02679 | 2266 | | | | |
| RecQ-mediated genome instability | Q9H9A7 | 80010 | 139261 | 0.072180146 | 0.001515667 | 0.033072747 |
| protein 1 | | | | | | |
| von Willebrand factor A domain- | Q6PCB0 | 64856 | 638563 | 0.071747937 | 0.000636478 | 0.018165615 |
| containing protein 1 | | | | | | |
| Pituitary adenylate cyclase-activating | P18509 | 116 | 459422 | 0.071483874 | 0.00086915 | 0.022527266 |
| polypeptide 38 | | | | | | |
| Uncharacterized protein C14orf93 | Q9H972 | 60686 | 643959 | 0.070946514 | 0.001592249 | 0.034230825 |
| Gem-associated protein 6 | Q8WXD5 | 79833 | 216142 | 0.070586989 | 0.001594835 | 0.034230825 |
| Matrix Gla protein | P08493 | 4256 | 652087 | 0.070464898 | 0.001873621 | 0.037964123 |
| Guanine nucleotide exchange factor | Q9UKW4 | 10451 | 9830109 | 0.070402332 | 0.002023796 | 0.040255107 |
| VAV3 | | | | | | |
| Inactive dipeptidyl peptidase 10 | Q8N608 | 57628 | 789068 | 0.069980936 | 0.001460725 | 0.032175424 |
| Plastin-2 | P13796 | 3936 | 172311 | 0.069575391 | 0.000332664 | 0.011326613 |
| Fibrinogen gamma chain | P02679 | 2266 | 49897 | 0.067276723 | 0.000145411 | 0.006237365 |
| Paraspeckle component 1 | Q8WXF1 | 55269 | 2494879 | 0.065868872 | 0.0006177 | 0.017767972 |
| Calcium-dependent phospholipase A2 | P39877 | 5322 | 24491 | 0.062307555 | 0.002445802 | 0.045302926 |
| Complement C1q tumor necrosis factor- | Q9BXJ1 | 114897 | 63048 | 0.060774943 | 0.001166224 | 0.028138986 |
| related protein 1 | | | | | | |
| Integrin a5b1 | P08648 \| | 3678 \| | 219092 | 0.059365015 | 0.002400079 | 0.044681682 |
| | P05556 | 3688 | | | | |
| Calpain-9 | O14815 | 10753 | 2017339 | 0.058391974 | 0.002105793 | 0.041189304 |
| Cadherin-1 | P12830 | 999 | 14759149 | 0.053901305 | 0.002091616 | 0.041021404 |
| Complement factor H-related protein 1 | Q03591 | 3078 | 598250 | 0.051937184 | 0.000990915 | 0.024957387 |
| Complement factor H | P08603 | 3075 | 4159130 | 0.044242842 | 1.63076E−05 | 0.001300178 |
| Antithrombin-III | P01008 | 462 | 334460 | −0.049827026 | 5.3026E−05 | 0.003104813 |
| Apolipoprotein C-I | P02654 | 341 | 15364101 | −0.052760929 | 0.000192677 | 0.007557693 |
| Neurogenic locus notch homolog protein | P46531 | 4851 | 51077 | −0.056288266 | 7.87197E−05 | 0.004009786 |
| 1 | | | | | | |
| Heat shock 70 kDa protein 12A | O43301 | 259217 | 2526710 | −0.058970956 | 0.002542204 | 0.046156107 |
| Kallistatin | P29622 | 5267 | 344958 | −0.060962793 | 0.000275464 | 0.01000261 |
| Glutathione peroxidase 3 | P22352 | 2878 | 2179643 | −0.062087763 | 0.001328359 | 0.030604523 |
| Kallistatin | P29622 | 5267 | 141055 | −0.062120291 | 0.000139125 | 0.006110676 |
| Prostasin | Q16651 | 5652 | 62253 | −0.06385621 | 0.000764243 | 0.020609274 |
| Repulsive guidance molecule A | Q96B86 | 56963 | 54831 | −0.06448591 | 0.002325051 | 0.043798359 |
| Serum albumin | P02768 | 213 | 1838078 | −0.064989359 | 5.37575E−05 | 0.003104813 |
| Epidermal growth factor receptor | P00533 | 1956 | 26771 | −0.065285017 | 9.4482E−05 | 0.004589573 |
| Protein SCO1 homolog, mitochondrial | O75880 | 6341 | 785319 | −0.065928714 | 0.00129451 | 0.030336209 |
| Mitogen-activated protein kinase 6 | Q16659 | 5597 | 228583 | −0.067667128 | 0.001073849 | 0.026520819 |
| B melanoma antigen 3 | Q86Y29 | 85318 | 64426 | −0.067954 | 0.00089664 | 0.02307667 |
| Heparan-sulfate 6-O-sulfotransferase 3 | Q8IZP7 | 266722 | 1889623 | −0.068250052 | 0.001642764 | 0.034725276 |
| Platelet endothelial aggregation receptor | Q5VY43 | 375033 | 827531 | −0.068432024 | 0.00031168 | 0.010938631 |
| 1:Extracellular domain | | | | | | |
| Neural cell adhesion molecule L1-like | O00533 | 10752 | 895851 | −0.070318957 | 7.13808E−05 | 0.003687169 |
| protein | | | | | | |
| Neuropeptide S | P0C0P6 | 594857 | 639018 | −0.070662989 | 9.20393E−05 | 0.004545405 |
| Ephrin type-A receptor 4 | P54764 | 2043 | 1628817 | −0.07144446 | 0.000669136 | 0.018766665 |
| Beta-hexosaminidase subunit beta | P07686 | 3074 | 607561 | −0.072069762 | 8.109E−05 | 0.004102037 |
| RGM domain family member B | Q6NW40 | 285704 | 33318 | −0.072278696 | 0.000569488 | 0.01677589 |
| Leucine-rich repeats and | Q6UXM1 | 121227 | 332252 | −0.072535068 | 0.001332206 | 0.030604523 |
| immunoglobulin-like domains protein 3 | | | | | | |
| Melanoma-associated antigen MUC18 | P43121 | 4162 | 205122 | −0.07507947 | 0.001359236 | 0.030790262 |
| Zinc finger protein 230 | Q9UIE0 | 7773 | 2283839 | −0.075453606 | 0.000239058 | 0.00899224 |
| Inter-alpha-trypsin inhibitor heavy chain | P19823 | 3698 | 932633 | −0.07583251 | 2.39324E−06 | 0.000297533 |
| H2 | | | | | | |
| Tetratricopeptide repeat protein 33 | Q6PID6 | 23548 | 2041152 | −0.076876659 | 0.002708655 | 0.048696042 |
| Leukemia inhibitory factor receptor | P42702 | 3977 | 583749 | −0.077996963 | 0.000787532 | 0.021082279 |
| Glycosaminoglycan xylosylkinase | O75063 | 9917 | 7198197 | −0.078018446 | 6.93829E−05 | 0.003687169 |
| Methylglutaconyl-CoA hydratase, | Q13825 | 549 | 20931156 | −0.081477889 | 0.00224171 | 0.043061539 |
| mitochondrial | | | | | | |
| Endothelial cell-selective adhesion | Q96AP7 | 90952 | 29819 | −0.081865843 | 0.001746321 | 0.03578007 |
| molecule | | | | | | |
| Neurotrimin | Q9P121 | 50863 | 2055038 | −0.082921877 | 0.001249306 | 0.02946515 |
| MAD2L1-binding protein | Q15013 | 9587 | 2112995 | −0.083536553 | 0.000998771 | 0.024957387 |
| Growth/differentiation factor 2 | Q9UK05 | 2658 | 488021 | −0.083870713 | 0.00135236 | 0.030790262 |
| Glucosidase 2 subunit beta | P14314 | 5589 | 56875 | −0.084468722 | 0.000195273 | 0.007618765 |
| Desmoglein-2 | Q14126 | 1829 | 948475 | −0.084746543 | 0.002393088 | 0.044681682 |
| Contactin-1 | Q12860 | 1272 | 297461 | −0.085060852 | 0.000308535 | 0.010880299 |
| Neural cell adhesion molecule L1-like | O00533 | 10752 | 360154 | −0.08717334 | 0.000551259 | 0.016574754 |
| protein | | | | | | |
| Inter-alpha-trypsin inhibitor heavy chain | P19827 | 3697 | 7955195 | −0.088480935 | 7.46298E−07 | 0.000124411 |
| H1 | | | | | | |
| Slit homolog 2 protein | O94813 | 9353 | 1893028 | −0.089297116 | 0.000964036 | 0.024467834 |
| Heat shock 70 kDa protein 1A | P0DMV8 | 3303 | 142371 | −0.090277634 | 3.26537E−05 | 0.002119601 |
| Sodium/potassium-transporting ATPase | P14415 | 482 | 721887 | −0.092374526 | 0.001665416 | 0.03489802 |
| subunit beta-2 | | | | | | |
| Speriolin-like protein | Q9H0A9 | 84221 | 232455 | −0.092811396 | 0.00017972 | 0.007164385 |
| Leucine-rich repeat transmembrane | O43300 | 26045 | 690414 | −0.093910516 | 0.001519498 | 0.033072747 |
| neuronal protein 2 | | | | | | |
| Endothelial cell-selective adhesion | Q96AP7 | 90952 | 2053611 | −0.094580619 | 0.002463441 | 0.045514705 |
| molecule | | | | | | |
| Mediator of RNA polymerase II | Q9P086 | 400569 | 225009 | −0.094882996 | 0.001885394 | 0.037992767 |
| transcription subunit 11 | | | | | | |
| Anosmin-1 | P23352 | 3730 | 660318 | −0.095543771 | 0.002505039 | 0.045802683 |
| Microtubule-associated proteins 1A/1B | A6NCE7 | 643246 | 2096518 | −0.097385135 | 0.001362627 | 0.030790262 |
| light chain 3 beta 2 | | | | | | |
| Apolipoprotein A-IV | P06727 | 337 | 176859 | −0.098118898 | 0.001326325 | 0.030604523 |
| Beta-1,4-galactosyltransferase 2 | O60909 | 8704 | 959511 | −0.098181374 | 2.01301E−06 | 0.000263668 |
| Neurotrimin | Q9P121 | 50863 | 10907116 | −0.098471777 | 0.00139608 | 0.031125362 |
| Interleukin-1 receptor type 1 | P14778 | 3554 | 29919 | −0.099008679 | 0.001681508 | 0.03489802 |
| Apolipoprotein D | P05090 | 347 | 826220 | −0.09963152 | 0.000567129 | 0.016773745 |
| Dihydrolipoyl dehydrogenase, | P09622 | 1738 | 100251 | −0.100057678 | 4.96325E−05 | 0.002984051 |
| mitochondrial | | | | | | |
| Gliomedin | Q6ZMI3 | 342035 | 2059148 | −0.100848776 | 0.000678785 | 0.018859429 |
| Serine protease 57 | Q6UWY2 | 400668 | 835117 | −0.100982714 | 0.002660771 | 0.048189525 |
| Interleukin-19 | Q9UHD0 | 29949 | 303580 | −0.101028581 | 0.000171828 | 0.007026729 |
| Inactive tyrosine-protein kinase | Q01973 | 4919 | 259069 | −0.101165527 | 0.000639931 | 0.018193387 |
| transmembrane receptor ROR1 | | | | | | |
| Carbonyl reductase [NADPH] 3 | O75828 | 874 | 1409142 | −0.102844468 | 0.002025104 | 0.040255107 |
| Kunitz-type protease inhibitor 2 | O43291 | 10653 | 284313 | −0.104330857 | 0.002699694 | 0.048654193 |
| Endothelial cell-specific molecule 1 | Q9NQ30 | 11082 | 380516 | −0.105761462 | 0.000742158 | 0.020236924 |
| Bone morphogenetic protein receptor | P36894 | 657 | 48596 | −0.106293665 | 0.002030864 | 0.040260499 |
| type-1A | | | | | | |
| 15 kDa selenoprotein | O60613 | 9403 | 200873 | −0.106337633 | 3.79847E−05 | 0.002381351 |
| Complement C1q-like protein 3 | Q5VWW1 | 389941 | 2170715 | −0.109031946 | 0.000958406 | 0.024409391 |
| von Willebrand factor A domain- | Q5GFL6 | 340706 | 71289 | −0.109451573 | 0.0022291 | 0.043061539 |
| containing protein 2 | | | | | | |
| Anthrax toxin receptor 2 | P58335 | 118429 | 155595 | −0.110169199 | 0.001609549 | 0.034412242 |
| Semaphorin-3G | Q9NS98 | 56920 | 562821 | −0.111170878 | 1.53202E−06 | 0.000224724 |
| Tyrosine-protein kinase SYK: Protein | P43405 | 6850 | 1072213 | −0.111747357 | 0.001204948 | 0.028510633 |
| kinase domain | | | | | | |
| Normal mucosa of esophagus-specific | Q9C002 | 84419 | 64063 | −0.111824497 | 0.001729399 | 0.035632423 |
| gene 1 protein | | | | | | |
| Ciliary neurotrophic factor receptor | P26992 | 1271 | 141012 | −0.112038696 | 3.55307E−06 | 0.000400951 |
| subunit alpha | | | | | | |
| SLIT and NTRK-like protein 4 | Q8IW52 | 139065 | 713914 | −0.112113563 | 0.000607521 | 0.017613296 |
| Protocadherin-9 | Q9HC56 | 5101 | 1055826 | −0.112117324 | 0.000168984 | 0.006963485 |
| Uronyl 2-sulfotransferase | Q9Y2C2 | 10090 | 836474 | −0.112705846 | 2.61491E−05 | 0.001899047 |
| N-acetylglucosamine-1-phosphodiester | Q9UK23 | 51172 | 1120815 | −0.112800742 | 7.61285E−06 | 0.000710306 |
| alpha-N-acetylglucosaminidase | | | | | | |
| Tubulointerstitial nephritis antigen-like | Q9GZM7 | 64129 | 11192168 | −0.115247256 | 7.82445E−05 | 0.004009786 |
| Cystatin-M | Q15828 | 1474 | 1471127 | −0.115406306 | 0.000321801 | 0.011081754 |
| Dickkopf-related protein 3 | Q9UBP4 | 27122 | 360771 | −0.115572631 | 3.18599E−05 | 0.002113296 |
| Neural cell adhesion molecule 2 | O15394 | 4685 | 650716 | −0.11569215 | 0.000470212 | 0.014610556 |
| Polypeptide N- | Q8N4A0 | 100528030 | 2172221 | −0.115992029 | 0.000275245 | 0.01000261 |
| acetylgalactosaminyltransferase 4 | | | | | | |
| Ephrin type-A receptor 4 | P54764 | 2043 | 719050 | −0.116017728 | 4.77329E−06 | 0.000486279 |
| Vesicular, overexpressed in cancer, | Q96AW1 | 81552 | 1461826 | −0.116736598 | 0.000730164 | 0.019984144 |
| prosurvival protein 1 | | | | | | |
| Dual specificity protein phosphatase 13 | Q6B811 | 51207 | 652517 | −0.118423249 | 0.00168424 | 0.03489802 |
| isoform A | | | | | | |
| Ectonucleotide | Q9UJA9 | 59084 | 65565 | −0.119816018 | 0.001310567 | 0.030517487 |
| pyrophosphatase/phosphodiesterase | | | | | | |
| family member 5 | | | | | | |
| NT-3 growth factor receptor | Q16288 | 4916 | 265827 | −0.120454134 | 1.67304E−05 | 0.001319545 |
| Endothelial cell-selective adhesion | Q96AP7 | 90952 | 784184 | −0.120531638 | 2.00917E−06 | 0.000263668 |
| molecule | | | | | | |
| Dipeptidase 1 | P16444 | 1800 | 879413 | −0.120892192 | 0.000557496 | 0.016622896 |
| 3-mercaptopyruvate sulfurtransferase | P25325 | 4357 | 1268615 | −0.121577417 | 0.000117645 | 0.005427189 |
| Trafficking protein particle complex | Q86SZ2 | 122553 | 228124 | −0.121706257 | 0.000365917 | 0.012199998 |
| subunit 6B | | | | | | |
| Cell adhesion molecule 2 | Q8N3J6 | 253559 | 169073 | −0.122464599 | 1.9064E−05 | 0.001426884 |
| Anthrax toxin receptor 1 | Q9H6X2 | 84168 | 104646 | −0.122716071 | 0.000389412 | 0.012877161 |
| Dickkopf-related protein 4 | Q9UBT3 | 27121 | 2295932 | −0.125258837 | 0.001349095 | 0.030790262 |
| Gamma-glutamyltransferase 5 | P36269 | 2687 | 2154820 | −0.125980204 | 5.46632E−07 | 0.000106063 |
| Seizure 6-like protein | Q9BYH1 | 23544 | 195633 | −0.126098463 | 2.60777E−07 | 5.9775E−05 |
| EMILIN-3:region 1 | Q9NT22 | 90187 | 8773172 | −0.126267788 | 0.000440561 | 0.013971484 |
| Klotho | Q9UEF7 | 9365 | 1538415 | −0.127052504 | 0.002299912 | 0.04347901 |
| Protocadherin-10:Extracellular domain | Q9P2E7 | 57575 | 901838 | −0.127101062 | 0.000133506 | 0.006007289 |
| Transmembrane protein 132A | Q24JP5 | 54972 | 787116 | −0.128708696 | 0.000150531 | 0.006382339 |
| Syntaxin-1A | Q16623 | 6804 | 1955314 | −0.130457877 | 3.97491E−05 | 0.002470847 |
| WAP, Kazal, immunoglobulin, Kunitz and | Q8TEU8 | 124857 | 1340823 | −0.131160849 | 8.94843E−06 | 0.000790804 |
| NTR domain-containing protein 2 | | | | | | |
| Interleukin-2 receptor subunit beta | P14784 | 3560 | 934316 | −0.135614287 | 0.001303927 | 0.030459578 |
| Acetylcholinesterase | P22303 | 43 | 1098011 | −0.135853369 | 0.001518297 | 0.033072747 |
| Contactin-2 | Q02246 | 6900 | 329692 | −0.136226706 | 0.00027201 | 0.01000261 |
| Coiled-coil domain-containing protein | Q96EE4 | 90693 | 638821 | −0.137441845 | 5.29804E−05 | 0.003104813 |
| 126 | | | | | | |
| Alpha-amylase 2B | P19961 | 280 | 1555649 | −0.137738263 | 0.002002072 | 0.040123485 |
| Integrin alpha V beta 3 | P06756 \| | 3685 \| | 2018710 | −0.138175871 | 9.09188E−05 | 0.004536662 |
| | P05106 | 3690 | | | | |
| WAP, Kazal, immunoglobulin, Kunitz and | Q8TEU8 | 124857 | 323550 | −0.138800223 | 2.26272E−05 | 0.001676466 |
| NTR domain-containing protein 2 | | | | | | |
| Interleukin-1 Receptor accessory protein | Q9NPH3 | 3556 | 263012 | −0.138917587 | 0.000448871 | 0.01413078 |
| Glyoxylate reductase/hydroxypyruvate | Q9UBQ7 | 9380 | 18295102 | −0.1391542 | 0.001381898 | 0.030997625 |
| reductase | | | | | | |
| Neural cell adhesion molecule 1, 120 kDa | P13591 | 4684 | 449862 | −0.139253357 | 8.43779E−08 | 2.92504E−05 |
| isoform | | | | | | |
| Secretogranin-3 | Q8WXD2 | 29106 | 79572 | −0.140332264 | 6.27346E−06 | 0.000613544 |
| Neural cell adhesion molecule 1 | P13591 | 4684 | 2016141 | −0.140969634 | 7.72488E−09 | 5.6662E−06 |
| Netrin receptor UNC5D | Q6UXZ4 | 137970 | 514056 | −0.140970311 | 8.09148E−06 | 0.000723792 |
| Alpha-amylase 2B | P19961 | 280 | 1043957 | −0.141017983 | 0.000814517 | 0.021490936 |
| Alpha-1,3-mannosyltransferase ALG2 | Q9H553 | 85365 | 236546 | −0.141098817 | 7.11382E−05 | 0.003687169 |
| Matrix-remodeling-associated protein | Q9BRK3 | 54587 | 1052110 | −0.141883006 | 6.07316E−07 | 0.000106063 |
| 8:Extracellular domain | | | | | | |
| Receptor-type tyrosine-protein | P23468 | 5789 | 929615 | −0.142576901 | 7.85543E−06 | 0.00071662 |
| phosphatase delta | | | | | | |
| Interleukin-1 Receptor accessory protein | Q9NPH3 | 3556 | 140487 | −0.143862699 | 0.000512169 | 0.015523811 |
| A disintegrin and metalloproteinase with | Q76LX8 | 11093 | 317551 | −0.144358392 | 7.59864E−06 | 0.000710306 |
| thrombospondin motifs 13 | | | | | | |
| Dickkopf-related protein 3 | Q9UBP4 | 27122 | 1074624 | −0.145551734 | 1.82455E−05 | 0.001379697 |
| Gamma-enolase | P09104 | 2026 | 1033948 | −0.147011772 | 0.000670345 | 0.018766665 |
| Glypican-3 | P51654 | 2719 | 484262 | −0.152232669 | 0.00066956 | 0.018766665 |
| Kynurenine -- oxoglutarate transaminase 3 | Q6YP21 | 56267 | 126825 | −0.15585502 | 0.002717429 | 0.048734338 |
| Calcium and integrin-binding protein 1 | Q99828 | 10519 | 2042340 | −0.157349946 | 0.000115335 | 0.005354311 |
| Prostaglandin F2 receptor negative | Q9P2B2 | 5738 | 127277 | −0.157857878 | 1.856E−06 | 0.000256864 |
| regulator | | | | | | |
| SLIT and NTRK-like protein 5 | O94991 | 26050 | 456817 | −0.159922602 | 3.62324E−06 | 0.000402673 |
| Activating signal cointegrator 1 complex | Q9H118 | 84164 | 2447618 | −0.160045081 | 3.18411E−05 | 0.002113296 |
| subunit 2 | | | | | | |
| DNA-directed RNA polymerases I, II, and | P53803 | 5440 | 1913466 | −0.163330741 | 0.00075276 | 0.020374507 |
| III subunit RPABC4 | | | | | | |
| Brevican core protein | Q96GW7 | 63827 | 346158 | −0.163558495 | 6.22344E−08 | 2.43716E−05 |
| Peroxisomal carnitine O- | Q9UKG9 | 54677 | 1392927 | −0.164031115 | 0.00161388 | 0.034412242 |
| octanoyltransferase | | | | | | |
| Neuronal pentraxin receptor | O95502 | 23467 | 89974 | −0.165053517 | 4.70561E−06 | 0.000486136 |
| Ciliary neurotrophic factor receptor | P26992 | 1271 | 27116 | −0.165168937 | 1.92086E−07 | 4.70609E−05 |
| subunit alpha | | | | | | |
| Netrin receptor UNC5D | Q6UXZ4 | 137970 | 1630722 | −0.16568089 | 2.19646E−06 | 0.000280525 |
| Biglycan | P21810 | 633 | 1369026 | −0.166288149 | 0.000173044 | 0.007026729 |
| Adhesion G protein-coupled receptor F5 | Q8IZF2 | 221395 | 640957 | −0.168238551 | 0.002745988 | 0.049126394 |
| SLIT and NTRK-like protein 1 | Q96PX8 | 114798 | 1553915 | −0.168977179 | 2.64957E−05 | 0.001905349 |
| Serine-rich single-pass membrane protein | Q8WWF3 | 136263 | 82988 | −0.174744419 | 0.002396018 | 0.044681682 |
| 1 | | | | | | |
| IgLON family member 5 | A6NGN9 | 402665 | 64782 | −0.17524826 | 6.31302E−08 | 2.43716E−05 |
| Voltage-dependent calcium channel | Q8IZS8 | 55799 | 88856 | −0.177303038 | 2.07673E−07 | 4.91381E−05 |
| subunit alpha-2/delta-3 | | | | | | |
| Pancreatic alpha-amylase | P04746 | 279 | 1891753 | −0.177697499 | 6.43898E−05 | 0.003498515 |
| Ephrin type-A receptor 6 | Q9UF33 | 285220 | 2258737 | −0.178208518 | 3.25122E−07 | 7.01402E−05 |
| Interleukin-35 | P29459 \| | 3592 \| | 2053339 | −0.180215238 | 0.000996967 | 0.024957387 |
| | Q14213 | 10148 | | | | |
| Integrin a11b1 | Q9UKX5 \| | 22801 \| | 2169811 | −0.180714605 | 2.8278E−05 | 0.001995788 |
| | P05556 | 3688 | | | | |
| Adiponectin | Q15848 | 9370 | 355424 | −0.183859822 | 0.001191052 | 0.028379268 |
| Fc receptor-like protein 4: Extracellular | Q96PJ5 | 83417 | 897323 | −0.184013599 | 0.001448491 | 0.032002054 |
| domain | | | | | | |
| Histone-lysine N-methyltransferase | Q96KQ7 | 10919 | 584360 | −0.185602597 | 2.90714E−05 | 0.001995788 |
| EHMT2 | | | | | | |
| THAP domain-containing protein 4 | Q8WY91 | 51078 | 2449016 | −0.186142195 | 4.52862E−06 | 0.000474535 |
| Interleukin-27 subunit beta | Q14213 | 10148 | 1085177 | −0.186819989 | 0.001088877 | 0.02671209 |
| Myelin-associated glycoprotein | P20916 | 4099 | 2057950 | −0.189241569 | 1.95106E−09 | 2.86221E−06 |
| Pancreatic triacyl glycerol lipase | P16233 | 5406 | 1561316 | −0.189401343 | 0.001394829 | 0.031125362 |
| Cerebellin-2 | Q8IUK8 | 147381 | 218872 | −0.191378213 | 4.32014E−07 | 9.05378E−05 |
| Cysteine-rich with EGF-like domain | Q96HD1 | 78987 | 762840 | −0.191752477 | 0.000198185 | 0.007691479 |
| protein 1 | | | | | | |
| Ecto-ADP-ribosyltransferase 3 | Q13508 | 419 | 7970315 | −0.193928751 | 1.76026E−07 | 4.70609E−05 |
| Ecto-ADP-ribosyltransferase 3 | Q13508 | 419 | 109703 | −0.194660819 | 3.44859E−08 | 1.92331E−05 |
| AP-1 complex-associated regulatory | Q63HQ0 | 55435 | 2044529 | −0.199120577 | 0.000431223 | 0.013812308 |
| protein | | | | | | |
| Neuronal pentraxin receptor | O95502 | 23467 | 1551137 | −0.199526266 | 5.96642E−07 | 0.000106063 |
| Neurocan core protein | O14594 | 1463 | 15573110 | −0.207735202 | 5.18679E−07 | 0.000105681 |
| Cerebellin-1 | P23435 | 869 | 931327 | −0.213222283 | 6.40396E−05 | 0.003498515 |
| Cystatin-SA | P09228 | 1470 | 432433 | −0.216126995 | 0.000684126 | 0.018864899 |
| ADP-ribosylation factor 4 | P18085 | 378 | 1840826 | −0.220782131 | 0.002169365 | 0.042207671 |
| DCC | P43146 | 1630 | 2168529 | −0.220917912 | 1.97149E−06 | 0.000263668 |
| Amyloid-like protein 1 | P51693 | 333 | 721025 | −0.242015658 | 1.02059E−06 | 0.000162739 |
| Pyruvate dehydrogenase E1 component | P29803 | 5161 | 2524933 | −0.257010161 | 0.001884835 | 0.037992767 |
| subunit alpha, testis-specific form, | | | | | | |
| mitochondrial | | | | | | |
| Advanced glycosylation end product- | Q15109 | 177 | 412552 | −0.258075311 | 0.002438423 | 0.045280593 |
| specific receptor, soluble | | | | | | |
| Galactosylgalactosylxylosylprotein 3-beta- | Q9P2W7 | 27087 | 2177018 | −0.267319554 | 0.000105759 | 0.004941018 |
| glucuronosyltransferase 1 | | | | | | |
| Oligodendrocyte-myelin glycoprotein | P23515 | 4974 | 169085 | −0.269338076 | 4.19535E−08 | 1.92331E−05 |
| Lactase-phlorizin hydrolase | P09848 | 3938 | 901758 | −0.285236705 | 0.001373375 | 0.030900943 |
| Insulin-like growth factor-binding protein | P08833 | 3484 | 277135 | −0.343649537 | 0.000551362 | 0.016574754 |
| 1 | | | | | | |
| SLIT and NTRK-like protein 3 | O94933 | 22865 | 1056519 | −0.346064338 | 1.87193E−07 | 4.70609E−05 |
| Carbonic anhydrase 6 | P23280 | 765 | 137479 | −0.353980918 | 5.86883E−09 | 4.7831E−06 |
| Carbonic anhydrase 6 | P23280 | 765 | 335280 | −0.392137071 | 3.90422E−08 | 1.92331E−05 |

TABLE 5
Protein aptamer associations with accelerated decline FEV1 trajectory
(vs normal decline trajectory) in multivariable regression analyses.

| | | Entrez | | Log | Lower | Upper | | |
|---|---|---|---|---|---|---|---|---|
| Target Protein | UniProt | Gene | Aptamer | Fold | 95% | 95% | | FDR |
| Name | ID | ID | ID | Change | CI | CI | p-value | p-value |
| Nuclear | Q9GZM8 | 81565 | 235456 | 0.249 | 0.129 | 0.368 | 4.65E−05 | 1.89E−02 |
| distribution | | | | | | | | |
| protein nudE-like 1 | | | | | | | | |
| Ribonuclease | P07998 | 6035 | 72112 | 0.207 | 0.095 | 0.319 | 2.99E−04 | 4.77E−02 |
| pancreatic | | | | | | | | |
| Estradiol 17- | P14061 | 3292 | 47083 | 0.197 | 0.123 | 0.271 | 1.78E−07 | 1.06E−03 |
| beta- | | | | | | | | |
| dehydrogenase 1 | | | | | | | | |
| Phospholipase | P14555 | 5320 | 269274 | 0.186 | 0.092 | 0.280 | 1.06E−04 | 2.88E−02 |
| A2, membrane | | | | | | | | |
| associated | | | | | | | | |
| Protein S100-A9 | P06702 | 6280 | 533949 | 0.156 | 0.075 | 0.238 | 1.68E−04 | 3.93E−02 |
| Triggering | Q9NP99 | 54210 | 92661 | 0.149 | 0.071 | 0.227 | 1.79E−04 | 3.93E−02 |
| receptor | | | | | | | | |
| expressed on | | | | | | | | |
| myeloid cells 1 | | | | | | | | |
| Ribonuclease K6 | Q93091 | 6039 | 564620 | 0.140 | 0.069 | 0.212 | 1.27E−04 | 3.20E−02 |
| Cystatin B | P04080 | 1476 | 1976813 | 0.128 | 0.060 | 0.195 | 2.09E−04 | 3.93E−02 |
| Osteopetrosis- | Q86WC4 | 28962 | 1935325 | 0.125 | 0.065 | 0.185 | 4.28E−05 | 1.89E−02 |
| associated | | | | | | | | |
| transmembrane | | | | | | | | |
| protein 1 | | | | | | | | |
| Delta-like protein 1 | O00548 | 28514 | 826443 | 0.123 | 0.062 | 0.184 | 8.54E−05 | 2.85E−02 |
| Retinoic acid | Q99969 | 5919 | 307962 | 0.122 | 0.058 | 0.186 | 2.06E−04 | 3.93E−02 |
| receptor | | | | | | | | |
| responder | | | | | | | | |
| protein 2 | | | | | | | | |
| 5- | P50406 | 3362 | 135615 | 0.120 | 0.063 | 0.177 | 4.37E−05 | 1.89E−02 |
| hydroxytryptamine | | | | | | | | |
| receptor 6 | | | | | | | | |
| 2-methoxy-6- | Q5HYK3 | 84274 | 2211230 | 0.111 | 0.053 | 0.170 | 1.99E−04 | 3.93E−02 |
| polyprenyl-1,4- | | | | | | | | |
| benzoquinol | | | | | | | | |
| methylase, | | | | | | | | |
| mitochondrial | | | | | | | | |
| Collagen alpha- | Q2UY09 | 340267 | 107021 | 0.110 | 0.058 | 0.162 | 3.71E−05 | 1.89E−02 |
| 1(XXVIII) chain | | | | | | | | |
| Collagen alpha- | P12111 | 1293 | 1119631 | 0.109 | 0.059 | 0.159 | 2.07E−05 | 1.65E−02 |
| 3(VI) | | | | | | | | |
| chain:Bovine | | | | | | | | |
| pancreatic | | | | | | | | |
| trypsin | | | | | | | | |
| inhibitor/Kunitz | | | | | | | | |
| inhibitor domain, | | | | | | | | |
| isoform 1 | | | | | | | | |
| Corticotropin- | P24387 | 1393 | 603924 | 0.104 | 0.048 | 0.161 | 2.76E−04 | 4.72E−02 |
| releasing factor- | | | | | | | | |
| binding protein | | | | | | | | |
| Complement C1r | Q9NZP8 | 51279 | 93481 | 0.099 | 0.047 | 0.151 | 1.89E−04 | 3.93E−02 |
| subcomponent- | | | | | | | | |
| like protein | | | | | | | | |
| Gamma- | O95166 | 11337 | 17735130 | 0.099 | 0.052 | 0.146 | 3.92E−05 | 1.89E−02 |
| aminobutyric | | | | | | | | |
| acid receptor- | | | | | | | | |
| associated | | | | | | | | |
| protein | | | | | | | | |
| Gamma- | Q9H0R8 | 23710 | 1266144 | 0.098 | 0.046 | 0.149 | 1.95E−04 | 3.93E−02 |
| aminobutyric | | | | | | | | |
| acid receptor- | | | | | | | | |
| associated | | | | | | | | |
| protein-like 1 | | | | | | | | |
| Serine/threonine- | O94768 | 9262 | 83996 | 0.095 | 0.048 | 0.143 | 8.30E−05 | 2.85E−02 |
| protein kinase 17B | | | | | | | | |
| Leukemia | P42702 | 3977 | 583749 | −0.090 | −0.137 | −0.044 | 1.53E−04 | 3.74E−02 |
| inhibitory factor | | | | | | | | |
| receptor | | | | | | | | |
| Hedgehog- | Q96QV1 | 64399 | 1083364 | −0.094 | −0.145 | −0.043 | 2.91E−04 | 4.74E−02 |
| interacting | | | | | | | | |
| protein | | | | | | | | |
| Semaphorin-3G | Q9NS98 | 56920 | 562821 | −0.096 | −0.144 | −0.048 | 1.01E−04 | 2.86E−02 |
| Protein | Q8N128 | 283635 | 803941 | −0.105 | −0.161 | −0.050 | 2.23E−04 | 3.99E−02 |
| FAM177A1 | | | | | | | | |
| Neural cell | P13591 | 4684 | 2016141 | −0.108 | −0.159 | −0.056 | 4.05E−05 | 1.89E−02 |
| adhesion | | | | | | | | |
| molecule 1 | | | | | | | | |
| SLIT and NTRK- | Q8IW52 | 139065 | 713914 | −0.111 | −0.172 | −0.051 | 3.16E−04 | 4.87E−02 |
| like protein 4 | | | | | | | | |
| Tubulointerstitial | Q9GZM7 | 64129 | 11192168 | −0.112 | −0.172 | −0.051 | 2.86E−04 | 4.74E−02 |
| nephritis | | | | | | | | |
| antigen-like | | | | | | | | |
| NT-3 growth | Q16288 | 4916 | 265827 | −0.112 | −0.169 | −0.055 | 1.15E−04 | 3.02E−02 |
| factor receptor | | | | | | | | |
| Uronyl 2- | Q9Y2C2 | 10090 | 836474 | −0.112 | −0.168 | −0.056 | 9.81E−05 | 2.86E−02 |
| sulfotransferase | | | | | | | | |
| Protocadherin-9 | Q9HC56 | 5101 | 1055826 | −0.118 | −0.180 | −0.055 | 2.49E−04 | 4.35E−02 |
| Seizure 6-like | Q9BYH1 | 23544 | 195633 | −0.126 | −0.177 | −0.076 | 1.20E−06 | 1.46E−03 |
| protein | | | | | | | | |
| Neurogenic locus | Q04721 | 4853 | 840784 | −0.127 | −0.176 | −0.078 | 4.32E−07 | 1.06E−03 |
| notch homolog | | | | | | | | |
| protein 2 | | | | | | | | |
| WAP, Kazal, | Q8TEU8 | 124857 | 1340823 | −0.128 | −0.190 | −0.067 | 5.02E−05 | 1.94E−02 |
| immunoglobulin, | | | | | | | | |
| Kunitz and NTR | | | | | | | | |
| domain- | | | | | | | | |
| containing | | | | | | | | |
| protein 2 | | | | | | | | |
| Dickkopf-related | Q9UBP4 | 27122 | 1074624 | −0.131 | −0.202 | −0.060 | 3.19E−04 | 4.87E−02 |
| protein 3 | | | | | | | | |
| IgLON family | A6NGN9 | 402665 | 64782 | −0.132 | −0.198 | −0.066 | 9.14E−05 | 2.86E−02 |
| member 5 | | | | | | | | |
| Histatin-3 | P15516 | 3347 | 106031 | −0.142 | −0.217 | −0.067 | 2.01E−04 | 3.93E−02 |
| WAP, Kazal, | Q8TEU8 | 124857 | 323550 | −0.144 | −0.213 | −0.075 | 4.27E−05 | 1.89E−02 |
| immunoglobulin, | | | | | | | | |
| Kunitz and NTR | | | | | | | | |
| domain- | | | | | | | | |
| containing | | | | | | | | |
| protein 2 | | | | | | | | |
| Brevican core | Q96GW7 | 63827 | 346158 | −0.155 | −0.219 | −0.090 | 3.02E−06 | 3.16E−03 |
| protein | | | | | | | | |
| Cerebellin-2 | Q8IUK8 | 147381 | 218872 | −0.158 | −0.238 | −0.079 | 9.85E−05 | 2.86E−02 |
| Myelin- | P20916 | 4099 | 2057950 | −0.162 | −0.227 | −0.097 | 1.14E−06 | 1.46E−03 |
| associated | | | | | | | | |
| glycoprotein | | | | | | | | |
| Alpha-amylase | P19961 | 280 | 1555649 | −0.182 | −0.279 | −0.086 | 2.18E−04 | 3.99E−02 |
| 2B | | | | | | | | |
| Neurocan core | O14594 | 1463 | 15573110 | −0.210 | −0.294 | −0.127 | 8.87E−07 | 1.46E−03 |
| protein | | | | | | | | |
| Mannan-binding | P48740 | 5648 | 809116 | −0.215 | −0.320 | −0.109 | 6.69E−05 | 2.45E−02 |
| lectin serine | | | | | | | | |
| protease | | | | | | | | |
| 1:Mannan- | | | | | | | | |
| binding lectin | | | | | | | | |
| serine protease 1 | | | | | | | | |
| heavy chain | | | | | | | | |
| Oligodendrocyte- | P23515 | 4974 | 169085 | −0.234 | −0.337 | −0.131 | 9.37E−06 | 8.59E−03 |
| myelin | | | | | | | | |
| glycoprotein | | | | | | | | |
| Cystatin-D | P28325 | 1473 | 380310 | −0.248 | −0.378 | −0.118 | 1.97E−04 | 3.93E−02 |
| Amyloid-like | P51693 | 333 | 721025 | −0.259 | −0.359 | −0.159 | 4.12E−07 | 1.06E−03 |
| protein 1 | | | | | | | | |
| Carbonic | P23280 | 765 | 137479 | −0.261 | −0.385 | −0.138 | 3.53E−05 | 1.89E−02 |
| anhydrase 6 | | | | | | | | |
| Carbonic | P23280 | 765 | 335280 | −0.322 | −0.471 | −0.174 | 2.25E−05 | 1.65E−02 |
| anhydrase 6 | | | | | | | | |

### Proteomic Risk Score of Increased Respiratory Susceptibility

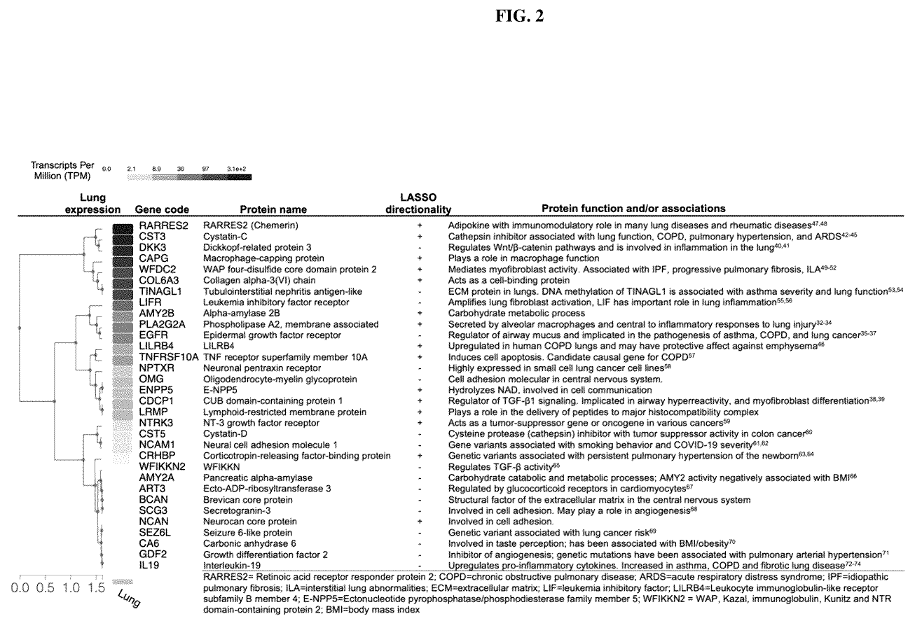

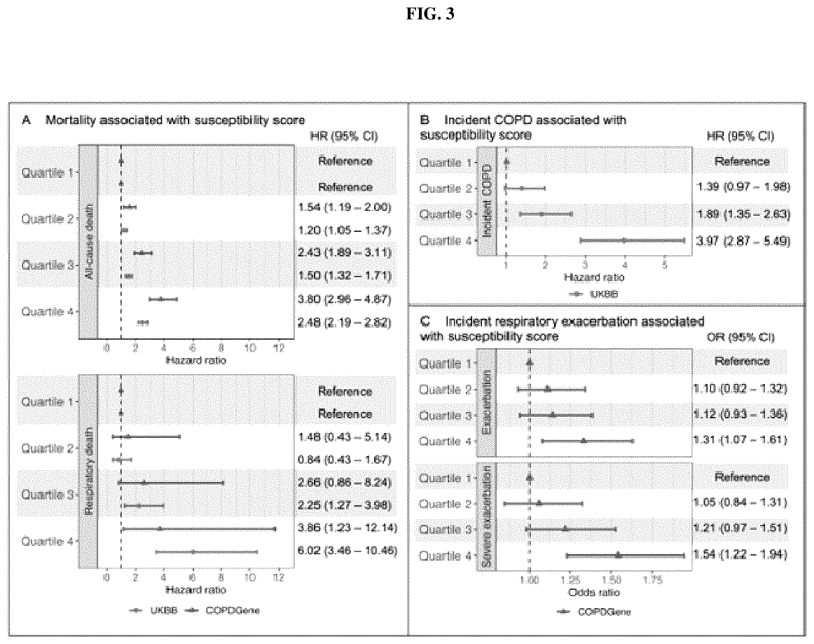

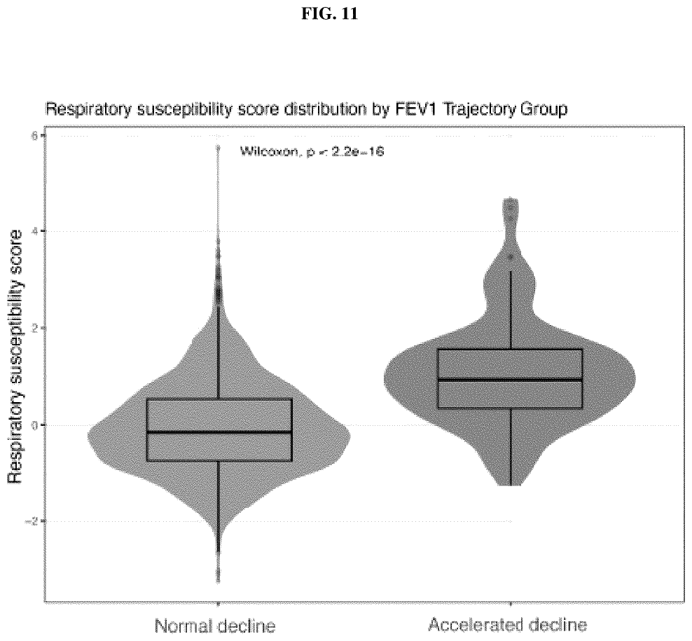

Susceptibility score was derived in the CARDIA cohort by LASSO regression and was composed of 32 proteins with their LASSO coefficients listed in Table 6. FIG. 2 displays the proteins in the susceptibility score along with their estimated lung-specific gene expression from the GTEx Portal, listing each protein's function and/or associations with lung health and disease. The mean susceptibility score (calculated as a z-score) was 1.05 in the AD group and −0.06 in the ND group (difference=1.11, 95% CI 0.95-1.28), with distributions of susceptibility score depicted in FIG. 11. In multivariable analyses stratified by smoking status at time of proteomics measurement, it was found that the susceptibility score was associated with 25-year decline in FEV1 percent predicted regardless of smoking status, including among never smokers, and with the largest association among current smokers (Table 9).

TABLE 9
Causes of respiratory death in the UK Biobank cohort, all cases.

| Cause of death | Count | Percentage |
|---|---|---|
| Abscess of lung with pneumonia | 1 | 0.3% |
| Acute bronchitis, unspecified | 1 | 0.3% |
| Alveolar and parietoalveolar conditions | 1 | 0.3% |
| Asthma, unspecified | 7 | 2.0% |
| Bronchiectasis | 10 | 2.9% |
| Bronchitis, not specified as acute or chronic | 1 | 0.3% |
| Bronchopneumonia, unspecified | 15 | 4.4% |
| Chronic obstructive pulmonary disease with acute exacerbation, unspecified | 16 | 4.7% |
| Chronic obstructive pulmonary disease with acute lower respiratory infection | 57 | 16.7% |
| Chronic obstructive pulmonary disease, unspecified | 52 | 15.2% |
| Chronic obstructive pulmonary disease with acute lower | 1 | 0.3% |
| respiratory infection/chronic obstructive pulmonary | | |
| disease, unspecified | | |
| Emphysema, unspecified | 7 | 2.0% |
| Hypersensitivity pneumonitis due to unspecified organic dust | 1 | 0.3% |
| Hypostatic pneumonia, unspecified | 1 | 0.3% |
| Influenza due to certain identified influenza virus | 1 | 0.3% |
| Influenza with other manifestations, virus not identified | 1 | 0.3% |
| Influenza with other respiratory manifestations, influenza virus identified | 4 | 1.2% |
| Influenza with other respiratory manifestations, virus not identified | 2 | 0.6% |
| Influenza with pneumonia, influenza virus identified | 2 | 0.6% |
| Influenza with pneumonia, virus not identified | 2 | 0.6% |
| Interstitial pulmonary disease, unspecified | 14 | 4.1% |
| Lobar pneumonia, unspecified | 2 | 0.6% |
| Other disorders of lung | 3 | 0.9% |
| Other interstitial pulmonary diseases with fibrosis | 82 | 24.0% |
| Other specified respiratory disorders | 4 | 1.2% |
| Pneumoconiosis due to asbestos and other mineral fibres | 1 | 0.3% |
| Pneumonia due to Pseudomonas | 1 | 0.3% |
| Pneumonia, unspecified | 38 | 11.1% |
| Pneumonitis due to food and vomit | 6 | 1.8% |
| Unspecified acute lower respiratory infection | 8 | 2.3% |
| Total respiratory death | 342 | 100.0% |

TABLE 10
Causes of respiratory death in the COPDGene cohort, all cases.

| Cause of death | Count | Percentage |
|---|---|---|
| COPD exacerbation with pneumonia | 8 | 18.2% |
| COPD exacerbation without pneumonia | 7 | 15.9% |
| COPD without exacerbation | 24 | 54.5% |
| Respiratory other | 5 | 11.4% |
| Total respiratory death | 44 | 100.0% |

TABLE 6
Coefficients for Proteomic Respiratory Susceptibility Score.

| | Entrez | |
|---|---|---|
| Protein name | gene ID | Coefficient |
| Intercept | | 0.012 |
| Phospho [symbol] e A2, membrane associated, Group [symbol] A | 5320 | 0.294 |
| CU [symbol] domain-containing protein 1 | 64866 | 0.268 |
| Collagen alpha-3(VI) chain:Bovine pancreatic | 1293 | 0.235 |
| trypsin | | |
| inhibitor/Kun [symbol] inhibitor domain, isoform 1 | | |
| Macrophage-capping protein | 822 | 0.201 |
| WAP four-disu [symbol] de core domain protein 2 | 1040 [symbol] | 0.177 |
| Lymphoid-restricted membrane protein | 4033 | 0.168 |
| Leukocyte im [symbol] e receptor subfamily B member 4 | 1100 [symbol] | 0.165 |
| Cystatin-C | 1471 | 0.14 |
| NT-3 growth factor receptor | 491 [symbol] | 0.14 |
| Co [symbol] otropin-releasing factor-bindi [symbol] protein | 1393 | 0.134 |
| Tumor necrosis factor receptor superfamily | [symbol] 797 | 0.121 |
| member 10A | | |
| Neurocan core protein | 14 [symbol] | 0.0 [symbol] 6 |
| Alpha-a [symbol] lase 2B | 280 | 0.054 |
| E [symbol] eotide pyrophosph [symbol] /phosp [symbol] se family | [symbol] 084 | 0.0 [symbol] |
| member [symbol] | | |
| [symbol] oic acid receptor responder protein 2 | 5 [symbol] 1 [symbol] | 0.0 [symbol] 1 |
| (Chemerin) | | |
| Tubulointerstitial nephritis antigen-like | 64129 | −0.012 |
| Secretog [symbol] nin- [symbol] | 29106 | −0.016 |
| Brevican core protein | 63827 | −0.027 |
| WAP, Kazal, immunoglobulin, Kunitz and NTR | 124857 | −0.036 |
| domain-containing protein 2 | | |
| Epidermal growth factor receptor | 1956 | −0.039 |
| Leukemia inhibitory factor receptor | 3977 | −0.042 |
| Neuronal pentraxin receptor | 234 [symbol] 7 | −0.056 |
| Pancreatic alpha-a [symbol] lase | 279 | −0.074 |
| Neural cell adhesion molecule 1, 120 kDa isoform | 4 [symbol] 84 | −0.093 |
| Cystatin-D | 1473 | −0.103 |
| Ecto-ADP-ribos [symbol] e 3 | 419 | −0.145 |
| Seizure [symbol] e protein | 23544 | −0.147 |
| Carbonic anhydrase 6 | 765 | −0.152 |
| Growth differentiation factor 2 (BM [symbol] ) | 2658 | −0.154 |
| Dic [symbol] -related protein 3 | 27122 | −0.16 |
| interleukin-19 | 29949 | −0.161 |
| Oligodendrocyte-myelin glycoprotein | 4974 | −0.23 |

[symbol]

ceptibility score calculated for each individual as the sum of the intercept plus the scaled log2-transformed protein values multiplied by the corresponding coefficients. The susceptibility score was derived in the CARDIA cohort. [symbol]
indicates data missing or illegible when filed

TABLE 7
Association between susceptibility score (measured at Year
25) and yearly change in FEV1 percent predicted from Year
5 to Year 30, by Year 25 smoking status in the CARDIA cohort.

| | Susceptibility | Lower | Upper | |
|---|---|---|---|---|
| Group | score estimate* | 95% CI | 95% CI | p-value |
| Never smokers | −0.12% | −0.14% | −0.10% | 2E−16 |
| Former smokers | −0.15% | −0.18% | −0.12% | 2E−16 |
| Current smokers | −0.18% | −0.23% | −0.14% | 2E−15 |
| All participants | −0.15% | −0.16% | −0.13% | 2E−16 |

All analyses adjusted for age, sex, race, center, Y 25 BMI. Additionally adjusted for Y 25 smoking pack-years in analyses of all participants, former smokers, and current smokers.
*Estimate is per 1 standard deviation difference in susceptibilty score

Association of Susceptibility Score with Future Lung Function Decline

The UKBB and COPDGene cohort characteristics by quartile of susceptibility score are presented in Table 2. It was first examined whether the susceptibility score was associated with future lung function decline over 5 years from time of proteomics measurement in COPDGene. It was found that participants with the highest quartile of susceptibility score had decline in post-bronchodilator FEV1 that was 9.5 ml/year (95% CI 0.25-19.0) greater than those in the lowest quartile (Table 8). There was no statistically significant difference in FEV1 decline between quartile 1 and 2 or 3.

TABLE 2
Characteristics of UK Biobank and COPDGene cohorts by quartile of proteomic respiratory susceptibility score.

| | UK Biobank | | | | | | | | COPDGene | | | | | | | |
|---|---|---|---|---|---|---|---|---|---|---|---|---|---|---|---|---|
| | Quartile 1, | | Quartile 2, | | Quartile 3, | | Quartile 4, | | Quartile 1, | | Quartile 2, | | Quartile 3, | | Quartile 4, | |
| Characteristic | N = 8,750 | | N = 8,750 | | N = 8,750 | | N = 8,750 | | N = 1,298 | | N = 1,299 | | N = 1,291 | | N = 1,280 | |
| Susceptibility | −1.40 | (0.47) | −0.44 | (0.20) | 0.29 | (0.23) | 1.54 | (0.72) | −1.29 | (0.46) | −0.39 | (0.18) | 0.28 | (0.21) | 1.39 | (0.70) |
| score* | | | | | | | | | | | | | | | | |
| Age* | 53 | (8) | 56 | (8) | 58 | (8) | 60 | (7) | 64 | (8) | 66 | (9) | 66 | (9) | 66 | (9) |
| Female, n (%) | 4,891 | (56) | 4,719 | (54) | 4,618 | (53) | 4,639 | (53) | 655 | (50) | 625 | (48) | 643 | (50) | 635 | (50) |
| Race, n (%) | | | | | | | | | | | | | | | | |
| Asian | 198 | (2.3) | 183 | (2.1) | 176 | (2.0) | 173 | (2.0) | 0 | | 0 | | 0 | | 0 | |
| Black | 324 | (3.7) | 159 | (1.8) | 155 | (1.8) | 114 | (1.3) | 342 | (26) | 336 | (26) | 377 | (29) | 448 | (35) |
| Mixed | 89 | (1.0) | 54 | (0.6) | 47 | (0.5) | 48 | (0.5) | 0 | | 0 | | 0 | | 0 | |
| Unknown/other | 186 | (2.1) | 148 | (1.7) | 121 | (1.4) | 114 | (1.3) | 0 | | 0 | | 0 | | 0 | |
| White | 7,953 | (91) | 8,206 | (94) | 8,251 | (94) | 8,301 | (95) | 956 | (74) | 963 | (74) | 914 | (71) | 832 | (65) |
| BMI* | 24.9 | (3.3) | 26.6 | (3.7) | 28.1 | (4.3) | 30.3 | (5.6) | 26.4 | (4.7) | 28.4 | (5.4) | 29.8 | (6.3) | 31.5 | (7.6) |
| Smoking | | | | | | | | | | | | | | | | |
| status, n (%) | | | | | | | | | | | | | | | | |
| Current | 400 | (4.6) | 606 | (6.9) | 924 | (11) | 1,731 | (20) | 338 | (26) | 469 | (36) | 542 | (42) | 634 | (50) |
| Former | 2,590 | (30) | 2,984 | (34) | 3,302 | (38) | 3,308 | (38) | 960 | (74) | 830 | (64) | 749 | (58) | 646 | (50) |
| Never | 5,754 | (66) | 5,148 | (59) | 4,513 | (52) | 3,700 | (42) | — | | — | | — | | — | |
| Missing | 6 | (<1) | 12 | (<1) | 11 | (<1) | 11 | (<1) | — | | — | | — | | — | |
| Pack-years* | 3 | (8) | 5 | (12) | 8 | (15) | 13 | (21) | 37 | (21) | 44 | (22) | 46 | (24) | 49 | (26) |

| | | | | | | | | | | | | |
|---|---|---|---|---|---|---|---|---|---|---|---|---|
| Deprivation | −1.5 | (3.1) | −1.4 | (3.1) | −1.2 | (3.1) | −0.7 | (3.3) | — | — | — | — |
| Index*^{†} | | | | | | | | | | | | |
| Income, n (%) | | | | | | | | | | | | |

| | | | | | | | | | | | | |
|---|---|---|---|---|---|---|---|---|---|---|---|---|
| <$15K | — | — | — | — | 253 | (19) | 310 | (24) | 374 | (29) | 466 | (36) |
| $15K-35K | — | — | — | — | 278 | (21) | 298 | (23) | 315 | (24) | 297 | (23) |
| $35K-50K | — | — | — | — | 186 | (14) | 194 | (15) | 167 | (13) | 142 | (11) |
| $50K-5K | — | — | — | — | 206 | (16) | 177 | (14) | 147 | (11) | 108 | (8.4) |
| >$75K | — | — | — | — | 213 | (16) | 156 | (12) | 118 | (9.1) | 77 | (6.0) |
| Decline to | — | — | — | — | 162 | (12) | 164 | (13) | 170 | (13) | 190 | (15) |
| answer | | | | | | | | | | | | |

| | | | | | | | | | | | | |
|---|---|---|---|---|---|---|---|---|---|---|---|---|
| Respiratory | 1,041 | (12) | 1,129 | (13) | 1,194 | (14) | 1,536 | (18) | — | — | — | — |
| disease^{§}, n (%) | | | | | | | | | | | | |

| | | | | | | | | | | | | |
|---|---|---|---|---|---|---|---|---|---|---|---|---|
| FEV1/FVC | — | — | — | — | 447 | (34) | 579 | (45) | 651 | (50) | 639 | (50) |
| ratio <0.7, | | | | | | | | | | | | |
| n (%) | | | | | | | | | | | | |
| FEV1 % | — | — | — | — | 86 | (24) | 79 | (25) | 74 | (24) | 72 | (23) |
| predicted* | | | | | | | | | | | | |

TABLE 8
Association between quartile of susceptibility score
and yearly change in post-bronchodilator FEV1.

| | FEV1 change | Lower | Upper | |
|---|---|---|---|---|
| Quartile | (mL/year) | 95% CI | 95% CI | p-value |
| 1 | Ref | Ref | Ref | |
| 2 | −0.15% | −0.18% | −0.12% | 2E−16 |
| 3 | −0.18% | −0.23% | −0.14% | 2E−15 |
| 4 | −0.15% | −0.16% | −0.13% | 2E−16 |

Association of Susceptibility Score with all-Cause Mortality and Respiratory Mortality

In multivariable models, one standard deviation higher susceptibility score was significantly associated with all-cause mortality in both cohorts: 56% higher risk in UKBB (HR 1.56; 95% CI 1.50-1.61) and 75% higher in COPDGene (HR 1.75; 95% CI 1.63-1.88). Each standard deviation higher susceptibility score was significantly associated with over 2 times the risk of respiratory death in UKBB (HR, 2.39; 95% CI 2.16-2.64) and 81% higher risk in COPDGene (HR, 1.81; 95% CI 1.32-2.47). A dose-response relationship was found between susceptibility score quartile and risk of all-cause death and respiratory death (FIG. 2). Unadjusted cumulative event plots for all-cause mortality in both cohorts are displayed in FIG. 3. The number of participants, deaths, and follow-up time for each cohort are shown in Table 3. Causes of death for the two cohorts are shown in Tables 9-10.

TABLE 3
Cox model results for association with respiratory susceptibility
score from CARDIA, UK Biobank, COPDGene.

| | | | | Median | Lower | Upper | | | |
|---|---|---|---|---|---|---|---|---|---|
| | | N | Events | follow-up | IQR | IQR | | | |
| | | (complete | (complete | time | follow-up | follow-up | | | |
| Cohort | Outcome | case) | case) | (years) | time | time | Beta | SE | HR |
| UK Biobank | All-cause death | 34744 | 3714 | 13.7 | 13.0 | 14.5 | 0.44 | 0.02 | 1.56 |
| UK Biobank | Respiratory death | 34744 | 352 | 13.7 | 13.0 | 14.5 | 0.87 | 0.05 | 2.39 |
| UK Biobank | Incident COPD | 26028 | 765 | 23.4 | 12.7 | 14.1 | 0.51 | 0.04 | 1.04 |
| COPDGene | All-cause death | 5162 | 875 | 5.5 | 4.9 | 7.5 | 0.56 | 0.04 | 1.75 |
| COPDGene | Respiratory death | 4852 | 64 | 2.9 | 2 | 3.8 | 0.50 | 0.20 | 1.84 |

| | Lower | Upper | | | |
|---|---|---|---|---|---|
| | 95th | 95th | | | |
| Cohort | CI | CI | P-value | Description | Exclusions |
| UK Biobank | 1.50 | 1.61 | 1.0E−131 | N/A | none |
| UK Biobank | 1.16 | 2.64 | 1.4E−65 | Primary cause | none |
| | | | | of death listed | |
| | | | | as ICD-10 code | |
| | | | | 100-199 | |
| UK Biobank | 1.71 | 1.88 | 5.0E−59 | Phecode 498: | Baseline self-reported |
| | | | | Chronic airway | asthma, emphysema, |
| | | | | obstruction | COPD, chronic |
| | | | | | bronchitis, or |
| | | | | | respiratory failure; |
| | | | | | Doctor-diagnosed |
| | | | | | COPD, asthma, |
| | | | | | emphysema, |
| | | | | | chronic bronchitis |
| COPDGene | 1.55 | 1.85 | <2E−15 | N/A | none |
| COPDGene | 1.34 | 2.53 | 2.0E−04 | Adjudicated | none |
| | | | | as respiratory | |
| | | | | primary cause | |
| | | | | of death by | |
| | | | | COPDGene | |
| | | | | committee | |

N = number of participant;
IQR = Interquartile range;
CI = confidence interval

Association of Susceptibility Score with Respiratory Disease and Exacerbations

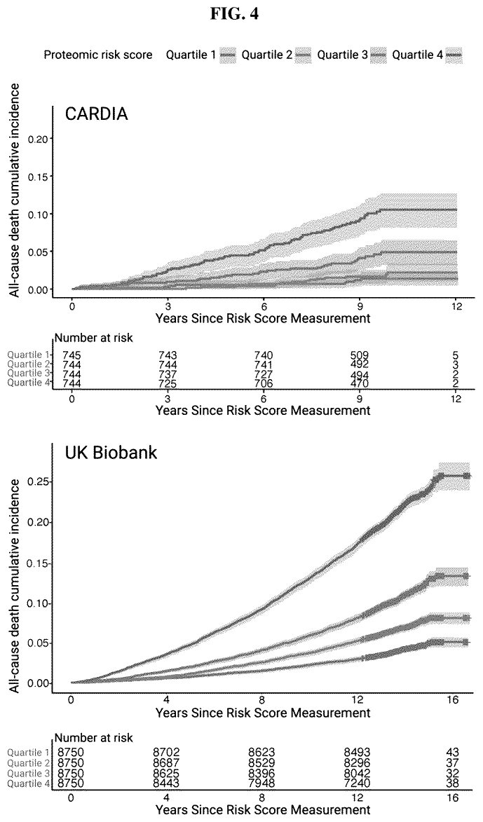

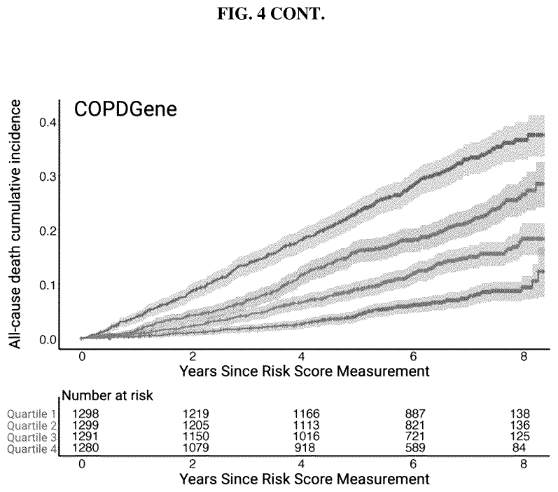

The unique set of large-scale population data in UKBB was used to understand the association between the susceptibility score and incident respiratory diseases. Each standard deviation higher susceptibility score was associated with increased risk of incident COPD (HR 1.84; 95% CI 1.71-1.98). Detailed data on respiratory exacerbations in the smoking-enriched population in COPDGene was used to examine associations between the susceptibility score and respiratory morbidity. In COPDGene, each standard deviation higher susceptibility score was associated with higher odds of one of more future respiratory exacerbations (OR 1.10, 95% CI 1.02-1.19) and future severe exacerbations (OR 1.17, 95% CI 1.08-1.27). Furthermore, the susceptibility score was associated with higher frequency of future exacerbations (IRR 1.28, 95% CI 1.19-1.38) and severe exacerbations (IRR 1.33, 95% CI 1.20-1.48). Associations by susceptibility score quartile are displayed in FIG. 4.

Susceptibility Score Among Those without Lung Disease at Baseline

In multivariable sensitivity analyses, the cohorts were restricted to those without lung disease at baseline and found similar results as with the entire cohort. There was a 52% higher risk of all-cause death in UKBB (HR 1.52; 95% CI 1.46-1.58) and 89% higher risk in COPDGene (HR 1.89; 95% CI 1.61-2.22) associated with one standard-deviation higher susceptibility score. In UKBB, this was also associated with 2.39 times the risk of respiratory death (HR, 2.34; 95% CI 2.07-2.66). Among those without baseline respiratory disease, there was only one respiratory death in COPDGene and therefore a hazard ratio was not able to be calculated. It was also found in COPDGene that one standard deviation higher susceptibility score was associated with higher odds of one of more future respiratory exacerbations (OR 1.13, 95% CI 1.00-1.28) but not risk of future severe exacerbations (OR 1.10, 95% CI 0.94-1.28). Associations by susceptibility score quartile are displayed in Table 11.

TABLE 11
Association between quartile of susceptibility score and respiratory
outcomes among those without baseline lung disease

| Outcome | Quartile 1 | Quartile 2 | Quartile 3 | Quartile 4 |
|---|---|---|---|---|
| All-cause | Ref | HR 1.06 | HR 2.14 | HR 3.89 |
| mortality | | (95% CI | (95% CI | (95% CI |
| | | 0.58-1.93) | 1.24-3.68) | 2.28-6.64) |
| Respiratory | Ref | OR 1.28 | OR 1.17 | HR 1.47 |
| execerbation | | (95% CI | (95% CI | (95% CI |
| | | 0.96-1.71) | 0.86-1.60) | 1.06-2.06) |
| Severe | Ref | OR 0.96 | OR 0.91 | OR 1.21 |
| execerbation | | (95% CI | (95% CI | (95% CI |
| | | 0.64-1.42) | 0.50-1.38) | 0.80-1.86) |

P-value <0.05 in bold. COPDGene analyses adjusted for age, sex, race, BMI, smoking status, pack years, baseline post-bronchodilator FEV1 percent predicted, WBC, platelets, study center, and income

### REFERENCES

The following references are incorporated by reference in their entireties.
1. Momtazmanesh S, Moghaddam S S, Ghamari S H, et al. Global burden of chronic respiratory diseases and risk factors, 1990-2019: an update from the Global Burden of Disease Study 2019. eClinicalMedicine. 2023;59.
2. Fletcher C, Peto R. The natural history of chronic airflow obstruction. Br Med J. 1977;1 (6077): 1645-1648.
3. Mannino D M, Davis K J. Lung function decline and outcomes in an elderly population. Thorax. 2006;61 (6): 472-477.
4. Mannino D M, Reichert M M, Davis K J. Lung Function Decline and Outcomes in an Adult Population. Am J Respir Crit Care Med. 2006;173 (9): 985-990.
5. Backman H, Blomberg A, Lundquist A, et al. Lung Function Trajectories and Associated Mortality Among Adults with and without Airway Obstruction. Am J Respir Crit Care Med. Published online Jul. 17, 2023.
6. Marott J L, Ingebrigtsen T S, çolak Y, Vestbo J, Lange P. Lung Function Trajectories Leading to Chronic Obstructive Pulmonary Disease as Predictors of Exacerbations and Mortality. Am J Respir Crit Care Med. 2020; 202 (2): 210-218.
7. Washko G R, Colangelo L A, Estépar RSJ, et al. Adult Life-Course Trajectories of Lung Function and the Development of Emphysema: The CARDIA Lung Study. Am J Med. 2020; 133 (2): 222-230.e11.
8. Reyfman P A, Washko G R, Dransfield M T, Spira A, Han M K, Kalhan R. Defining Impaired Respiratory Health. A Paradigm Shift for Pulmonary Medicine. Am J Respir Crit Care Med. 2018; 198 (4): 440-446.
9. Melén E, Faner R, Allinson J P, et al. Lung-function trajectories: relevance and implementation in clinical practice. The Lancet. 2024;Published online 12 Mar. 2024.
10. U S Preventive Services Task Force (USPSTF). Screening for Chronic Obstructive Pulmonary Disease: U S Preventive Services Task Force Recommendation Statement. JAMA. 2016; 315 (13): 1372-1377.
11. Liu G y. H, Perry A s., Washko G r., et al. Proteomic Risk Score of Accelerated Decline Lung Function Trajectory and Future Respiratory Morbidity and Mortality (abstract). Am J Respir Crit Care Med. 2024;209: A6910.
12. Serban K A, Pratte K A, Strange C, et al. Unique and shared systemic biomarkers for emphysema in Alpha-1 Antitrypsin deficiency and chronic obstructive pulmonary disease. EBioMedicine. 2022; 84:104262.
13. Katz D H, Robbins J M, Deng S, et al. Proteomic profiling platforms head to head: Leveraging genetics and clinical traits to compare aptamer- and antibody-based methods. Sci Adv. 2022;8 (33): eabm5164.
14. Stewart J I, Moyle S, Criner G J, et al. Automated telecommunication to obtain longitudinal follow-up in a multicenter cross-sectional COPD study. COPD. 2012; 9 (5): 466-472.
15. Gonzales T I, Westgate K, Strain T, et al. Cardiorespiratory fitness assessment using risk-stratified exercise testing and dose-response relationships with disease outcomes. Sci Rep. 2021; 11 (1): 15315.
16. Labaki W W, Gu T, Murray S, et al. Causes of and Clinical Features Associated with Death in Tobacco Cigarette Users by Lung Function Impairment. Am J Respir Crit Care Med. 2023;208 (4): 451-460.
17. Wells J M, Washko G R, Han M K, et al. Pulmonary arterial enlargement and acute exacerbations of COPD. N Engl J Med. 2012; 367 (10): 913-921.
18. Dennis G, Sherman B T, Hosack D A, et al. DAVID: Database for Annotation, Visualization, and Integrated Discovery. Genome Biol. 2003; 4 (5): P3.
19. Uhlén M, Fagerberg L, Hallström B M, et al. Tissue-based map of the human proteome. Science. 2015;347 (6220): 1260419.
20. Bhuva D D, Cursons J, Davis M J. Stable gene expression for normalisation and single-sample scoring. Nucleic Acids Res. 2020; 48 (19): e113.
21. Townsend P, Phillimore P, Beattie A. Health and Deprivation: Inequality and the North. Routledge; 1988.
22. Shrine N, Guyatt A L, Erzurumluoglu A M, et al. New genetic signals for lung function highlight pathways and chronic obstructive pulmonary disease associations across multiple ancestries. Nat Genet. 2019; 51 (3): 481-493.
23. Wain L V, Shrine N, Artigas M S, et al. Genome-wide association analyses for lung function and chronic obstructive pulmonary disease identify new loci and potential druggable targets. Nat Genet. 2017;49 (3): 416-425.
24. Moll M, Sakornsakolpat P, Shrine N, et al. Chronic obstructive pulmonary disease and related phenotypes: polygenic risk scores in population-based and case-control cohorts. Lancet Respir Med. 2020; 8 (7): 696-708.
25. Yang I A, Jenkins C R, Salvi S S. Chronic obstructive pulmonary disease in never-smokers: risk factors, pathogenesis, and implications for prevention and treatment. Lancet Respir Med. 2022; 10 (5): 497-511.
26. Ngo D, Pratte K A, Flexeder C, et al. Systemic Markers of Lung Function and FEV1 Decline across Diverse Cohorts. Ann Am Thorac Soc. Published online Jun. 23, 2023.
27. Higashimoto Y, Iwata T, Okada M, Satoh H, Fukuda K, Tohda Y. Serum biomarkers as predictors of lung function decline in chronic obstructive pulmonary disease. Respir Med. 2009; 103 (8): 1231-1238.
28. Zemans R L, Jacobson S, Keene J, et al. Multiple biomarkers predict disease severity, progression and mortality in COPD. Respir Res. 2017; 18:117.
29. Dahl M, Vestbo J, Lange P, Bojesen S E, Tybjærg-Hansen A, Nordestgaard B G. C-reactive Protein As a Predictor of Prognosis in Chronic Obstructive Pulmonary Disease. Am J Respir Crit Care Med. 2007; 175 (3): 250-255.
30. Celli B, Locantore N, Yates J C, et al. Markers of disease activity in COPD: an 8-year mortality study in the ECLIPSE cohort. Eur Respir J. 2021; 57 (3): 2001339.
31. Bui D S, Agusti A, Walters H, et al. Lung function trajectory and biomarkers in the Tasmanian Longitudinal Health Study. ERJ Open Res. 2021; 7 (3).
32. Attalah H L, Wu Y, Alaoui-E1-Azher M, et al. Induction of type-IIA secretory phospholipase A2 in animal models of acute lung injury. Eur Respir J. 2003; 21 (6): 1040-1045.
33. Yamashita J I, Ogawa M, Shirakusa T. Increased expression of membrane-associated phospholipase A2 in the lower respiratory tract of asymptomatic cigarette smokers. Respir Med. 1996; 90 (8): 479-483.
34. Pniewska E, Pawliczak R. The Involvement of Phospholipases A2 in Asthma and Chronic Obstructive Pulmonary Disease. Mediators Inflamm. 2013; 2013:793505.
35. Vallath S, Hynds R E, Succony L, Janes S M, Giangreco A. Targeting EGFR signalling in chronic lung disease: therapeutic challenges and opportunities. Eur Respir J. 2014; 44 (2): 513-522.
36. Lai H Y, Rogers D F. Mucus hypersecretion in asthma: intracellular signalling pathways as targets for pharmacotherapy. Curr Opin Allergy Clin Immunol. 2010; 10 (1): 67.
37. Burgel P R, Nadel J A. Epidermal growth factor receptor-mediated innate immune responses and their roles in airway diseases. Eur Respir J. 2008;32 (4): 1068-1081.
38. Noskovičová N, Heinzelmann K, Burgstaller G, Behr J, Eickelberg O. Cub domain-containing protein 1 negatively regulates TGF-β signaling and myofibroblast differentiation. Am J Physiol-Lung Cell Mol Physiol. 2018; 314 (5): L695-L707.
39. Kenyon N J, Ward R W, McGrew G, Last J A. TGF-1 causes airway fibrosis and increased collagen I and III mRNA in mice. Thorax. 2003; 58 (9): 772-777.
40. Kang L, Wang X, Wang J, Guo J, Zhang W, Lei R. NRF1 knockdown alleviates lipopolysaccharide-induced pulmonary inflammatory injury by upregulating DKK3 and inhibiting the GSK-3B/B-catenin pathway. Clin Exp Immunol. Published online Jul. 4, 2023: uxad071.
41. Schupp J C, Adams T S, Cosme C, et al. Integrated Single-Cell Atlas of Endothelial Cells of the Human Lung. Circulation. 2021; 144 (4): 286-302.
42. Chai L, Feng W, Zhai C, et al. The association between cystatin C and COPD: a meta-analysis and systematic review. BMC Pulm Med. 2020; 20 (1): 182.
43. Wang N, Yuan Y, Bai X, Han W, Han L, Qing B. Association of cathepsin B and cystatin C with an age-related pulmonary subclinical state in a healthy Chinese population. Ther Adv Respir Dis. 2020; 14:1753466620921751.
44. Hendrickson C M, Kwong Y D, Belzer A G, Shlipak M G, Matthay M A, Liu K D. Higher plasma cystatin C is associated with mortality after acute respiratory distress syndrome: findings from a Fluid and Catheter Treatment Trial (FACTT) substudy. Crit Care Lond Engl. 2020; 24 (1): 416.
45. Duan A, Huang Z, Zhao Z, et al. The potential of cystatin C as a predictive biomarker in pulmonary hypertension. BMC Pulm Med. 2023;23 (1): 311.
46. Mitsune A, Yamada M, Fujino N, et al. Upregulation of leukocyte immunoglobulin-like receptor B4 on interstitial macrophages in COPD; their possible protective role against emphysema formation. Respir Res. 2021; 22 (1): 232.
47. Lavis P, Bondue B, Cardozo A K. The Dual Role of Chemerin in Lung Diseases. Cells. 2024;13 (2): 171.
48. Carrión M, Frommer K W, Pérez-García S, Müller-Ladner U, Gomariz R P, Neumann E. The Adipokine Network in Rheumatic Joint Diseases. Int J Mol Sci. 2019; 20 (17): 4091.
49. Axelsson G T, Gudmundsson G, Pratte K A, et al. The Proteomic Profile of Interstitial Lung Abnormalities. Am J Respir Crit Care Med. 2022;206 (3): 337-346.
50. LeBleu V S, Teng Y, O'Connell J T, et al. Identification of human epididymis protein-4 as a fibroblast-derived mediator of fibrosis. Nat Med. 2013; 19 (2): 227-231.
51. Maddali M V, Moore A R, Sinha P, et al. Molecular Endotypes of Idiopathic Pulmonary Fibrosis: A Latent Class Analysis of Two Multicenter Observational Cohorts. Am J Respir Crit Care Med. Published online Jun. 24, 2024.
52. Raghu G, Richeldi L, Jagerschmidt A, et al. Idiopathic Pulmonary Fibrosis: Prospective, Case-Controlled Study of Natural History and Circulating Biomarkers. Chest. 2018; 154 (6): 1359-1370.
53. Cruz L C, Habibovic A, Dempsey B, et al. Identification of tyrosine brominated extracellular matrix proteins in normal and fibrotic lung tissues. Redox Biol. 2024; 71:103102.
54. Dessie E Y, Ding L, Mersha T B. Integrative analysis identifies gene signatures mediating the effect of DNA methylation on asthma severity and lung function. Clin Epigenetics. 2024; 16:15.
55. Nguyen H N, Jeong Y, Kim Y, et al. Leukemia inhibitory factor (LIF) receptor amplifies pathogenic activation of fibroblasts in lung fibrosis. BioRxiv Prepr Serv Biol. Published online May 23, 2024:2024.05.21.595153.
56. Knight D A, Lydell C P, Zhou D, Weir T D, Robert Schellenb erg R, Bai T R. Leukemia inhibitory factor (LIF) and LIF receptor in human lung. Distribution and regulation of LIF release. Am J Respir Cell Mol Biol. 1999;20 (4): 834-841.
57. Lamontagne M, Bérubé J C, Obeidat M, et al. Leveraging lung tissue transcriptome to uncover candidate causal genes in COPD genetic associations. Hum Mol Genet. 2018; 27 (10): 1819-1829.
58. Wortley P M, Caraballo R S, Pederson L L, Pechacek T F. Exposure to secondhand smoke in the workplace: serum cotinine by occupation. J Occup Environ Med. 2002;44 (6): 503-509.
59. Niu Y, Lin A, Luo P, et al. Prognosis of Lung Adenocarcinoma Patients With NTRK3 Mutations to Immune Checkpoint Inhibitors. Front Pharmacol. 2020; 11:1213.
60. plvarez-Díaz S, Valle N, García J M, et al. Cystatin D is a candidate tumor suppressor gene induced by vitamin D in human colon cancer cells. J Clin Invest. 2009; 119 (8): 2343-2358.
61. Mujalli A, Alghamdi K S, Nasser K K, et al. Bioinformatics insights into the genes and pathways on severe COVID-19 pathology in patients with comorbidities. Front Physiol. 2022; 13:1045469.
62. Wain L V, Shrine N, Miller S, et al. Novel insights into the genetics of smoking behaviour, lung function, and chronic obstructive pulmonary disease (U K BiLEVE): a genetic association study in U K Biobank. Lancet Respir Med. 2015;3 (10): 769-781.
63. Westphal N J, Seasholtz A F. CRH-B P: the regulation and function of a phylogenetically conserved binding protein. Front Biosci-Landmark. 2006;11 (2): 1878-1891.
64. Byers H M, Dagle J M, Klein J M, et al. Variations in CRHR1 are associated with persistent pulmonary hypertension of the newborn. Pediatr Res. 2012; 71 (2): 162-167.
65. Monestier O, Blanquet V. WFIKKN1 and WFIKKN2: “Companion” proteins regulating TGFB activity. Cytokine Growth Factor Rev. 2016; 32:75-84.
66. Bonnefond A, Yengo L, Dechaume A, et al. Relationship between salivary/pancreatic amylase and body mass index: a systems biology approach. BMC Med. 2017;15 (1): 37.
67. Muller O, Pradervand S, Berger S, et al. Identification of corticosteroid-regulated genes in cardiomyocytes by serial analysis of gene expression. Genomics. 2007; 89 (3): 370-377.
68. Tang F, Pacheco MTF, Chen P, Liang D, Li W. Secretogranin III promotes angiogenesis through MEK/ERK signaling pathway. Biochem Biophys Res Commun. 2018; 495 (1): 781-786.
69. Gorlov I P, Meyer P, Liloglou T, et al. Seizure 6-like (SEZ6L) gene and risk for lung cancer. Cancer Res. 2007; 67 (17): 8406-8411.
70. Carayol J, Chabert C, Di Cara A, et al. Protein quantitative trait locus study in obesity during weight-loss identifies a leptin regulator. Nat Commun. 2017;8 (1): 2084.
71. Chomette L, Hupkens E, Romitti M, et al. Pediatric pulmonary arterial hypertension due to a novel homozygous GDF2 missense variant affecting BMP9 processing and activity. Am J Med Genet A. 2023;191 (8): 2064-2073.
72. Rong B, Liu Y, Li M, Fu T, Gao W, Liu H. Correlation of serum levels of HIF-la and IL-19 with the disease progression of COPD: a retrospective study. Int J Chron Obstruct Pulmon Dis. 2018; 13:3791-3803.
73. Liao S C, Cheng Y C, Wang Y C, et al. IL-19 induced Th2 cytokines and was up-regulated in asthma patients. J Immunol Baltim Md 1950. 2004; 173 (11): 6712-6718.
74. Wang Y, Sun S, Wang K, et al. Interleukin-19 Aggravates Pulmonary Fibrosis via Activating Fibroblast through TGF-B/Smad Pathway. Mediators Inflamm. 2022; 2022:6755407.

What is claimed is:

1. A method comprising quantitating, within a biological sample from a subject, the levels of a panel of biomarkers comprising two or more biomarkers selected from: SLAM family member 7; Interleukin-36 alpha; Oxytocin-neurophysin 1; Fatty acid-binding protein, adipocyte; Axin-2; Interleukin-17 receptor E; Orphan sodium- and chloride-dependent neurotransmitter transporter NTT5; BPI fold-containing family B member 1; Galectin-3-binding protein; Nuclear distribution protein nudE-like 1; C-C motif chemokine 22; Liver-expressed antimicrobial peptide 2; Tissue-type plasminogen activator; Protein S100-A9; Marginal zone B- and B1-cell-specific protein; C-C motif chemokine 18; Piezo-type mechanosensitive ion channel component 1; Glutathione S-transferase A1; Ribonuclease K6; Amiloride-sensitive amine oxidase [copper-containing]; Gastrokine-2; Protocadherin gamma-C3; Macrophage scavenger receptor types I and II: Extracellular domain; Lipopolysaccharide-binding protein; C-C motif chemokine 3; Peroxidasin homolog; Scavenger receptor class B member 1; Ephrin type-B receptor 3; SLAM family member 8; Endothelial cell-derived lipase; Thrombospondin-2; Glycerol-3-phosphate dehydrogenase [NAD (+)], cytoplasmic; Estradiol 17-beta-dehydrogenase 1; Trafficking protein particle complex subunit 3; Serine protease HTRA1; Complement C3d fragment; Macrophage-capping protein; DnaJ homolog subfamily B member 9; Triggering receptor expressed on myeloid cells 2; Angiopoietin-2; Complement C3b, inactivated; Retinoic acid receptor responder protein 2; Complement component C9; Malignant T-cell-amplified sequence 1; Leukocyte immunoglobulin-like receptor subfamily A member 5; Glycerol-3-phosphate dehydrogenase [NAD (+)], cytoplasmic; Histone H2A type 1-A; Choline/ethanolamine kinase; Histone H2B type 1-K; Insulin; Myeloblastin; Growth hormone receptor; Serine/arginine-rich splicing factor 7; Interleukin-1 receptor antagonist protein; T-cell immunoglobulin and mucin domain-containing protein 4; Vesicular integral-membrane protein VIP36; V-set and transmembrane domain-containing protein 2-like protein; Transformer-2 protein homolog beta; E-selectin; Cytosolic Fe-S cluster assembly factor NUBP2; Ficolin-1; Gamma-glutamyl hydrolase; Leukocyte cell-derived chemotaxin-2; Inhibin beta A chain; Collagen alpha-3 (VI) chain: Bovine pancreatic trypsin inhibitor/Kunitz inhibitor domain, isoform 1; Marginal zone B- and B1-cell-specific protein; Regulator of G-protein signaling 4; C-C motif chemokine 21; Angiopoietin-2; Retinoblastoma-like protein 2; 5-hydroxytryptamine receptor 6; Protein S100-A12; Inhibin beta C chain; Bone sialoprotein 2; V-set and immunoglobulin domain-containing protein 4; Intercellular adhesion molecule 1; Triggering receptor expressed on myeloid cells 1; Collagen alpha-1 (XXVIII) chain; Olfactomedin-like protein 3; Tumor necrosis factor receptor superfamily member 10A; Serine/threonine-protein phosphatase 1 regulatory subunit 10; IGF-like family receptor 1; Thyroid transcription factor 1-associated protein 26; Tyrosine-protein phosphatase non-receptor type 7; G antigen 2; Replication initiator 1; ADAMTS-like protein 2; WAP four-disulfide core domain protein 2; Protein phosphatase 1 regulatory subunit 1A; PIH1 domain-containing protein 2; Rho GTPase-activating protein 36; 2-methoxy-6-polyprenyl-1,4-benzoquinol methylase, mitochondrial; Colipase-like protein 1; Sialic acid-binding Ig-like lectin 12: Ig-like C2-type 2 domain, Isoform short; Osteopetrosis-associated transmembrane protein 1; Cystatin B; Delta-like protein 1; Tumor necrosis factor receptor superfamily member 6; Insulin-like growth factor-binding protein 4; Lysozyme C; Protein FAM19A5; FXYD domain-containing ion transport regulator 6; Alpha-1-antichymotrypsin complex; Trafficking protein particle complex subunit 5; Protein FAM234B: N-term; Heat shock 70 kDa protein 1B; Cellular retinoic acid-binding protein 2; Z-DNA-binding protein 1; Transcriptional repressor protein YY1; Beta-2-microglobulin; Triggering receptor expressed on myeloid cells 2; Far upstream element-binding protein 2; Fc receptor-like protein 3; ATPase family AAA domain-containing protein 2; Galectin-9; Calcipressin-3; Plastin-2; Leucine-rich repeats and immunoglobulin-like domains protein 1; Complement factor B; Heat shock 70 kDa protein 1A; E3 ubiquitin-protein ligase RAD18; Syndecan-3; Antizyme inhibitor 1; Complement component C9; Ubiquitin-conjugating enzyme E2 G2; Interleukin-2; Holo-Transcobalamin-2; Ephrin-A1; Oncoprotein-induced transcript 3 protein; Stathmin-3; Tumor necrosis factor receptor superfamily member 1B; Asialoglycoprotein receptor 1; Methionine-R-sulfoxide reductase B1; CREB-binding protein; Serine/arginine-rich splicing factor 6; Synembryn-A; Myc target protein 1; Heparin cofactor 2; Ataxin-2-binding protein 1; GTPase IMAP family member 6; Galectin-4; Leukocyte immunoglobulin-like receptor subfamily A member 5; Thymidine kinase, cytosolic; ADP-ribosylation factor-like protein 15; DCN1-like protein 1; Coagulation factor IXab; Coagulation factor IX; Heat shock 70 kDa protein 1A; Tumor necrosis factor receptor superfamily member 1B; Sperm surface protein Sp17; Complement Clr subcomponent-like protein; Peptidyl-prolyl cis-trans isomerase C; Protein FAM204A; Interleukin-17C; Gamma-aminobutyric acid receptor-associated protein-like 1; Legumain; Inactive ribonuclease-like protein 10; Vitronectin; HLA class II histocompatibility antigen, DR beta 3 chain; Heat shock 70 kDa protein 1A; Tolloid-like protein 1; Macrophage receptor MARCO; Four and a half LIM domains protein 1; Gamma-aminobutyric acid receptor-associated protein; Killer cell immunoglobulin-like receptor 2DS4; Protein BUD31 homolog; Alpha-aminoadipic semialdehyde dehydrogenase; Inactive serine protease 35; Ubiquitin-conjugating enzyme E2 D2; Complement factor D; Dipeptidyl peptidase 1; Heat shock cognate 71 kDa protein; Antileukoproteinase; WD repeat-containing protein 5; Out at first protein homolog; Neuroblastoma suppressor of tumorigenicity 1; Tumor necrosis factor ligand superfamily member 11; Kynureninase; Ephrin-A4; EGF-containing fibulin-like extracellular matrix protein 1; Tumor necrosis factor receptor superfamily member 19L; Serum amyloid P-component; Complement C2; Follistatin-related protein 3; Cystatin-C; Serine/threonine-protein kinase 17B; Protein RIC-3; Leukocyte immunoglobulin-like receptor subfamily B member 4; Metalloproteinase inhibitor 1; C-X-C motif chemokine 16; Fibulin-5; Transmembrane gamma-carboxyglutamic acid protein 1: Cytoplasmic domain; C4b-binding protein alpha chain; Tumor necrosis factor ligand superfamily member 15; Secreted and transmembrane protein 1; Heat shock 70 kDa protein 1A; Complement Clq tumor necrosis factor-related protein 5; Ganglioside GM2 activator; Gamma-aminobutyric acid receptor-associated protein; DnaJ homolog subfamily C member 11; Atrial natriuretic factor; D-dimer; RecQ-mediated genome instability protein 1; von Willebrand factor A domain-containing protein 1; Pituitary adenylate cyclase-activating polypeptide 38; Uncharacterized protein C14orf93; Gem-associated protein 6; Matrix Gla protein; Guanine nucleotide exchange factor VAV3; Inactive dipeptidyl peptidase 10; Plastin-2; Fibrinogen gamma chain; Paraspeckle component 1; Calcium-dependent phospholipase A2; Complement Clq tumor necrosis factor-related protein 1; Integrin a5b1; Calpain-9; Cadherin-1; Complement factor H-related protein 1; Complement factor H; Antithrombin-III; Apolipoprotein C-I; Neurogenic locus notch homolog protein 1; Heat shock 70 kDa protein 12A; Kallistatin; Glutathione peroxidase 3; Kallistatin; Prostasin; Repulsive guidance molecule A; Serum albumin; Epidermal growth factor receptor; Protein SCO1 homolog, mitochondrial; Mitogen-activated protein kinase 6; B melanoma antigen 3; Heparan-sulfate 6-O-sulfotransferase 3; Platelet endothelial aggregation receptor 1: Extracellular domain; Neural cell adhesion molecule L1-like protein; Neuropeptide S; Ephrin type-A receptor 4; Beta-hexosaminidase subunit beta; RGM domain family member B; Leucine-rich repeats and immunoglobulin-like domains protein 3; Melanoma-associated antigen MUC18; Zinc finger protein 230; Inter-alpha-trypsin inhibitor heavy chain H2; Tetratricopeptide repeat protein 33; Leukemia inhibitory factor receptor; Glycosaminoglycan xylosylkinase; Methylglutaconyl-CoA hydratase, mitochondrial; Endothelial cell-selective adhesion molecule; Neurotrimin; MAD2L1-binding protein; Growth/differentiation factor 2; Glucosidase 2 subunit beta; Desmoglein-2; Contactin-1; Neural cell adhesion molecule L1-like protein; Inter-alpha-trypsin inhibitor heavy chain H1; Slit homolog 2 protein; Heat shock 70 kDa protein 1A; Sodium/potassium-transporting ATPase subunit beta-2; Speriolin-like protein; Leucine-rich repeat transmembrane neuronal protein 2; Endothelial cell-selective adhesion molecule; Mediator of RNA polymerase II transcription subunit 11; Anosmin-1; Microtubule-associated proteins 1A/1B light chain 3 beta 2; Apolipoprotein A-IV; Beta-1,4-galactosyltransferase 2; Neurotrimin; Interleukin-1 receptor type 1; Apolipoprotein D; Dihydrolipoyl dehydrogenase, mitochondrial; Gliomedin; Serine protease 57; Interleukin-19; Inactive tyrosine-protein kinase transmembrane receptor ROR1; Carbonyl reductase [NADPH] 3; Kunitz-type protease inhibitor 2; Endothelial cell-specific molecule 1; Bone morphogenetic protein receptor type-1A; 15 kDa selenoprotein; Complement Clq-like protein 3; von Willebrand factor A domain-containing protein 2; Anthrax toxin receptor 2; Semaphorin-3G; Tyrosine-protein kinase SYK: Protein kinase domain; Normal mucosa of esophagus-specific gene 1 protein; Ciliary neurotrophic factor receptor subunit alpha; SLIT and NTRK-like protein 4; Protocadherin-9; Uronyl 2-sulfotransferase; N-acetylglucosamine-1-phosphodiester alpha-N-acetylglucosaminidase; Tubulointerstitial nephritis antigen-like; Cystatin-M; Dickkopf-related protein 3; Neural cell adhesion molecule 2; Polypeptide N-acetylgalactosaminyltransferase 4; Ephrin type-A receptor 4; Vesicular, overexpressed in cancer, prosurvival protein 1; Dual specificity protein phosphatase 13 isoform A; Ectonucleotide pyrophosphatase/phosphodiesterase family member 5; NT-3 growth factor receptor; Endothelial cell-selective adhesion molecule; Dipeptidase 1; 3-mercaptopyruvate sulfurtransferase; Trafficking protein particle complex subunit 6B; Cell adhesion molecule 2; Anthrax toxin receptor 1; Dickkopf-related protein 4; Gamma-glutamyltransferase 5; Seizure 6-like protein; EMILIN-3: region 1; Klotho; Protocadherin-10: Extracellular domain; Transmembrane protein 132A; Syntaxin-1A; WAP, Kazal, immunoglobulin, Kunitz and NTR domain-containing protein 2; Interleukin-2 receptor subunit beta; Acetylcholinesterase; Contactin-2; Coiled-coil domain-containing protein 126; Alpha-amylase 2B; Integrin alpha V beta 3; WAP, Kazal, immunoglobulin, Kunitz and NTR domain-containing protein 2; Interleukin-1 Receptor accessory protein; Glyoxylate reductase/hydroxypyruvate reductase; Neural cell adhesion molecule 1, 120 kDa isoform; Secretogranin-3; Neural cell adhesion molecule 1; Netrin receptor UNC5D; Alpha-amylase 2B; Alpha-1,3-mannosyltransferase ALG2; Matrix-remodeling-associated protein 8: Extracellular domain; Receptor-type tyrosine-protein phosphatase delta; Interleukin-1 Receptor accessory protein; A disintegrin and metalloproteinase with thrombospondin motifs 13; Dickkopf-related protein 3; Gamma-enolase; Glypican-3; Kynurenine-oxoglutarate transaminase 3; Calcium and integrin-binding protein 1; Prostaglandin F2 receptor negative regulator; SLIT and NTRK-like protein 5; Activating signal cointegrator 1 complex subunit 2; DNA-directed RNA polymerases I, II, and III subunit RPABC4; Brevican core protein; Peroxisomal carnitine O-octanoyltransferase; Neuronal pentraxin receptor; Ciliary neurotrophic factor receptor subunit alpha; Netrin receptor UNC5D; Biglycan; Adhesion G protein-coupled receptor F5; SLIT and NTRK-like protein 1; Serine-rich single-pass membrane protein 1; IgLON family member 5; Voltage-dependent calcium channel subunit alpha-2/delta-3; Pancreatic alpha-amylase; Ephrin type-A receptor 6; Interleukin-35; Integrin αIIb1; Adiponectin; Fc receptor-like protein 4: Extracellular domain; Histone-lysine N-methyltransferase EHMT2; THAP domain-containing protein 4; Interleukin-27 subunit beta; Myelin-associated glycoprotein; Pancreatic triacylglycerol lipase; Cerebellin-2; Cysteine-rich with EGF-like domain protein 1; Ecto-ADP-ribosyltransferase 3; Ecto-ADP-ribosyltransferase 3; AP-1 complex-associated regulatory protein; Neuronal pentraxin receptor; Neurocan core protein; Cerebellin-1; Cystatin-SA; ADP-ribosylation factor 4; DCC; Amyloid-like protein 1; Pyruvate dehydrogenase E1 component subunit alpha, testis-specific form, mitochondrial; Advanced glycosylation end product-specific receptor, soluble; Galactosylgalactosylxylosylprotein 3-beta-glucuronosyltransferase 1; Oligodendrocyte-myelin glycoprotein; Lactase-phlorizin hydrolase; Insulin-like growth factor-binding protein 1; SLIT and NTRK-like protein 3; Carbonic anhydrase 6; and Carbonic anhydrase 6.

2. The method of claim 1, within a biological sample from a subject, the levels of a panel of biomarkers comprising two or more biomarkers selected from: Nuclear distribution protein nudE-like 1; Ribonuclease pancreatic; Estradiol 17-beta-dehydrogenase 1; Phospholipase A2, membrane associated; Protein S100-A9; Triggering receptor expressed on myeloid cells 1; Ribonuclease K6; Cystatin B; Osteopetrosis-associated transmembrane protein 1; Delta-like protein 1; Retinoic acid receptor responder protein 2; 5-hydroxytryptamine receptor 6; 2-methoxy-6-polyprenyl-1,4-benzoquinol methylase, mitochondrial; Collagen alpha-1 (XXVIII) chain; Collagen alpha-3 (VI) chain: Bovine pancreatic trypsin inhibitor/Kunitz inhibitor domain, isoform 1; Corticotropin-releasing factor-binding protein; Complement Clr subcomponent-like protein; Gamma-aminobutyric acid receptor-associated protein; Gamma-aminobutyric acid receptor-associated protein-like 1; Serine/threonine-protein kinase 17B; Leukemia inhibitory factor receptor; Hedgehog-interacting protein; Semaphorin-3G; Protein FAM177A1; Neural cell adhesion molecule 1; SLIT and NTRK-like protein 4; Tubulointerstitial nephritis antigen-like; NT-3 growth factor receptor; Uronyl 2-sulfotransferase; Protocadherin-9; Seizure 6-like protein; Neurogenic locus notch homolog protein 2; WAP, Kazal, immunoglobulin, Kunitz and NTR domain-containing protein 2; Dickkopf-related protein 3; IgLON family member 5; Histatin-3; WAP, Kazal, immunoglobulin, Kunitz and NTR domain-containing protein 2; Brevican core protein; Cerebellin-2; Myelin-associated glycoprotein; Alpha-amylase 2B; Neurocan core protein; Mannan-binding lectin serine protease 1: Mannan-binding lectin serine protease 1 heavy chain; Oligodendrocyte-myelin glycoprotein; Cystatin-D; Amyloid-like protein 1; Carbonic anhydrase 6; and Carbonic anhydrase 6.

3. The method of claim 1, wherein the two or more biomarkers are a protein or a cDNA.

4-8. (canceled)

9. The method of claim 1, wherein the biological sample is plasma or blood.

10. The method of claim 1, wherein the biological sample is obtained from a subject that suffers from impaired respiratory health.

11. The method of claim 1, wherein the biological sample is obtained from a subject that is at risk of impaired respiratory health.

12. The method of claim 1, wherein the biomarkers are associated with advanced parenchymal diseases selected from lung injury, inflammation, pulmonary fibrosis, chronic obstructive pulmonary disease (COPD) and emphysema, and lung cancer.

13. (canceled)

14. A method of assessing the lung health of a subject comprising:
(a) quantifying the levels of a panel of biomarkers according to the method of claim 1; and
(b) comparing the levels to a control or threshold value for each, wherein a significant difference between the level of one or more of the biomarkers from the control or threshold value is indicative of a impaired respiratory health.

15-17. (canceled)

18. The method of claim 14, wherein comparing the level of each biomarker in the panel to a control or threshold value for each comprises calculating the difference between the level of each biomarker in the panel to a control or threshold value for each.

19. The method of claim 14, wherein comparing the level of each biomarkers in the panel to a control or threshold value for each further comprises dividing the difference by the standard deviation for the population of healthy subjects to generate a biomarkers score for each biomarker.

20. The method of claim 14, further comprising generating a composite score for the panel of biomarkers by comparing the level of each biomarker in the panel to a control or threshold value for each to generate a biomarkers score for each biomarker and combining the biomarkers scores to generate the composite score; and comparing the composite score to a composite threshold value, wherein a significant difference between the composite score to a composite threshold value is indicative of a impaired respiratory health.

21. The method of claim 14, wherein the control or threshold value for each biomarkers is based on a population average for healthy subjects.

22. The method of claim 21, wherein the biomarkers score for each biomarker in the panel is calculated by calculating the difference between the level of each biomarkers biomarker in the panel to a control or threshold value for each and dividing the difference by the standard deviation for the population of healthy subjects.

23. (canceled)

24. A method of monitoring the lung health of a subject over time, comprising:
(a) assessing the lung health of the subject at a first timepoint by quantifying the levels of a panel of biomarkers in a first biological sample from the subject;
(b) assessing the lung health of the subject at a second timepoint by quantifying the levels of the panel of biomarkers in a second biological sample from the subject;
(c) comparing the levels at the first timepoint to the second timepoint;
wherein a significant difference between the level of one or more of the biomarkers from the first timepoint to the second timepoint is indicative of impaired respiratory health.

25-30. (canceled)

31. A system comprising a panel of biomarkers selected from Table 4 and Table 5.

32-33. (canceled)

34. The system of claim 31, wherein the panel of biomarkers selected from Table 4 and Table 5 comprise 20 or more biomarkers,

35-37. (canceled)

38. The system of claim 31, wherein the panel of biomarkers selected from Table 4 and Table 5 comprise 100 or fewer biomarkers.

39-44. (canceled)

45. The system of claim 31, wherein the biomarkers are proteins.

46. (canceled)

47. The system of claim 31, wherein the biomarkers are cDNAs.

48-51. (canceled)

52. The system of claim 31, wherein the biomarkers are RNAs.

53. A method of treating the lung health of a subject comprising assessing the levels of one or more biomarkers selected from Table 4 and Table 5 in a biological sample from the subject and, if the levels are indicative of impaired respiratory health, then administering a quantitative and/or responsive physiologic measure of airflow obstruction in a subject, administering a schedule of pulmonary rehabilitation, and/or administering systemic steroids and/or antibiotics.

54-56. (canceled)
